SEQUENCE LISTING (93 sequences)

SEQ ID NO: 1 - protein, 217 aa, synthetic construct
  REGION 1..217: note = Synthetic polymer.
APEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

SEQ ID NO: 2 - protein, 217 aa, synthetic construct
  REGION 1..217: note = Synthetic polymer.
APEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGK

SEQ ID NO: 3 - protein, 217 aa, synthetic construct
  REGION 1..217: note = Synthetic polymer.
APEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 4 - protein, 232 aa, synthetic construct
  REGION 1..232: note = Synthetic polymer.
EPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 232 aa, synthetic construct
  REGION 1..232: note = Synthetic polymer.
EPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 11 aa, synthetic construct
  REGION 1..11: note = Synthetic polymer.
SKYGPPCPSCP

SEQ ID NO: 7 - protein, 11 aa, synthetic construct
  REGION 1..11: note = Synthetic polymer.
SKYGPPCPPCP

SEQ ID NO: 8 - protein, 6 aa, synthetic construct
  REGION 1..6: note = Synthetic polymer.
SKYGPP

SEQ ID NO: 9 - protein, 6 aa, synthetic construct
  REGION 1..6: note = Synthetic polymer.
IEGRMD

SEQ ID NO: 10 - protein, 9 aa, synthetic construct
  REGION 1..9: note = Synthetic polymer.
GGGVPRDCG

SEQ ID NO: 11 - protein, 15 aa, synthetic construct
  REGION 1..15: note = Synthetic polymer.
IEGRMDGGGGAGGGG

SEQ ID NO: 12 - protein, 8 aa, synthetic construct
  REGION 1..8: note = Synthetic polymer.
GGGSGGGS

SEQ ID NO: 13 - protein, 12 aa, synthetic construct
  REGION 1..12: note = Synthetic polymer.
GGGSGGGGSGGG

SEQ ID NO: 14 - protein, 14 aa, synthetic construct
  REGION 1..14: note = Synthetic polymer.
EGKSSGSGSESKST

SEQ ID NO: 15 - protein, 4 aa, synthetic construct
  REGION 1..4: note = Synthetic polymer.
GGSG

SEQ ID NO: 16 - protein, 12 aa, synthetic construct
  REGION 1..12: note = Synthetic polymer.
GGSGGGSGGGSG

SEQ ID NO: 17 - protein, 15 aa, synthetic construct
  REGION 1..15: note = Synthetic polymer.
EAAAKEAAAKEAAAK

SEQ ID NO: 18 - protein, 20 aa, synthetic construct
  REGION 1..20: note = Synthetic polymer.
EAAAREAAAREAAAREAAAR

SEQ ID NO: 19 - protein, 17 aa, synthetic construct
  REGION 1..17: note = Synthetic polymer.
GGGGSGGGGSGGGGSAS

SEQ ID NO: 20 - protein, 9 aa, synthetic construct
  REGION 1..9: note = Synthetic polymer.
GGGGAGGGG

SEQ ID NO: 21 - skipped

SEQ ID NO: 22 - protein, 6 aa, synthetic construct
  REGION 1..6: note = Synthetic polymer.
GSGSGS

SEQ ID NO: 23 - protein, 10 aa, synthetic construct
  REGION 1..10: note = Synthetic polymer.
GSGSGSGSGS

SEQ ID NO: 24 - protein, 7 aa, synthetic construct
  REGION 1..7: note = Synthetic polymer.
GGGGSAS

SEQ ID NO: 25 - protein, 20 aa, synthetic construct
  REGION 1..20: note = Synthetic polymer.
APAPAPAPAPAPAPAPAPAP

SEQ ID NO: 26 - protein, 4 aa, synthetic construct
  REGION 1..4: note = Synthetic polymer.
CPPC

SEQ ID NO: 27 - protein, 5 aa, synthetic construct
  REGION 1..5: note = Synthetic polymer.
GGGGS

SEQ ID NO: 28 - protein, 10 aa, synthetic construct
  REGION 1..10: note = Synthetic polymer.
GGGGSGGGGS

SEQ ID NO: 29 - protein, 15 aa, synthetic construct
  REGION 1..15: note = Synthetic polymer.
GGGGSGGGGSGGGGS

SEQ ID NO: 30 - protein, 20 aa, synthetic construct
  REGION 1..20: note = Synthetic polymer.
GGGGSGGGGSGGGGSGGGGS

SEQ ID NO: 31 - protein, 25 aa, synthetic construct
  REGION 1..25: note = Synthetic polymer.
GGGGSGGGGSGGGGSGGGGSGGGGS

SEQ ID NO: 32 - protein, 30 aa, synthetic construct
  REGION 1..30: note = Synthetic polymer.
GGGGSGGGGSGGGGSGGGGSGGGGSGGGGS

SEQ ID NO: 33 - protein, 35 aa, synthetic construct
  REGION 1..35: note = Synthetic polymer.
GGGGSGGGGSGGGGSGGGGSGGGGSGGGGSGGGGS

SEQ ID NO: 34 - protein, 40 aa, synthetic construct
  REGION 1..40: note = Synthetic polymer.
GGGGSGGGGSGGGGSGGGGSGGGGSGGGGSGGGGSGGGGS

SEQ ID NO: 35 - protein, 16 aa, synthetic construct
  REGION 1..16: note = Synthetic polymer.
GGSGGSGGGGSGGGGS

SEQ ID NO: 36 - protein, 8 aa, synthetic construct
  REGION 1..8: note = Synthetic polymer.
GGGGGGGG

SEQ ID NO: 37 - protein, 6 aa, synthetic construct
  REGION 1..6: note = Synthetic polymer.
GGGGGG

SEQ ID NO: 38 - protein, 5 aa, synthetic construct
  REGION 1..5: note = Synthetic polymer.
EAAAK

SEQ ID NO: 39 - protein, 10 aa, synthetic construct
  REGION 1..10: note = Synthetic polymer.
EAAAKEAAAK

SEQ ID NO: 40 - protein, 15 aa, synthetic construct
  REGION 1..15: note = Synthetic polymer.
EAAAKEAAAKEAAAK

SEQ ID NO: 41 - protein, 12 aa, synthetic construct
  REGION 1..12: note = Synthetic polymer.
AEAAAKEAAAKA

SEQ ID NO: 42 - protein, 17 aa, synthetic construct
  REGION 1..17: note = Synthetic polymer.
AEAAAKEAAAKEAAAKA

SEQ ID NO: 43 - protein, 22 aa, synthetic construct
  REGION 1..22: note = Synthetic polymer.
AEAAAKEAAAKEAAAKEAAAKA

SEQ ID NO: 44 - protein, 27 aa, synthetic construct
  REGION 1..27: note = Synthetic polymer.
AEAAAKEAAAKEAAAKEAAAKEAAAKA

SEQ ID NO: 45 - protein, 46 aa, synthetic construct
  REGION 1..46: note = Synthetic polymer.
AEAAAKEAAAKEAAAKEAAAKALEAEAAAKEAAAKEAAAKEAAAKA

SEQ ID NO: 46 - protein, 5 aa, synthetic construct
  REGION 1..5: note = Synthetic polymer.
PAPAP

SEQ ID NO: 47 - protein, 18 aa, synthetic construct
  REGION 1..18: note = Synthetic polymer.
KESGSVSSEQLAQFRSLD

SEQ ID NO: 48 - protein, 12 aa, synthetic construct
  REGION 1..12: note = Synthetic polymer.
GSAGSAAGSGEF

SEQ ID NO: 49 - protein, 5 aa, synthetic construct
  REGION 1..5: note = Synthetic polymer.
GGGSE

SEQ ID NO: 50 - protein, 5 aa, synthetic construct
  REGION 1..5: note = Synthetic polymer.
GSESG

SEQ ID NO: 51 - protein, 5 aa, synthetic construct
  REGION 1..5: note = Synthetic polymer.
GSEGS

SEQ ID NO: 52 - protein, 35 aa, synthetic construct
  REGION 1..35: note = Synthetic polymer.
GEGGSGEGSSGEGSSSEGGGSEGGGSEGGGSEGGS

SEQ ID NO: 53 - protein, 234 aa, synthetic construct
  REGION 1..234: note = Synthetic polymer.
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 54 - protein, 234 aa, synthetic construct
  REGION 1..234: note = Synthetic polymer.
SKYGPPCPSCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 55 - protein, 234 aa, synthetic construct
  REGION 1..234: note = Synthetic polymer.
SKYGPPCPSCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 56 - protein, 234 aa, synthetic construct
  REGION 1..234: note = Synthetic polymer.
SKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 57 - protein, 234 aa, synthetic construct
  REGION 1..234: note = Synthetic polymer.
SKYGPPCPPCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 58 - protein, 234 aa, synthetic construct
  REGION 1..234: note = Synthetic polymer.
SKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 59 - protein, 120 aa, synthetic construct
  REGION 1..120: note = Synthetic polymer.
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 60 - protein, 126 aa, synthetic construct
  REGION 1..126: note = Synthetic polymer.
ETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 61 - protein, 182 aa, synthetic construct
  REGION 1..182: note = Synthetic polymer.
LQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 62 - protein, 235 aa, synthetic construct
  REGION 1..235: note = Synthetic polymer.
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 63 - protein, 182 aa, synthetic construct
  REGION 1..182: note = Synthetic polymer.
TMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 64 - protein, 131 aa, synthetic construct
  REGION 1..131: note = Synthetic polymer.
SSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 65 - protein, 346 aa, synthetic construct
  REGION 1..346: note = Synthetic polymer.
QVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 66 - protein, 125 aa, synthetic construct
  REGION 1..125: note = Synthetic polymer.
HPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICK

SEQ ID NO: 67 - protein, 168 aa, synthetic construct
  REGION 1..168: note = Synthetic polymer.
TYHFTYGETGKRLSELHSYHSSLTCFSEGTKVPAWGCCPASWKSFGSSCYFISSEEKVWSKSEQNCVEMGAHLVVFNTEAEQNFIVQQLNESFSYFLGLSDPQGNNNWQWIDKTPYEKNVRFWHLGEPNHSAEQCASIVFWKPTGWGWNDVICETRRNSICEMNKIYL

SEQ ID NO: 68 - protein, 118 aa, synthetic construct
  REGION 1..118: note = Synthetic polymer.
FGSSCYFISSEEKVWSKSEQNCVEMGAHLVVFNTEAEQNFIVQQLNESFSYFLGLSDPQGNNNWQWIDKTPYEKNVRFWHLGEPNHSAEQCASIVFWKPTGWGWNDVICETRRNSICE

SEQ ID NO: 69 - protein, 264 aa, synthetic construct
  REGION 1..264: note = Synthetic polymer.
PRFMGTISDVKTNVQLLKGRVDNISTLDSEIKKNSDGMEAAGVQIQMVNESLGYVRSQFLKLKTSVEKANAQIQILTRSWEEVSTLNAQIPELKSDLEKASALNTKIRALQGSLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEP

SEQ ID NO: 70 - protein, 136 aa, synthetic construct
  REGION 1..136: note = Synthetic polymer.
QVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEP

SEQ ID NO: 71 - protein, 235 aa, synthetic construct; identical to SEQ ID NO: 62
  REGION 1..235: note = Synthetic polymer.

SEQ ID NO: 72 - protein, 112 aa, synthetic construct
  REGION 1..112: note = Synthetic polymer.
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 73 - protein, 143 aa, synthetic construct
  REGION 1..143: note = Synthetic polymer.
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQ

SEQ ID NO: 74 - protein, 133 aa, synthetic construct
  REGION 1..133: note = Synthetic polymer.
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 75 - protein, 205 aa, synthetic construct
  REGION 1..205: note = Synthetic polymer.
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 76 - protein, 181 aa, synthetic construct
  REGION 1..181: note = Synthetic polymer.
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 77 - protein, 215 aa, synthetic construct
  REGION 1..215: note = Synthetic polymer.
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 78 - protein, 242 aa, synthetic construct
  REGION 1..242: note = Synthetic polymer.
VRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIG

SEQ ID NO: 79 - protein, 343 aa, synthetic construct
  REGION 1..343: note = Synthetic polymer.
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 80 - protein, 482 aa, synthetic construct
  REGION 1..482: note = Synthetic polymer.
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 81 - protein, 536 aa, synthetic construct
  REGION 1..536: note = Synthetic polymer.
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 82 - protein, 655 aa, synthetic construct
  REGION 1..655: note = Synthetic polymer.
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDTMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 83 - protein, 604 aa, synthetic construct
  REGION 1..604: note = Synthetic polymer.
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 84 - protein, 737 aa, synthetic construct
  REGION 1..737: note = Synthetic polymer.
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDPRFMGTISDVKTNVQLLKGRVDNISTLDSEIKKNSDGMEAAGVQIQMVNESLGYVRSQFLKLKTSVEKANAQIQILTRSWEEVSTLNAQIPELKSDLEKASALNTKIRALQGSLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEP

SEQ ID NO: 85 - protein, 609 aa, synthetic construct
  REGION 1..609: note = Synthetic polymer.
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDQVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEP

SEQ ID NO: 86 - protein, 819 aa, synthetic construct
  REGION 1..819: note = Synthetic polymer.
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 87 - protein, 598 aa, synthetic construct
  REGION 1..598: note = Synthetic polymer.
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICK

SEQ ID NO: 88 - protein, 641 aa, synthetic construct
  REGION 1..641: note = Synthetic polymer.
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDTYHFTYGETGKRLSELHSYHSSLTCFSEGTKVPAWGCCPASWKSFGSSCYFISSEEKVWSKSEQNCVEMGAHLVVFNTEAEQNFIVQQLNESFSYFLGLSDPQGNNNWQWIDKTPYEKNVRFWHLGEPNHSAEQCASIVFWKPTGWGWNDVICETRRNSICEMNKIYL

SEQ ID NO: 89 - protein, 581 aa, synthetic construct
  REGION 1..581: note = Synthetic polymer.
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 90 - protein, 510 aa, synthetic construct
  REGION 1..510: note = Synthetic polymer.
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQSKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMDQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 91 - protein, 630 aa, synthetic construct
  REGION 1..630: note = Synthetic polymer.
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 92 - protein, 681 aa, synthetic construct
  REGION 1..681: note = Synthetic polymer.
VRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 93 - protein, 793 aa, synthetic construct
  REGION 1..793: note = Synthetic polymer.
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

(19) United States
(12) Patent Application Publication
(10) Pub. No.: US 2026/0158108 A1
(43) Pub. Date: Jun. 11, 2026

(54) MODIFIED MRNA THERAPEUTICS

(71) Applicant: Shattuck Labs, Inc., Austin, TX (US)

(72) Inventors: Taylor SCHREIBER, Austin, TX (US); George FROMM, Austin, TX (US); Casey SHUPTRINE, Austin, TX (US)

(21) Appl. No.: 18/706,085

(22) PCT Filed: Nov. 1, 2022
(86) PCT No.: PCT/US2022/079052

§ 371 Date: Apr. 30, 2024

Related U.S. Application Data
(60) Provisional application No. 63/274,232, filed on Nov. 1, 2021.
(60) Provisional application No. 63/320,628, filed on Mar. 16, 2022.
(60) Provisional application No. 63/325,568, filed on Mar. 30, 2022.
(60) Provisional application No. 63/369,836, filed on Jul. 29, 2022.

(51) Int. Cl. A61K 38/17; A61K 9/00; A61K 9/51; A61K 31/7105; A61K 31/7115; A61K 38/19; A61P 35/00

(52) U.S. Cl. CPC A61K 38/1774; A61K 9/0019; A61K 9/5123; A61K 31/7105; A61K 31/7115; A61K 38/191; A61P 35/00

(57) ABSTRACT

The present disclosure relates to, inter alia, compositions and methods for nucleic acid-based delivery of chimeric proteins that find use in the treatment of a disease or disorder such as selected from cancer, autoimmunity, fibrotic disease, and other inflammatory disorders.

### PRIORITY

This application claims the benefit of, and priority to, U.S. Provisional Application No. 63/274,232, filed Nov. 1, 2021, U.S. Provisional Application No. 63/320,628, filed Mar. 16, 2022, U.S. Provisional Application No. 63/325,568, filed Mar. 30, 2022, and U.S. Provisional Application No. 63/369,836, filed Jul. 29, 2022, the contents of each of which are hereby incorporated by reference in their entirety.

### TECHNICAL FIELD

The present disclosure relates to, inter alia, compositions and methods for delivering nucleic acids encoding chimeric proteins that find use in the treatment of a disease or disorder, such as immunotherapies for cancer, autoimmunity, and inflammatory disorders.

### SEQUENCE LISTING

The instant application contains a sequence listing, which has been submitted in XML format via Patent Center. The contents of the XML copy named “SHK-061PC_116981-5054_Sequence Listing”, which was created on Oct. 31, 2022 and is 130,810 bytes in size, are incorporated herein by reference in their entirety.

### BACKGROUND

Therapeutic proteins provide an opportunity to treat a wide range of indications, including cancer and inflammatory and autoimmune disorders. There are a wide variety of therapeutic proteins, including antibodies and recombinant fusion proteins, that continue to be developed for administration as proteins. In some cases, these proteins can demonstrate rapid clearance from circulation and/or limited serum half-life, and this may lead to sub-therapeutic concentrations and/or a limited duration of action. As a result, therapeutics proteins often require repeated, frequent, or prolonged administration.

The field of nucleic acid therapeutics has grown tremendously over the past several years. Nucleic acid therapeutics can achieve long-lasting or even permanent therapeutic effects via, e.g., gene provision, replacement, or editing. However, their clinical applications have been limited despite decades of research and development efforts, mainly because the problems associated with the delivery of nucleic acid therapeutics. Recent events suggest that certain nucleic acid therapeutics may be successful when gene provision is desired. For example, mRNA vaccines and adenovirus-based vaccines have proven successful against infectious diseases such as Covid-19. Moreover, certain viral vector-based therapeutics, e.g., alipogene tiparvovec (GLYBERA, uniQure), voretigene neparvovec-rzyl (LUXTURNA, Spark Therapeutics), and onasemnogene abeparvovec (ZOLGENSMA, AveXis/Novartis) have been approved for the treatment of certain hereditary disorders.

Accordingly, there is a need to adapt nucleic acid delivery strategies to provide alternative delivery mechanisms for therapeutic proteins to provide more convenient and/or more effective treatment.

### SUMMARY

Accordingly, in various aspects, the present disclosure provides nucleic acid-based therapeutic compositions and methods that are useful, inter alia, in the treatment of various diseases or disorders (without limitation, e.g., cancer, autoimmunity, fibrotic disease, and other inflammatory disorders).

In one aspect, the present disclosure relates to a method for treating a cancer, an autoimmune condition, or an inflammatory disorder subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of T cell immunoreceptor with Ig and ITIM domains (TIGIT), or a variant or a fragment thereof that is capable of binding a TIGIT ligand, (c) is a second domain comprising an extracellular domain of glucocorticoid-induced TNFR-related protein ligand (GITRL), or a variant or a fragment thereof that is capable of binding a GITRL receptor, or LIGHT (homologous to lymphotoxin, exhibits inducible expression and competes with HSV glycoprotein D for binding to herpesvirus entry mediator, a receptor expressed on T lymphocytes), or a variant or a fragment thereof that is capable of binding a LIGHT receptor, (b) is a linker adjoining the first and second domains, wherein the linker comprises at least one cysteine residue capable of forming a disulfide bond and/or comprises a hinge-CH2-CH3 Fc domain. In embodiments, the TIGIT ligand is selected from CD155/PVR, Nectin-2, Nectin-3 and Nectin-4. In embodiments, the GITRL receptor is glucocorticoid-induced TNFR-related protein (GITR). In embodiments, the LIGHT receptor is TNFRSF3/LTBR.

In one aspect, the present disclosure relates to a method for treating a cancer, an autoimmune condition, or an inflammatory disorder subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of programmed death-1 (PD-1), or a variant or a fragment thereof that capable of binding a PD-1 ligand, (c) is a second domain comprising an extracellular domain of OX40 ligand (OX40L), or a variant or a fragment thereof that capable of binding an OX40L receptor, or 4-1BB Ligand (4-1BBL), or a variant or a fragment thereof that capable of binding a 4-1BBL receptor, (b) is a linker adjoining the first and second domains, wherein the linker comprises at least one cysteine residue capable of forming a disulfide bond and/or comprises a hinge-CH2-CH3 Fc domain. In embodiments, the PD-1 ligand is PD-L1 or PD-L2. In embodiments, the OX40L receptor is OX40. In embodiments, the 4-1BBL receptor is 4-1BB.

In one aspect, the present disclosure relates to a method for treating a cancer, an autoimmune condition, or an inflammatory disorder subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of T-cell immunoglobulin mucin receptor 3 (TIM3), or a variant or a fragment thereof that capable of binding a TIM3 ligand, (c) is a second domain comprising an extracellular domain of CD40 ligand (CD40L), or a variant or a fragment thereof that capable of binding a CD40L receptor, (b) is a linker adjoining the first and second domains, wherein the linker comprises at least one cysteine residue capable of forming a disulfide bond and/or comprises a hinge-CH2-CH3 Fc domain. In embodiments, the TIM3 ligand is selected from galectin 9, phosphatidylserine (PtdSer), CEACAM1 and high mobility group protein B1 (HMGB1). In embodiments, the CD40L receptor is CD40.

In one aspect, the present disclosure relates to a method for treating a cancer, an autoimmune condition, or an inflammatory disorder subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of V-set and immunoglobulin domain-containing protein 8 (VSIG8), or a variant or a fragment thereof that capable of binding a VSIG8 ligand, (c) is a second domain comprising an extracellular domain of 4-1BB Ligand (4-1BBL), or a variant or a fragment thereof that capable of binding a 4-1BBL receptor, (b) is a linker adjoining the first and second domains, wherein the linker comprises at least one cysteine residue capable of forming a disulfide bond and/or comprises a hinge-CH2-CH3 Fc domain. In embodiments, the 4-1BBL receptor is 4-1BB.

In one aspect, the present disclosure relates to a pharmaceutical composition comprising an isolated modified mRNA (mmRNA) encoding a heterologous chimeric protein having an amino acid sequence that has at least about 95% sequence identity with an amino acid sequence selected from SEQ ID NOs: 80-93.

In embodiments, the mmRNA further comprises a 3′ untranslated region (UTR). In embodiments, the 3′ UTR comprises at least one microRNA-122 (miR-122) binding site. In embodiments, the miR-122 binding site is a miR-122-3p binding site or a miR-122-5-binding site. In embodiments, the mmRNA further comprises a spacer sequence between the open reading frame and the miRNA binding site. In embodiments, the spacer sequence comprises at least about 10 nucleotides, at least about 20 nucleotides, at least about 30 nucleotides, at least about 40 nucleotides, at least about 50 nucleotides, at least about 60 nucleotides, at least about 70 nucleotides, at least about 80 nucleotides, at least about 90 nucleotides, or at least about 100 nucleotides.

In embodiments, the mmRNA further comprises a 5′ UTR. In embodiments, the 5′ UTR harbors a Kozak sequence and/or forms a secondary structure that stimulate elongation factor binding.

In embodiments, the mmRNA further comprises a 5′ terminal cap. In embodiments, the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

In any of the embodiments disclosed herein, the mmRNA may comprise one or more modifications. In any of the embodiments disclosed herein, the mmRNA may comprise at least one modification. In embodiments, the modification is nucleoside modification. In embodiments, the modification is a base modification. In embodiments, the modification is a sugar-phosphate backbone modification.

In embodiments, the modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and a combination of any two or more thereof. In embodiments, the modifications are selected from pseudouridine (Ψ), N1-methylpseudouridine (m1Ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1Ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methylinosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and a combination of any two or more thereof. In embodiments, modification is selected from pseudouridine, N1-methylpseudouridine, 5-methylcytosine, 5-methoxyuridine, and a combination thereof.

In embodiments, the mmRNA comprises at least one N1-methylpseudouridine. In embodiments, the mmRNA is fully modified with chemically-modified uridines. In embodiments, the mmRNA is a fully modified with N1-methylpseudouridine.

In embodiments, the modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine or a combination of any two or more thereof.

In embodiments, the modifications are selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In embodiments, the modifications are selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In embodiments, the modifications are selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In embodiments, the modifications are present on the major groove face. In embodiments, a hydrogen on C-5 of uracil is replaced with a methyl group or a halo group.

In embodiments, the mmRNA further comprises one or more modifications selected from 5′-O-(1-Thiophosphate)-Adenosine, 5′-O-(1-Thiophosphate)-Cytidine, 5′-O-(1-Thiophosphate)-Guanosine, 5′-O-(1-Thiophosphate)-Uridine and 5′-O-(1-Thiophosphate)-Pseudouridine.

In any of the embodiments disclosed herein, the pharmaceutical composition may further comprise a lipidoid, a liposome, a lipoplex, a lipid nanoparticle, a polymeric nanoparticle, a peptide, a protein, a cell, a nanoparticle mimic, a nanotube, or a conjugate. In embodiments, the pharmaceutical composition is formulated as a lipid nanoparticle (LNP), a lipoplex, or a liposome.

In embodiments, the pharmaceutical composition is formulated as a lipid nanoparticle (LNP). In embodiments, the LNP comprises a molar ratio of about 20-60% ionizable amino lipid, about 5-25% phospholipid, about 25-55% structural lipid, and about 0.5-1.5% PEG lipid. In embodiments, the LNP comprises a molar ratio of about 50% ionizable amino lipid, about 8-12% phospholipid, about 37-40% structural lipid, and about 1-2% PEG lipid. In embodiments, the lipid nanoparticles comprise lipids selected from an ionizable lipid (e.g., an ionizable cationic lipid selected from DLin-DMA, DLin-K-DMA, DLin-KC2-DMA, DLin-MC3-DMA, 98N12-5, and C12-200); a structural lipid (e.g., distearoylphosphatidylcholine (DSPC)); cholesterol, and a polyethyleneglycol (PEG)-lipid (e.g., a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); dioleoylphosphatidylethanolamine (DOPE). In embodiments, the lipid nanoparticles comprise (a) a cationic lipid comprising from 50 mol % to 85 mol % of the total lipid present in the particle; (b) a non-cationic lipid comprising from 13 mol % to 49.5 mol % of the total lipid present in the particle; and (c) a conjugated lipid that inhibits aggregation of particles comprising from 0.5 mol % to 2 mol % of the total lipid present in the particle. In embodiments, the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

In any of the embodiments disclosed herein, the pharmaceutical composition is formulated for parenteral administration. In any of the embodiments disclosed herein, the pharmaceutical composition is formulated for topical administration.

In one aspect, the present disclosure relates to a method for inducing lymphocyte margination in a human subject in need thereof, the method comprising a step of administering to the subject a pharmaceutical composition comprising an isolated polynucleotide encoding a chimeric protein having a general structure of: N terminus—(a)-(b)-(c)—C terminus. In embodiments, the isolated polynucleotide is the isolated polynucleotide of any of the embodiments disclosed herein.

### BRIEF DESCRIPTION OF THE DRAWINGS

FIG. 1A shows schematic illustrations of Type I transmembrane proteins (left protein) and Type II transmembrane proteins (right protein). FIG. 1B shows two membrane-anchored extracellular proteins, with the curved lines represents the anchoring domains; the left protein has its carboxy terminus anchored to the cell membrane and the right protein has its amino terminus anchored to the cell membrane. FIG. 1C shows two secreted proteins (which lack a transmembrane domain or a membrane anchorage); the left protein has its ligand/receptor binding site at it amino terminus (“N-”) and the right protein has its ligand/receptor binding site at its carboxy terminus (“C-”). FIG. 1D and FIG. 1E show illustrations of chimeric proteins of the present disclosure; there, linkers connect the two extracellular binding domains.

FIG. 2 shows the expression of the TIGIT-Fc-GITRL chimeric protein in serum by HEK293 (human embryonic kidney cells) contacted with a lipid nanoparticle (LNP) comprising a modified mRNA (mmRNA) encoding the murine TIGIT-Fc-GITRL chimeric protein.

FIG. 3 shows the pharmacokinetics of the TIGIT-Fc-GITRL chimeric protein in serum of mice injected with 50 μg of the mouse TIGIT-Fc-GITRL chimeric protein or 3.5 μg of an LNP comprising an mmRNA encoding the TIGIT-Fc-GITRL chimeric protein. A fold-change in serum concentrations of the TIGIT-Fc-GITRL chimeric protein from each group at 24 or 48 hours compared to the 4-hour time point is plotted.

FIG. 4A and FIG. 4B demonstrate the expression of the TIGIT-Fc-GITRL chimeric protein in tissues. FIG. 4A shows the expression of the TIGIT-Fc-GITRL chimeric protein in spleen and liver of mice injected with 3.5 μg of an LNP comprising an mmRNA encoding the TIGIT-Fc-GITRL chimeric protein. FIG. 4B shows the quantitation of the TIGIT-Fc-GITRL chimeric protein in spleen and liver of mice injected with 50 μg of the TIGIT-Fc-GITRL chimeric protein.

FIG. 5A to FIG. 5C demonstrate the expression of cytokines in response to the delivery of modified mRNA (mmRNA)-based delivery of the TIGIT-Fc-GITRL chimeric protein. Mice were administered 3.5 μg of an LNP comprising an mmRNA encoding the mouse TIGIT-Fc-GITRL chimeric protein (indicated as “mRNA”), empty LNP lacking mmRNA (“LNP”), or 50 μg of the mouse TIGIT-Fc-GITRL chimeric protein (“fFP”). The empty LNP and the mouse TIGIT-Fc-GITRL chimeric protein served as negative control and positive control, respectively. Serum was collected at 4, 24, 48 and 72 hours post the treatments and the amount of IFNγ (FIG. 5A), MIP-3a (FIG. 5B), and TNFα (FIG. 5C) were quantitated and plotted. Dotted lines are drawn at the level of the highest signal achieved with the negative control (empty LNP).

FIG. 6A to FIG. 6T demonstrate the target immune cell margination induced in response to the delivery of modified mRNA (mmRNA)-based delivery of the SIRPα-Fc-CD40L or TIGIT-Fc-LIGHT chimeric proteins. The percent abundance of total CD8+ cells compared to total CD45+CD3+ cells at 3 hours (FIG. 6A) and 24 hours post-dosing (FIG. 6B), activated CD8+CD69+ T cells compared to total CD45+CD3+ cells at 3 hours (FIG. 6C) and 24 hours post-dosing (FIG. 6D), and total CD4+ cells compared to total CD45+CD3+ cells at 3 hours (FIG. 6E) and 24 hours post-dosing (FIG. 6F) is shown. Also shown is the percent abundance of total NKP46+ Natural Killer cells (NK cells) compared to total CD45+CD3− cells at 3 hours (FIG. 6G) and 24 hours post-dosing (FIG. 6H), and activated NKP46+CD69+ NK cells compared to total CD45+CD3− cells at 3 hours (FIG. 6I) and 24 hours post-dosing (FIG. 6J); CD20+ B Cells compared to total CD45+CD3− cells at 3 hours (FIG. 6K) and 24 hours post-dosing (FIG. 6L); total CD11b+ Antigen Presenting Cells compared to total CD45+CD3− cells at 3 hours (FIG. 6M) and 24 hours post-dosing (FIG. 6N), and activated CD11b+CD80+ Antigen Presenting Cells compared to total CD45+CD3− cells at 3 hours (FIG. 6O) and 24 hours post-dosing (FIG. 6P); total CD11c+ Antigen Presenting Cells compared to total CD45+CD3− cells at 3 hours (FIG. 6Q) and 24 hours post-dosing (FIG. 6R), and activated CD11c+CD80+ Antigen Presenting Cells compared to total CD45+CD3− cells at 3 hours (FIG. 6S) and 24 hours post-dosing (FIG. 6T). Statistical Significance was determined by one-way ANOVA with Multiple Comparisons vs. vehicle only-treated group (or as indicated by the brackets). * denotes p<0.05; ** denotes p<0.01, *** denotes p<0.001, and **** denotes p<0.0001.

FIG. 7A to FIG. 7B demonstrate the in vivo changes in activated lymphocytes induced by modified mRNA (mmRNA) encoding the SIRPα-Fc-CD40L or TIGIT-Fc-LIGHT chimeric proteins. Tumor-bearing mice were administered empty LNP lacking mmRNA (“vehicle (LNP)”), 200 μg of the purified recombinant mouse SIRPα-Fc-CD40L or TIGIT-Fc-LIGHT chimeric proteins, or LNP comprising 12.5 μg or 25 μg of modified mRNA (mmRNA) encoding the mouse SIRPα-Fc-CD40L or TIGIT-Fc-LIGHT chimeric proteins. FIG. 7A shows a line graph of the percentage of activated CD8+CD69+ T cells (out of CD45+CD3+) in tumor-draining lymph nodes (TDLN) as a function of time after the treatments. The dotted line shows the percentage of activated CD8+CD69+ T cells (out of CD45+CD3+) in TDLN of vehicle (LNP)-treated mice at 168 hours. FIG. 7B shows a line graph of the percentage of activated CD8+CD69+ intratumoral T cells (out of CD45+CD3+) as a function of time after the treatments. Dotted lines are drawn at the level of the highest signal achieved with the negative control (empty LNP). The dotted line shows the percentage of activated CD8+CD69+ T cells (out of CD45+CD3+) in the tumors of vehicle (LNP)-treated mice at 168 hours.

FIG. 8 shows the in vivo anti-tumor efficacy of modified mRNA (mmRNA) encoding the SIRPα-Fc-CD40L or TIGIT-Fc-LIGHT chimeric proteins. Tumor-bearing mice were administered empty LNP lacking mmRNA (“vehicle (LNP)”), 200 μg of the purified recombinant mouse SIRPα-Fc-CD40L or TIGIT-Fc-LIGHT chimeric proteins, or LNP comprising 12.5 μg or 25 μg of modified mRNA (mmRNA) encoding the mouse SIRPα-Fc-CD40L or TIGIT-Fc-LIGHT chimeric proteins. Mean tumor sizes were measured at 3 days post-treatment and plotted. The dotted line shows the mean tumor volume of vehicle (LNP)-treated mice.

FIG. 9A to FIG. 9C show the in vivo persistence of modified mRNA (mmRNA) encoding the SIRPα-Fc-CD40L chimeric protein. Mice bearing CT26 tumor were administered empty LNP lacking mmRNA (“empty LNP”), 200 μg of the purified recombinant mouse SIRPα-Fc-CD40L chimeric protein, or LNP comprising 12.5 μg of modified mRNA (mmRNA) encoding the mouse SIRPα-Fc-CD40L chimeric protein. Mice were sacrificed at the indicated times and RNA was isolated from liver, spleen and tumor. The RNA was reverse transcribed, and amplified with primers that span the SIRPα and Fc domain (therefore specific to the SIRPα-Fc-CD40L chimeric protein). Beta actin (ACTB) primers were used as a house-keeping gene control. The levels of mmRNA encoding the SIRPα-Fc-CD40L chimeric protein in liver (FIG. 9A), spleen (FIG. 9B) and tumor (FIG. 9C) was determined using the ΔCq method. The dotted line shows the ΔCq of SIRPα-Fc-CD40L mmRNA compared to ACTB mRNA.

FIG. 10A to FIG. 10C show the in vivo persistence of modified mRNA (mmRNA) encoding the TIGIT-Fc-LIGHT chimeric protein. Mice bearing CT26 tumor were administered empty LNP lacking mmRNA (“empty LNP”), 200 μg of the purified recombinant mouse TIGIT-Fc-LIGHT chimeric protein, or LNP comprising 12.5 μg of modified mRNA (mmRNA) encoding the mouse TIGIT-Fc-LIGHT chimeric protein. Mice were sacrificed at the indicated times and RNA was isolated from liver, spleen and tumor. The RNA was reverse transcribed, and amplified with primers that span the TIGIT and Fc domain (therefore specific to the TIGIT-Fc-LIGHT chimeric protein). Beta actin (ACTB) primers were used as a house-keeping gene control. The levels of mmRNA encoding the TIGIT-Fc-LIGHT chimeric protein in liver (FIG. 10A), spleen (FIG. 10B) and tumor (FIG. 10C) was determined using the ΔCq method. The dotted line shows the ΔCq of TIGIT-Fc-LIGHT mmRNA compared to ACTB mRNA.

FIG. 11A to FIG. 11C show the in vivo serum cytokine response induced by modified mRNA (mmRNA) encoding the SIRPα-Fc-CD40L and TIGIT-Fc-LIGHT chimeric proteins, in comparison with the purified TIGIT-Fc-LIGHT and SIRPα-Fc-CD40L chimeric proteins. Mice bearing CT26 tumor were administered empty LNP lacking mmRNA (“empty LNP”), 200 μg of the purified recombinant mouse SIRPα-Fc-CD40L and TIGIT-Fc-LIGHT chimeric proteins, or LNP comprising 12.5 μg of modified mRNA (mmRNA) encoding the mouse SIRPα-Fc-CD40L and TIGIT-Fc-LIGHT chimeric proteins. Blood was collected from the mice at the indicated times and the levels of IP-10 (CXCL10) (FIG. 11A), IFNγ (FIG. 11B) and MCP-1 (CCL2) (FIG. 11C) were plotted.

FIG. 12A and FIG. 12B show the in vivo immune cell activation induced by modified mRNA (mmRNA) encoding the SIRPα-Fc-CD40L and TIGIT-Fc-LIGHT chimeric proteins, in comparison with the purified TIGIT-Fc-LIGHT and SIRPα-Fc-CD40L chimeric proteins. Mice bearing CT26 tumor were administered empty LNP lacking mmRNA (“LNP-Cntrl”), 200 μg of the purified recombinant mouse SIRPα-Fc-CD40L and TIGIT-Fc-LIGHT chimeric proteins, or LNP comprising 12.5 μg of modified mRNA (mmRNA) encoding the mouse SIRPα-Fc-CD40L and TIGIT-Fc-LIGHT chimeric proteins. The mice were sacrificed at the indicated times and spleens were isolated and CD80/CD86+ and CD11c+ were quantitated using flow cytometry. The kinetics of immune cell activation (% CD80/CD86+ of total CD11c+ cells) in mice treated with mmRNA encoding the SIRPα-Fc-CD40L chimeric protein or purified SIRPα-Fc-CD40L chimeric protein in comparison with empty LNP was plotted (FIG. 12A). Similarly, kinetics of immune cell activation in mice treated with mmRNA encoding the TIGIT-Fc-LIGHT chimeric protein or purified TIGIT-Fc-LIGHT chimeric protein in comparison with empty LNP was plotted (FIG. 12B).

FIG. 13 shows the in vivo anti-tumor efficacy of modified mRNA (mmRNA) encoding the SIRPα-Fc-CD40L and TIGIT-Fc-LIGHT chimeric proteins, in comparison with the purified TIGIT-Fc-LIGHT and SIRPα-Fc-CD40L chimeric proteins. Mice were injected with CT26 tumors cells and when average starting tumor volume reached 137.41 mm³ (day 0), the mice were randomly distributed in three treatment groups: empty LNP lacking mmRNA (“LNP only Cntrl”), or LNP comprising 12.5 μg of modified mRNA (mmRNA) encoding the mouse SIRPα-Fc-CD40L or TIGIT-Fc-LIGHT chimeric proteins. Mice were dosed on days 0 and 7. Mean tumor sizes were measured on indicated days and plotted.

FIG. 14A and FIG. 14B show the in vitro nucleic acid delivery of a DNA minicircle co-expressing GFP and the SIRPα-Fc-CD40L chimeric protein. FIG. 14A shows the kinetics of the expression of GFP as assessed using IncuCyte time-lapse microscopy. FIG. 14B shows the expression of the SIRPα-Fc-CD40L chimeric protein from culture supernatant as measured using a dual antibody Meso Scale Discovery (MSD) ELISA assay.

FIG. 15A to FIG. 15C demonstrate the in vivo nucleic acid-based delivery of the SIRPα-Fc-CD40L chimeric protein. FIG. 15A shows the expression of the SIRPα-Fc-CD40L chimeric protein in serum. FIG. 15B shows the expression of the SIRPα-Fc-CD40L chimeric protein in bone marrow. FIG. 15C shows the expression of the SIRPα-Fc-CD40L chimeric protein in dissociated liver tissue.

FIG. 16A to FIG. 16C show the in vivo activity of the SIRPα-Fc-CD40L chimeric protein when delivered in form of a DNA minicircle. FIG. 16A shows the margination of CD20+ IgD+ B cells out of the peripheral blood induced by nucleic acid-based delivery of the SIRPα-Fc-CD40L chimeric protein. FIG. 16B and FIG. 16C show the in vivo the efficacy of a nucleic acid encoding the SIRPα-Fc-CD40L chimeric protein against CT26 allografts. FIG. 16B shows the growth curves of tumors from the mice treated with vehicle only control, or 5, 15 or 30 μg of a DNA minicircle expressing the SIRPα-Fc-CD40L chimeric protein. FIG. 16C shows the tumor volumes from the mice treated with vehicle only control, or 5, 15 or 30 μg of a DNA minicircle expressing the SIRPα-Fc-CD40L chimeric protein.

### DETAILED DESCRIPTION

The present disclosure is based, in part, on the discovery of that certain heterologous chimeric proteins may be delivered in form of a modified mRNA encoding the same, and that such nucleic-acid-based delivery leads to sustained persistence of mmRNA, sustained expression of the in heterologous chimeric proteins resulting in accumulation of the heterologous chimeric proteins over time in serum and in tissues. The present disclosure is also based, in part, on the discovery of that, a modified mRNA-based delivery of the chimeric proteins leads to sustained production of adaptive and innate immune cytokines (e.g., IP-10 (CXCL10), IFNγ, MCP-1 (CCL2), MIP-3a (CCL20) and TNFα). Interestingly, mRNA encoding the present chimeric proteins resulted in equivalent or greater target immune cell margination than the recombinant proteins. In addition, the mmRNA encoding the chimeric proteins of the present disclosure induce rapid and sustained immune stimulation, e.g., featuring the activation of stimulated immune cells (e.g., CD8+CD69+ T cells and NK cells as well as CD80/CD86+CD11c+ splenic dendritic cells), which results in immediate control of tumor growth.

A large number of small molecule therapeutics have been approved. Since early 1980s, a total of 239 therapeutic proteins and peptides are approved for clinical use by US-FDA. The field of nucleic acid therapeutics has grown tremendously over the past several years. Nucleic acid therapeutics can in theory achieve long-lasting or even permanent therapeutic effects via, e.g., gene provision, replacement or editing. However, their clinical applications have been limited vaccines and for the treatment of certain hereditary disorders. The data presented herein demonstrate that nucleic acid-based therapeutics may be helpful in treating other diseases or disorders (without limitation, e.g., cancer, autoimmunity, fibrotic disease, and other inflammatory disorders).

Accordingly, in one aspect, the present disclosure relates to a method for treating a cancer, an autoimmune condition, or an inflammatory disorder subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of T cell immunoreceptor with Ig and ITIM domains (TIGIT), or a variant or a fragment thereof that is capable of binding a TIGIT ligand, (c) is a second domain comprising an extracellular domain of glucocorticoid-induced TNFR-related protein ligand (GITRL), or a variant or a fragment thereof that is capable of binding a GITRL receptor, or LIGHT (homologous to lymphotoxin, exhibits inducible expression and competes with HSV glycoprotein D for binding to herpesvirus entry mediator, a receptor expressed on T lymphocytes), or a variant or a fragment thereof that is capable of binding a LIGHT receptor, (b) is a linker adjoining the first and second domains, wherein the linker comprises at least one cysteine residue capable of forming a disulfide bond and/or comprises a hinge-CH2-CH3 Fc domain. In embodiments, the TIGIT ligand is selected from CD155/PVR, Nectin-2, Nectin-3 and Nectin-4. In embodiments, the GITRL receptor is glucocorticoid-induced TNFR-related protein (GITR). In embodiments, the LIGHT receptor is TNFRSF3/LTBR. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to a method for treating a cancer, an autoimmune condition, or an inflammatory disorder subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of tumor necrosis factor (TNF) receptor 2 (TNFR2), or a variant or a fragment thereof that is capable of binding a TNFR2 ligand, (c) is a second domain comprising an extracellular domain selected from CLEC7a, or a variant or a fragment thereof that capable of binding a CLEC7a ligand, DC-SIGN(CD209), or a variant or a fragment thereof that capable of binding a DC-SIGN(CD209) ligand, DECTIN2(CLEC6A), or a variant or a fragment thereof that capable of binding a DECTIN2(CLEC6A) ligand, Langerin(CD207,CLC4K), or a variant or a fragment thereof that capable of binding a Langerin(CD207,CLC4K) ligand, CD69, or a variant or a fragment thereof that capable of binding a CD69 ligand, and TGF-beta, or a variant or a fragment thereof that capable of binding a TGF-beta receptor, (b) is a linker adjoining the first and second domains, wherein the linker comprises at least one cysteine residue capable of forming a disulfide bond and/or comprises a hinge-CH2-CH3 Fc domain. In embodiments, the TNFR2 ligand is TNFα. In embodiments, the CLEC7a ligand is a beta-1,3-linked and/or beta-1,6-linked glucan. In embodiments, the DC-SIGN(CD209) ligand is a Intercellular Adhesion Molecule 2 (ICAM2) and/or Intercellular Adhesion Molecule 3 (ICAM3). In embodiments, the DECTIN2(CLEC6A) ligand is an alpha-mannan. In embodiments, the Langerin(CD207,CLC4K) ligand is a sulfated glycan, a mannosylated glycan, a keratan sulfate (KS) and/or a beta-glucan. In embodiments, the CD69 ligand is Galectin-1 (Gal-1) or the S100A8/S100A9 complex. In embodiments, the TGF-beta receptor is TGFBR1 and TGFBR2. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to a method for treating a cancer, an autoimmune condition, or an inflammatory disorder subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of programmed death-1 (PD-1), or a variant or a fragment thereof that capable of binding a PD-1 ligand, (c) is a second domain comprising an extracellular domain of OX40 ligand (OX40L), or a variant or a fragment thereof that capable of binding an OX40L receptor, or 4-1BB Ligand (4-1BBL), or a variant or a fragment thereof that capable of binding a 4-1BBL receptor, (b) is a linker adjoining the first and second domains, wherein the linker comprises at least one cysteine residue capable of forming a disulfide bond and/or comprises a hinge-CH2-CH3 Fc domain. In embodiments, the PD-1 ligand is PD-L1 or PD-L2. In embodiments, the OX40L receptor is OX40. In embodiments, the 4-1BBL receptor is 4-1BB. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to a method for treating a cancer, an autoimmune condition, or an inflammatory disorder subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of T-cell immunoglobulin mucin receptor 3 (TIM3), or a variant or a fragment thereof that capable of binding a TIM3 ligand, (c) is a second domain comprising an extracellular domain of CD40 ligand (CD40L), or a variant or a fragment thereof that capable of binding a CD40L receptor, (b) is a linker adjoining the first and second domains, wherein the linker comprises at least one cysteine residue capable of forming a disulfide bond and/or comprises a hinge-CH2-CH3 Fc domain. In embodiments, the TIM3 ligand is selected from galectin 9, phosphatidylserine (PtdSer), CEACAM1 and high mobility group protein B1 (HMGB1). In embodiments, the CD40L receptor is CD40.

In one aspect, the present disclosure relates to a method for treating a cancer, an autoimmune condition, or an inflammatory disorder subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of V-set and immunoglobulin domain-containing protein 8 (VSIG8), or a variant or a fragment thereof that capable of binding a VSIG8 ligand, (c) is a second domain comprising an extracellular domain of 4-1BB Ligand (4-1BBL), or a variant or a fragment thereof that capable of binding a 4-1BBL receptor, (b) is a linker adjoining the first and second domains, wherein the linker comprises at least one cysteine residue capable of forming a disulfide bond and/or comprises a hinge-CH2-CH3 Fc domain. In embodiments, the 4-1BBL receptor is 4-1BB. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to a method for treating a cancer, an autoimmune condition, or an inflammatory disorder subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of signal regulatory protein a (SIRPα), or a variant or a fragment thereof that is capable of binding a SIRPα ligand, (c) is a second domain comprising an extracellular domain of CD40 ligand (CD40L), or a variant or a fragment thereof that is capable of binding a CD40L receptor, (b) is a linker adjoining the first and second domains, wherein the linker comprises at least one cysteine residue capable of forming a disulfide bond and/or comprises a hinge-CH2-CH3 Fc domain. In embodiments, the SIRPα ligand is CD47. In embodiments, the CD40L receptor is CD40. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to a method for inducing rapid and sustained immune stimulation or immune inhibition subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of T cell immunoreceptor with Ig and ITIM domains (TIGIT), or a variant or a fragment thereof that is capable of binding a TIGIT ligand, (c) is a second domain comprising an extracellular domain of glucocorticoid-induced TNFR-related protein ligand (GITRL), or a variant or a fragment thereof that is capable of binding a GITRL receptor, or LIGHT (homologous to lymphotoxin, exhibits inducible expression and competes with HSV glycoprotein D for binding to herpesvirus entry mediator, a receptor expressed on T lymphocytes), or a variant or a fragment thereof that is capable of binding a LIGHT receptor, (b) is a linker adjoining the first and second domains, wherein the linker comprises at least one cysteine residue capable of forming a disulfide bond and/or comprises a hinge-CH2-CH3 Fc domain. In embodiments, the TIGIT ligand is selected from CD155/PVR, Nectin-2, Nectin-3 and Nectin-4. In embodiments, the GITRL receptor is glucocorticoid-induced TNFR-related protein (GITR). In embodiments, the LIGHT receptor is TNFRSF3/LTBR. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to a method for inducing rapid and sustained immune inhibition subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of tumor necrosis factor (TNF) receptor 2 (TNFR2), or a variant or a fragment thereof that is capable of binding a TNFR2 ligand, (c) is a second domain comprising an extracellular domain selected from CLEC7a, or a variant or a fragment thereof that capable of binding a CLEC7a ligand, DC-SIGN(CD209), or a variant or a fragment thereof that capable of binding a DC-SIGN(CD209) ligand, DECTIN2(CLEC6A), or a variant or a fragment thereof that capable of binding a DECTIN2(CLEC6A) ligand, Langerin(CD207,CLC4K), or a variant or a fragment thereof that capable of binding a Langerin(CD207,CLC4K) ligand, CD69, or a variant or a fragment thereof that capable of binding a CD69 ligand, and TGF-beta, or a variant or a fragment thereof that capable of binding a TGF-beta receptor, (b) is a linker adjoining the first and second domains, wherein the linker comprises at least one cysteine residue capable of forming a disulfide bond and/or comprises a hinge-CH2-CH3 Fc domain. In embodiments, the TNFR2 ligand is TNFα. In embodiments, the CLEC7a ligand is a beta-1,3-linked and/or beta-1,6-linked glucan. In embodiments, the DC-SIGN(CD209) ligand is a Intercellular Adhesion Molecule 2 (ICAM2) and/or Intercellular Adhesion Molecule 3 (ICAM3). In embodiments, the DECTIN2(CLEC6A) ligand is an alpha-mannan. In embodiments, the Langerin(CD207,CLC4K) ligand is a sulfated glycan, a mannosylated glycan, a keratan sulfate (KS) and/or a beta-glucan. In embodiments, the CD69 ligand is Galectin-1 (Gal-1) or the S100A8/S100A9 complex. In embodiments, the TGF-beta receptor is TGFBR1 and TGFBR2. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to a method for inducing rapid and sustained immune stimulation or immune inhibition subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of programmed death-1 (PD-1), or a variant or a fragment thereof that capable of binding a PD-1 ligand, (c) is a second domain comprising an extracellular domain of OX40 ligand (OX40L), or a variant or a fragment thereof that capable of binding an OX40L receptor, or 4-1BB Ligand (4-1BBL), or a variant or a fragment thereof that capable of binding a 4-1BBL receptor, (b) is a linker adjoining the first and second domains, wherein the linker comprises at least one cysteine residue capable of forming a disulfide bond and/or comprises a hinge-CH2-CH3 Fc domain. In embodiments, the PD-1 ligand is PD-L1 or PD-L2. In embodiments, the OX40L receptor is OX40. In embodiments, the 4-1BBL receptor is 4-1BB. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to a method for inducing rapid and sustained immune stimulation or immune inhibition subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of T-cell immunoglobulin mucin receptor 3 (TIM3), or a variant or a fragment thereof that capable of binding a TIM3 ligand, (c) is a second domain comprising an extracellular domain of CD40 ligand (CD40L), or a variant or a fragment thereof that capable of binding a CD40L receptor, (b) is a linker adjoining the first and second domains, wherein the linker comprises at least one cysteine residue capable of forming a disulfide bond and/or comprises a hinge-CH2-CH3 Fc domain. In embodiments, the TIM3 ligand is selected from galectin 9, phosphatidylserine (PtdSer), CEACAM1 and high mobility group protein B1 (HMGB1). In embodiments, the CD40L receptor is CD40.

In one aspect, the present disclosure relates to a method for inducing rapid and sustained immune stimulation or immune inhibition subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of V-set and immunoglobulin domain-containing protein 8 (VSIG8), or a variant or a fragment thereof that capable of binding a VSIG8 ligand, (c) is a second domain comprising an extracellular domain of 4-1BB Ligand (4-1BBL), or a variant or a fragment thereof that capable of binding a 4-1BBL receptor, (b) is a linker adjoining the first and second domains, wherein the linker comprises at least one cysteine residue capable of forming a disulfide bond and/or comprises a hinge-CH2-CH3 Fc domain. In embodiments, the 4-1BBL receptor is 4-1BB. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to a method for inducing rapid and sustained immune stimulation or immune inhibition subject comprising a step of administering to the subject a pharmaceutical composition comprising an isolated polynucleotide encoding a chimeric protein having a general structure of: N terminus—(a)-(b)-(c)—C terminus, wherein: (a) is a first domain comprising an extracellular domain of signal regulatory protein a (SIRPα), or a variant or a fragment thereof that is capable of binding a SIRPα ligand, (c) is a second domain comprising an extracellular domain of CD40 ligand (CD40L), or a variant or a fragment thereof that is capable of binding a CD40L receptor, (b) is a linker adjoining the first and second domains, wherein the linker comprises at least one cysteine residue capable of forming a disulfide bond and/or comprises a hinge-CH2-CH3 Fc domain. In embodiments, the SIRPα ligand is CD47. In embodiments, the CD40L receptor is CD40. In embodiments, the isolated polynucleotide is or comprises an mRNA. In embodiments, the isolated polynucleotide is or comprises an mRNA that is modified according to any of the embodiments disclosed herein.

### First and Second Domains of the Chimeric Proteins and Isolated Polynucleotide Coding the Chimeric Proteins

In one aspect, the present disclosure relates to an isolated polynucleotide encoding a chimeric protein having a general structure of: N terminus—(a)-(b)-(c)—C terminus, wherein: (a) is a first domain comprising an extracellular domain of T cell immunoreceptor with Ig and ITIM domains (TIGIT), or a variant or a fragment thereof that is capable of binding a TIGIT ligand, (c) is a second domain comprising an extracellular domain of glucocorticoid-induced TNFR-related protein ligand (GITRL), or a variant or a fragment thereof that is capable of binding a GITRL receptor, or LIGHT (homologous to lymphotoxin, exhibits inducible expression and competes with HSV glycoprotein D for binding to herpesvirus entry mediator, a receptor expressed on T lymphocytes), or a variant or a fragment thereof that is capable of binding a LIGHT receptor, (b) is a linker adjoining the first and second domains, wherein the linker comprises at least one cysteine residue capable of forming a disulfide bond and/or comprises a hinge-CH2-CH3 Fc domain. In embodiments, the TIGIT ligand is selected from CD155/PVR, Nectin-2, Nectin-3 and Nectin-4. In embodiments, the GITRL receptor is glucocorticoid-induced TNFR-related protein (GITR). In embodiments, the LIGHT receptor is TNFRSF3/LTBR. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein having a general structure of: N terminus—(a)-(b)-(c)—C terminus, wherein: (a) is a first domain comprising an extracellular domain of tumor necrosis factor (TNF) receptor 2 (TNFR2), or a variant or a fragment thereof that is capable of binding a TNFR2 ligand, (c) is a second domain comprising an extracellular domain selected from CLEC7a, or a variant or a fragment thereof that capable of binding a CLEC7a ligand, DC-SIGN(CD209), or a variant or a fragment thereof that capable of binding a DC-SIGN(CD209) ligand, DECTIN2(CLEC6A), or a variant or a fragment thereof that capable of binding a DECTIN2(CLEC6A) ligand, Langerin(CD207,CLC4K), or a variant or a fragment thereof that capable of binding a Langerin(CD207,CLC4K) ligand, CD69, or a variant or a fragment thereof that capable of binding a CD69 ligand, and TGF-beta, or a variant or a fragment thereof that capable of binding a TGF-beta receptor, (b) is a linker adjoining the first and second domains, wherein the linker comprises at least one cysteine residue capable of forming a disulfide bond and/or comprises a hinge-CH2-CH3 Fc domain. In embodiments, the TNFR2 ligand is TNFα. In embodiments, the CLEC7a ligand is a beta-1,3-linked and/or beta-1,6-linked glucan. In embodiments, the DC-SIGN(CD209) ligand is a Intercellular Adhesion Molecule 2 (ICAM2) and/or Intercellular Adhesion Molecule 3 (ICAM3). In embodiments, the DECTIN2(CLEC6A) ligand is an alpha-mannan. In embodiments, the Langerin(CD207,CLC4K) ligand is a sulfated glycan, a mannosylated glycan, a keratan sulfate (KS) and/or a beta-glucan. In embodiments, the CD69 ligand is Galectin-1 (Gal-1) or the S100A8/S100A9 complex. In embodiments, the TGF-beta receptor is TGFBR1 and TGFBR2. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein having a general structure of: N terminus—(a)-(b)-(c)—C terminus, wherein: (a) is a first domain comprising an extracellular domain of programmed death-1 (PD-1), or a variant or a fragment thereof that capable of binding a PD-1 ligand, (c) is a second domain comprising an extracellular domain of OX40 ligand (OX40L), or a variant or a fragment thereof that capable of binding an OX40L receptor, or 4-1BB Ligand (4-1BBL), or a variant or a fragment thereof that capable of binding a 4-1BBL receptor, (b) is a linker adjoining the first and second domains, wherein the linker comprises at least one cysteine residue capable of forming a disulfide bond and/or comprises a hinge-CH2-CH3 Fc domain. In embodiments, the PD-1 ligand is PD-L1 or PD-L2. In embodiments, the OX40L receptor is OX40. In embodiments, the 4-1BBL receptor is 4-1BB. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein having a general structure of: N terminus—(a)-(b)-(c)—C terminus, wherein: (a) is a first domain comprising an extracellular domain of T-cell immunoglobulin mucin receptor 3 (TIM3), or a variant or a fragment thereof that capable of binding a TIM3 ligand, (c) is a second domain comprising an extracellular domain of CD40 ligand (CD40L), or a variant or a fragment thereof that capable of binding a CD40L receptor, (b) is a linker adjoining the first and second domains, wherein the linker comprises at least one cysteine residue capable of forming a disulfide bond and/or comprises a hinge-CH2-CH3 Fc domain. In embodiments, the TIM3 ligand is selected from galectin 9, phosphatidylserine (PtdSer), CEACAM1 and high mobility group protein B1 (HMGB1). In embodiments, the CD40L receptor is CD40. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein having a general structure of: N terminus—(a)-(b)-(c)—C terminus, wherein: (a) is a first domain comprising an extracellular domain of V-set and immunoglobulin domain-containing protein 8 (VSIG8), or a variant or a fragment thereof that capable of binding a VSIG8 ligand, (c) is a second domain comprising an extracellular domain of 4-1BB Ligand (4-1BBL), or a variant or a fragment thereof that capable of binding a 4-1BBL receptor, (b) is a linker adjoining the first and second domains, wherein the linker comprises at least one cysteine residue capable of forming a disulfide bond and/or comprises a hinge-CH2-CH3 Fc domain. In embodiments, the 4-1BBL receptor is 4-1BB. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein having a general structure of: N terminus—(a)-(b)-(c)—C terminus, wherein: (a) is a first domain comprising an extracellular domain of signal regulatory protein a (SIRPα), or a variant or a fragment thereof that is capable of binding a SIRPα ligand, (c) is a second domain comprising an extracellular domain of CD40 ligand (CD40L), or a variant or a fragment thereof that is capable of binding a CD40L receptor, (b) is a linker adjoining the first and second domains, wherein the linker comprises at least one cysteine residue capable of forming a disulfide bond and/or comprises a hinge-CH2-CH3 Fc domain. In embodiments, the SIRPα ligand is CD47. In embodiments, the CD40L receptor is CD40. In embodiments, the isolated polynucleotide is or comprises an mRNA. In embodiments, the isolated polynucleotide is or comprises an mRNA that is modified according to any of the embodiments disclosed herein.

Transmembrane proteins typically consist of an extracellular domain, one or a series of transmembrane domains, and an intracellular domain. Without wishing to be bound by theory, the extracellular domain of a transmembrane protein is responsible for interacting with a soluble receptor or ligand or membrane-bound receptor or ligand (i.e., a membrane of an adjacent cell). Without wishing to be bound by theory, the trans-membrane domain(s) is responsible for localizing the transmembrane protein to the plasma membrane. Without wishing to be bound by theory, the intracellular domain of a transmembrane protein is responsible for coordinating interactions with cellular signaling molecules to coordinate intracellular responses with the extracellular environment (or visa-versa). Thus, the transmembrane proteins may function as receptors (i.e. initiate signal transduction in response to stimulation by a cognate ligand), ligands (i.e. stimulate signal transduction in the cells harboring a cognate receptor), or both as receptors and ligands (i.e. both stimulate signal transduction response binding of a cognate ligand and initiate signal transduction in the cells harboring a cognate receptor), depending on the context.

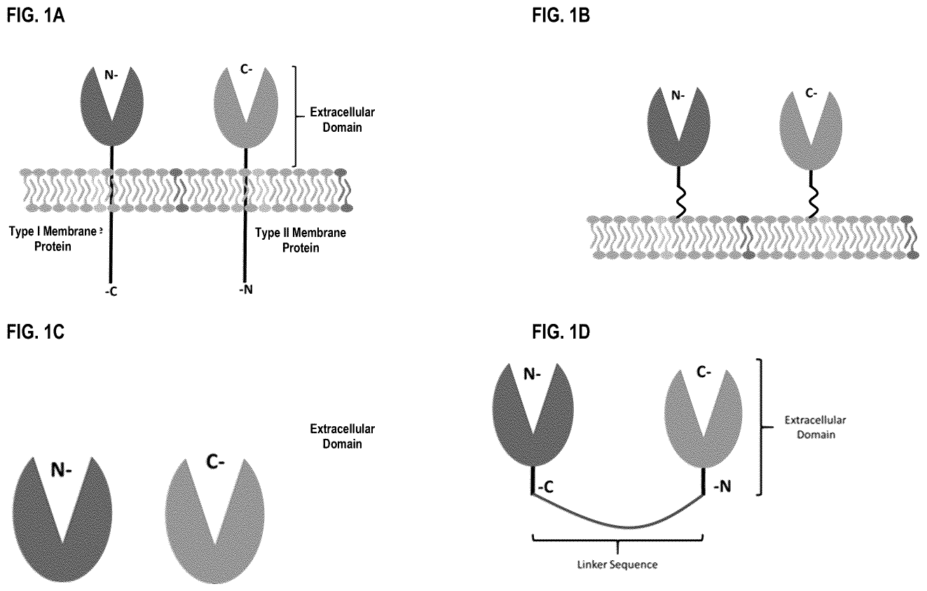

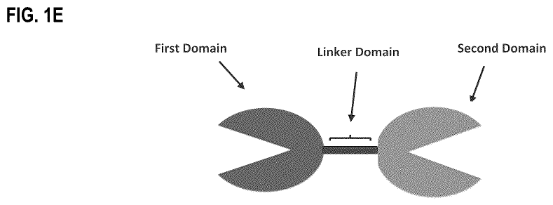

There are generally two types of single-pass transmembrane proteins: Type I transmembrane proteins which have an extracellular amino terminus and an intracellular carboxy terminus (see, FIG. 1A, left protein) and Type II transmembrane proteins which have an extracellular carboxy terminus and an intracellular amino terminus (see, FIG. 1A, right protein). Type I and Type II transmembrane proteins can function as receptors, ligands, or both as receptors and ligands. For Type I transmembrane proteins the amino terminus of the protein faces outside the cell, and therefore contains the functional domains that are responsible for interacting with other binding partners (either ligands or receptors) in the extracellular environment. For Type II transmembrane proteins, the carboxy terminus of the protein faces outside the cell, and therefore contains the functional domains that are responsible for interacting with other binding partners (either ligands or receptors) in the extracellular environment. Thus, these two types of transmembrane proteins have opposite orientations to each other relative to the cell membrane, with the amino terminus of a Type I transmembrane protein is orientated away from the cell membrane whereas the amino terminus of a Type II transmembrane protein is orientated towards from the cell membrane.

In embodiments, an extracellular domain refers to a portion of a transmembrane protein, which is capable of interacting with the extracellular environment. In embodiments, an extracellular domain refers to a portion of a transmembrane protein, which is sufficient for binding to a ligand or receptor and is effective in transmitting a signal to a cell. In embodiments, an extracellular domain is the entire amino acid sequence of a transmembrane protein, which is normally present at the exterior of a cell or of the cell membrane. In embodiments, an extracellular domain is that portion of an amino acid sequence of a transmembrane protein which is external of a cell or of the cell membrane and is needed for signal transduction and/or ligand binding as may be assayed using methods know in the art (e.g., in vitro ligand binding and/or cellular activation assays).

In embodiments, a chimeric protein comprises a portion of a membrane-anchored extracellular protein. Generally, membrane-anchored extracellular protein resides in and interacts with the extracellular environment. In embodiments, the portion of the membrane-anchored extracellular protein is sufficient for binding to a ligand or receptor. In embodiments, the portion is the entire amino acid sequence of the membrane-anchored extracellular protein. Determining whether the portion of the membrane-anchored extracellular protein is capable ligand/receptor binding may be assayed using methods know in the art (e.g., in vitro ligand binding and/or cellular activation assays). FIG. 1B shows two membrane-anchored extracellular proteins, with the curved lines represents the anchoring domains; the left protein has its carboxy terminus anchored to the cell membrane and the right protein has its amino terminus anchored to the cell membrane. Without bound by theory, a membrane-anchored extracellular protein is capable of functioning as a ligand (i.e. stimulating signal transduction in the cells harboring a cognate receptor). FIG. 1C shows two secreted proteins (which lack a transmembrane domain or a membrane anchorage). Some secreted proteins have their ligand/receptor binding site at it amino terminus (“N-”) (FIG. 1C, left) Other secreted proteins have their right protein has its ligand/receptor binding site at its carboxy terminus (“C-”) (FIG. 1C, right).

In chimeric proteins of the present disclosure, a Type I transmembrane protein and a Type II transmembrane protein may be engineered such that their transmembrane and intracellular domains are omitted and the transmembrane proteins' extracellular domains are adjoined using a linker sequence to generate a single chimeric protein. Alternately, two membrane-anchored extracellular proteins may be engineered such that a portion of their extracellular domains are adjoined using a linker sequence to generate a single chimeric protein. Finally, one membrane-anchored extracellular protein and one transmembrane protein (lacking its transmembrane and intracellular domains) may be adjoined using a linker sequence to generate a single chimeric protein. FIG. 1D depicts the linkage of (1) a liberated Type I transmembrane protein (from its transmembrane and intracellular domains) or a liberated carboxy-terminus anchored extracellular protein (from its anchoring domain), or a secreted protein having its ligand/receptor binding site at it amino terminus (“N-”); and (2) a liberated Type II transmembrane protein (from its transmembrane and intracellular domains), or a liberated amino-terminus anchored extracellular protein (from its anchoring domain), or a secreted protein having its ligand/receptor binding site at it carboxy terminus (“C-”), that have been adjoined by (3) a linker sequence. The extracellular domains in this depiction may include the entire amino acid sequence of the Type I protein's extracellular domain or the entire amino acid sequence of the carboxy-anchored extracellular protein, or a fraction thereof, wherein the fraction retains the ability to bind the intended ligand/receptor. Likewise, the extracellular domains in this depiction may include the entire amino acid sequence of the Type II protein's extracellular domain or the entire amino acid sequence of the amino-anchored extracellular protein, or a fraction thereof, wherein the fraction retains the ability to bind the intended ligand/receptor. Likewise, the secreted proteins in this depiction may include the entire amino acid sequence of the secreted proteins, or a fraction thereof, wherein the fraction retains the ability to bind the intended ligand/receptor. Moreover, the chimeric protein comprises sufficient overall flexibility and/or physical distance between domains such that a first extracellular domain (shown at the left end of the chimeric protein in FIG. 1D and FIG. 1E) is sterically capable of binding its receptor/ligand and/or a second extracellular domain (shown at the right end of the chimeric protein in FIG. 1D and FIG. 1E) is sterically capable of binding its receptor/ligand. FIG. 1D and FIG. 1E depict adjoined extracellular domains in a linear chimeric protein wherein each domain comprising an extracellular domain or secreted protein of the chimeric protein is facing “outward.”

Importantly, since a chimeric protein of the present disclosure disrupts, blocks, reduces, inhibits, and/or sequesters the transmission of immunosuppressive signals with one domain, and also either (i) the reception of immunosuppressive signals or (ii) provide an immune stimulatory signal with the other domain, it can provide an anti-tumor effect and/or an antiviral effect by two distinct pathways; this dual-action is more likely to provide a therapeutic effect in a patient and/or to provide an enhanced therapeutic effect in a patient. In embodiments, the linker is not a single amino acid linker, e.g., without limitation, the linker is greater than one amino acid long. In embodiments, the linker has a length of greater than 1-6 amino acids, e.g., without limitation, the linker is greater than seven amino acids long. In embodiments, the linker comprises more than a single glycine residue. Furthermore, since such chimeric proteins can act via two distinct pathways, they can be efficacious, at least, in patients who respond poorly to treatments that target one of the two pathways. Thus, a patient who is a poor responder to treatments acting via one of the two pathways can receive a therapeutic benefit by targeting the other pathway.

In some embodiments, an isolated polynucleotide (without limitations, e.g., modified mRNA) encoding chimeric protein refers to the polynucleotide capable encoding a recombinant fusion protein, e.g., a single polypeptide having the extracellular domains described herein (and, optionally a linker). For example, in various embodiments, the chimeric protein is translated as a single unit in a cell.

In some embodiments, an extracellular domain refers to a portion of a transmembrane protein which is capable of interacting with the extracellular environment. In various embodiments, an extracellular domain refers to a portion of a transmembrane protein which is sufficient to bind to a ligand or receptor and effective transmit a signal to a cell. In various embodiments, an extracellular domain is the entire amino acid sequence of a transmembrane protein which is external of a cell or the cell membrane. In various embodiments, an extracellular domain is the that portion of an amino acid sequence of a transmembrane protein which is external of a cell or the cell membrane and is needed for signal transduction and/or ligand binding as may be assayed using methods know in the art (e.g., in vitro ligand binding and/or cellular activation assays).

In some embodiments, an immune inhibitory signal refers to a signal that diminishes or eliminates an immune response. For example, in the context of oncology, such signals may diminish or eliminate antitumor immunity. Under normal physiological conditions, inhibitory signal are useful in the maintenance of self-tolerance (e.g., prevention of autoimmunity) and also to protect tissues from damage when the immune system is responding to pathogenic infection. For instance, without limitation, immune inhibitory signal may be identified by detecting an increase in cellular proliferation, cytokine production, cell killing activity or phagocytic activity when such an inhibitory signal is blocked. Specific examples such inhibitory signals include blockade of PD-1 of PD-L1/L2 using antibody mediated blockade or through competitive inhibition of PD-L1/L2 using PD-1 containing fusion proteins. When such an inhibitory signal is blocked through inhibition of PD-L1/L2, it leads to enhance tumor killing activity by T cells because they are no longer being inhibited by PD-L1 or PD-L2. In another example, and inhibitory signal may be provided by CD47 to macrophages expressing CD172a. Binding of CD47 to CD172a typically inhibits the ability of a macrophage to phagocytose a target cell, which can be restored through blockade of CD47 with blocking antibodies or through competitive inhibition of CD47 using CD172a containing fusion proteins.

In some embodiments, an immune stimulatory signal refers to a signal that enhances an immune response. For example, in the context of oncology, such signals may enhance antitumor immunity. For instance, without limitation, immune stimulatory signal may be identified by directly stimulating proliferation, cytokine production, killing activity or phagocytic activity of leukocytes. Specific examples include direct stimulation of TNF superfamily receptors such as OX40, 4-1BB or CD40 using either receptor agonist antibodies or using fusion proteins encoding the ligands for such receptors (OX40L, 4-1BBL, CD40L, respectively). Stimulation from any one of these receptors may directly stimulate the proliferation and cytokine production of individual T cell subsets. Another example includes direct stimulation of an immune inhibitory cell with through a receptor that inhibits the activity of such an immune suppressor cell. This would include, for example, stimulation of CD4+FoxP3+ regulatory T cells with a GITR agonist antibody or GITRL containing fusion protein, which would reduce the ability of those regulatory T cells to suppress the proliferation of conventional CD4+ or CD8+ T cells. In another example, this would include stimulation of CD40 on the surface of an antigen presenting cell using a CD40 agonist antibody or a fusion protein containing CD40L, causing activation of antigen presenting cells including enhanced ability of those cells to present antigen in the context of appropriate native costimulatory molecules, including those in the B7 or TNF superfamily.

Membrane proteins typically consist of an extracellular domain, one or a series of trans-membrane domains, and an intracellular domain. Without wishing to be bound by theory, the extracellular domain of a membrane protein is responsible for interacting with a soluble or membrane bound receptor or ligand. Without wishing to be bound by theory, the trans-membrane domain(s) are responsible for localizing a protein to the plasma membrane. Without wishing to be bound by theory, the intracellular domain of a membrane protein is responsible for coordinating interactions with cellular signaling molecules to coordinate intracellular responses with the extracellular environment (or visa-versa). There are two types of single-pass membrane proteins, those with an extracellular amino terminus and intracellular carboxy terminus (type I) and those with an extracellular carboxy terminus and intracellular amino terminus (type II). Both type I and type II membrane proteins can be either receptors or ligands. For type I membrane proteins, the amino terminus of the protein faces outside the cell, and therefore contains the functional domains that are responsible for interacting with other binding partners (either ligands or receptors) in the extracellular environment. For type II membrane proteins, the carboxy terminus of the protein faces outside the cell, and therefore contains the functional domains that are responsible for interacting with other binding partners (either ligands or receptors) in the extracellular environment. Thus, these two types of proteins have opposite orientations to each other.

Because the outward facing domains of type I and type II membrane proteins are opposite, it is possible to link the extracellular domains of a type I and type II membrane protein such that the ‘outward facing’ domains of the molecules are also in opposing orientation to each other. The resulting construct would therefore consist of the extracellular domain of a type I membrane protein on the ‘left’ side of the molecule, connected to the extracellular domain of a type II membrane protein on the ‘right’ side of the molecule using a linker sequence. This construct could be produced by cloning of these three fragments (the extracellular domain of a type I protein, followed by a linker sequence, followed by the extracellular domain of a type II protein) into a vector (plasmid, viral or other) wherein the amino terminus of the complete sequence corresponded to the ‘left’ side of the molecule containing the type I protein and the carboxy terminus of the complete sequence corresponded to the ‘right’ side of the molecule containing the type II protein. Accordingly, in various embodiments, the present chimeric proteins are engineered as such.

In some embodiments, the extracellular domain may be used to produce a soluble protein to competitively inhibit signaling by that receptor's ligand. In some embodiments, the extracellular domain may be used to provide artificial signaling.

In some embodiments, the extracellular domain of a type I transmembrane protein is an immune inhibitory signal. In some embodiments, the extracellular domain of a type II transmembrane protein is an immune stimulatory signal.

In some embodiments, the present isolated polynucleotides encoding chimeric proteins that comprise an extracellular domain of a type I transmembrane protein, or a functional fragment thereof. In some embodiments, the present isolated polynucleotides encoding chimeric proteins that comprise an extracellular domain of a type II transmembrane protein, or a functional fragment thereof. In some embodiments, the present isolated polynucleotides encode chimeric proteins that comprise an extracellular domain of a type I transmembrane protein, or a functional fragment thereof, and an extracellular domain of a type II transmembrane protein, or a functional fragment thereof.

The activation of regulatory T cells is critically influenced by costimulatory and coinhibitory signals. Two major families of costimulatory molecules include the B7 and the tumor necrosis factor (TNF) families. These molecules bind to receptors on T cells belonging to the CD28 or TNF receptor families, respectively. Many well-defined coinhibitors and their receptors belong to the B7 and CD28 families.

In various embodiments, the isolated polynucleotides encode a chimeric protein that comprises an immune inhibitory receptor extracellular domain and an immune stimulatory ligand extracellular domain which can, without limitation, deliver an immune stimulation to a T cell while masking a tumor cell's immune inhibitory signals. In various embodiments, the chimeric protein delivers a signal that has the net result of T cell activation.

In some embodiments, the isolated polynucleotides encode a chimeric protein that comprises an immune inhibitory signal which is an ECD of a receptor of an immune inhibitory signal and this acts on a tumor cell that bears a cognate ligand of the immune inhibitory signal. In some embodiments, the isolated polynucleotides encode a chimeric protein that comprises an immune stimulatory signal which is an ECD of a ligand of an immune stimulatory signal and this acts on a T cell that bears a cognate receptor of the immune stimulatory signal. In some embodiments, the chimeric protein comprises both (i) an immune inhibitory signal which is a receptor of an immune inhibitory signal and this acts on a tumor cell that bears a cognate ligand of the immune inhibitory signal and (ii) an immune stimulatory signal which is a ligand of an immune stimulatory signal and this acts on a T cell that bears a cognate receptor of the immune stimulatory signal.

In some embodiments, the isolated polynucleotides encode a chimeric protein that comprises an extracellular domain of one or more of the immune-modulating agents described in Mahoney, Nature Reviews Drug Discovery 2015:14; 561-585, the entire contents of which are hereby incorporated by reference.

In one aspect, the present disclosure relates to an isolated polynucleotide encoding a chimeric protein having a general structure of: N terminus—(a)-(b)-(c)—C terminus, wherein: (a) is a first domain comprising an extracellular domain of T cell immunoreceptor with Ig and ITIM domains (TIGIT), or a variant or a fragment thereof that is capable of binding a TIGIT ligand, (c) is a second domain comprising an extracellular domain of glucocorticoid-induced TNFR-related protein ligand (GITRL), or a variant or a fragment thereof that is capable of binding a GITRL receptor, or LIGHT (homologous to lymphotoxin, exhibits inducible expression and competes with HSV glycoprotein D for binding to herpesvirus entry mediator, a receptor expressed on T lymphocytes), or a variant or a fragment thereof that is capable of binding a LIGHT receptor, (b) is a linker adjoining the first and second domains, wherein the linker comprises at least one cysteine residue capable of forming a disulfide bond and/or comprises a hinge-CH2-CH3 Fc domain. In embodiments, the TIGIT ligand is selected from CD155/PVR, Nectin-2, Nectin-3 and Nectin-4. In embodiments, the GITRL receptor is glucocorticoid-induced TNFR-related protein (GITR). In embodiments, the LIGHT receptor is TNFRSF3/LTBR.

In embodiments, a heterologous chimeric protein of the present disclosure comprises, or the isolated polynucleotide encoding the heterologous chimeric protein encodes, the extracellular domain of human T cell immunoreceptor with Ig and ITIM domains (TIGIT), which comprises the following amino acid sequence:

(SEQ ID NO: 59)
MMTGTIETTGNISAEKGGSIILQCHLSSTTAQVTQVNWEQQDQLLAICN
ADLGWHISPSFKDRVAPGPGLGLTLQSLTVNDTGEYFCIYHTYPDGTYT
GRIFLEVLESSVAEHGARFQIP.

In embodiments, a heterologous chimeric protein used in methods of the present disclosure comprises a variant of the extracellular domain of TIGIT. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 59.

In embodiments, the first domain of a heterologous chimeric protein comprises an amino acid sequence that is at least 95% identical to the amino acid sequence of SEQ ID NO: 59.

In embodiments, a heterologous chimeric protein comprises substantially the entire extracellular domain of TIGIT.

One of ordinary skill may select variants of the known amino acid sequence of TIGIT by consulting the literature, e.g., Stengel et al., Structure of TIGIT immunoreceptor bound to poliovirus receptor reveals a cell-cell adhesion and signaling mechanism that requires cis-trans receptor clustering, Proc Natl Acad Sci USA 109: 5399-5404 (2012); Deuss et al., Recognition of nectin-2 by the natural killer cell receptor T cell immunoglobulin and ITIM domain (TIGIT), J Biol Chem 292: 11413-11422 (2017); Varadi et al., AlphaFold Protein Structure Database: massively expanding the structural coverage of protein-sequence space with high-accuracy models, Nucleic Acids Research, 50(D1); D439-D444 (2022); and Uniprot structure 3RQ3; each of which is incorporated by reference in its entirety.

In embodiments, a heterologous chimeric protein of the present disclosure comprises, or the isolated polynucleotide encoding the heterologous chimeric protein encodes, the extracellular domain of human glucocorticoid-induced TNFR-related protein ligand (GITRL), which comprises the following amino acid sequence:

(SEQ ID NO: 60)
ETAKEPCMAKFGPLPSKWQMASSEPPCVNKVSDWKLEILQNGLYLIYGQ
VAPNANYNDVAPFEVRLYKNKDMIQTLTNKSKIQNVGGTYELHVGDTID
LIFNSEHQVLKNNTYWGIILLANPQFIS.

In embodiments, a heterologous chimeric protein used in methods of the present disclosure comprises a variant of the extracellular domain of GITRL As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 60.

In embodiments, the first domain of a heterologous chimeric protein comprises an amino acid sequence that is at least 95% identical to the amino acid sequence of SEQ ID NO: 60.

In embodiments, a heterologous chimeric protein comprises substantially the entire extracellular domain of GITRL.

One of ordinary skill may select variants of the known amino acid sequence of GITRL by consulting the literature, e.g., Chattopadhyay et al., Assembly and structural properties of glucocorticoid-induced TNF receptor ligand: Implications for function, Proc Natl Acad Sci USA 104: 19452-19457 (2007); Wang et al., Structures of mouse and human GITR-GITRL complexes reveal unique TNF superfamily interactions, Nat Commun 12: 1378-1378 (2021); Chattopadhyay et al., “Evolution of GITRL immune function: Murine GITRL exhibits unique structural and biochemical properties within the TNF superfamily.” Proc Natl Acad Sci USA, 105(2): 635-640 (2008); and Zhou, et al. “Structural basis for ligand-mediated mouse GITR activation Structural basis for ligand-mediated mouse GITR activation.” Proc Natl Acad Sci USA, 105 (2) 641-645; (2008); and Uniprot structures 3B93, 3B94; each of which is incorporated by reference in its entirety.

In embodiments, a heterologous chimeric protein of the present disclosure comprises, or the isolated polynucleotide encoding the heterologous chimeric protein encodes, the extracellular domain of human LIGHT, which comprises the following amino acid sequence:

LQLHWRLGEMVTRLPDGPAGSWEQLIQERRSHEVNPAAHLTGANSSLTGSGGPLLWETQLGLAFLRGLS YHDGALVVTKAGYYYIYSKVQLGGVGCPLGLASTITHGLYKRTPRYPEELELLVSQQSPCGRATSSSRVW WDSSFLGGVVHLEAGEKVVVRVLDERLVRLRDGTRSYFGAFMV (SEQ ID NO: 61).

In embodiments, a heterologous chimeric protein used in methods of the present disclosure comprises a variant of the extracellular domain of LIGHT As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 61.

In embodiments, the first domain of a heterologous chimeric protein comprises an amino acid sequence that is at least 95% identical to the amino acid sequence of SEQ ID NO: 61.

In embodiments, a heterologous chimeric protein comprises substantially the entire extracellular domain of LIGHT.

One of ordinary skill may select variants of the known amino acid sequence of LIGHT by consulting the literature, e.g., Liu et al., Mechanistic basis for functional promiscuity in the TNF and TNF receptor superfamilies: structure of the LIGHT:DcR3 assembly, Structure 22: 1252-1262 (2014); Liu et al., HVEM structures and mutants reveal distinct functions of binding to LIGHT and BTLA/CD160, J Exp Med 218 (2021), each of which is incorporated by reference in its entirety.

In one aspect, the present disclosure relates to an isolated polynucleotide encoding a chimeric protein having a general structure of: N terminus—(a)-(b)-(c)—C terminus, wherein: (a) is a first domain comprising an extracellular domain of tumor necrosis factor (TNF) receptor 2 (TNFR2), or a variant or a fragment thereof that is capable of binding a TNFR2 ligand, (c) is a second domain comprising an extracellular domain selected from CLEC7a, or a variant or a fragment thereof that capable of binding a CLEC7a ligand, DC-SIGN(CD209), or a variant or a fragment thereof that capable of binding a DC-SIGN(CD209) ligand, DECTIN2(CLEC6A), or a variant or a fragment thereof that capable of binding a DECTIN2(CLEC6A) ligand, Langerin(CD207,CLC4K), or a variant or a fragment thereof that capable of binding a Langerin(CD207,CLC4K) ligand, CD69, or a variant or a fragment thereof that capable of binding a CD69 ligand, and TGF-beta, or a variant or a fragment thereof that capable of binding a TGF-beta receptor, (b) is a linker adjoining the first and second domains, wherein the linker comprises at least one cysteine residue capable of forming a disulfide bond and/or comprises a hinge-CH2-CH3 Fc domain. In embodiments, the TNFR2 ligand is TNFα. In embodiments, the CLEC7a ligand is a beta-1,3-linked and/or beta-1,6-linked glucan. In embodiments, the DC-SIGN(CD209) ligand is a Intercellular Adhesion Molecule 2 (ICAM2) and/or Intercellular Adhesion Molecule 3 (ICAM3). In embodiments, the DECTIN2(CLEC6A) ligand is an alpha-mannan. In embodiments, the Langerin(CD207,CLC4K) ligand is a sulfated glycan, a mannosylated glycan, a keratan sulfate (KS) and/or a beta-glucan. In embodiments, the CD69 ligand is Galectin-1 (Gal-1) or the S100A8/S100A9 complex. In embodiments, the TGF-beta receptor is TGFBR1 and TGFBR2. In embodiments, the TGF-beta receptor is TGFBR1 and TGFBR2.

In embodiments, the extracellular domain of TNFR2, or a fragment thereof inhibits TNFα by competing with the cellular receptor species for TNF binding by sequestering. In embodiments, first domain inhibits TNFα by oligomerizing with cellular TNF receptor species, forming inactive complexes, and thereby inhibiting the function of the cellular TNF receptor species

In embodiments, a heterologous chimeric protein of the present disclosure comprises, or the isolated polynucleotide encoding the heterologous chimeric protein encodes, the extracellular domain of human TNFR2, which comprises the following amino acid sequence:

(SEQ ID NO: 62)
LPAQVAFTPYAPEPGSTCRLREYYDQTAQMCCSKCSPGQHAKVFCTKTS
DTVCDSCEDSTYTQLWNWVPECLSCGSRCSSDQVETQACTREQNRICTC
RPGWYCALSKQEGCRLCAPLRKCRPGFGVARPGTETSDWVCKPCAPGTF
SNTTSSTDICRPHQICNVVAIPGNASMDAVCTSTSPTRSMAPGAVHLPQ
PVSTRSQHTQPTPEPSTAPSTSFLLPMGPSPPAEGSTGD.

In embodiments, a heterologous chimeric protein used in methods of the present disclosure comprises a variant of the extracellular domain of TNFR2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 62.

In embodiments, the first domain of a heterologous chimeric protein comprises an amino acid sequence that is at least 95% identical to the amino acid sequence of SEQ ID NO: 62.

In embodiments, a heterologous chimeric protein comprises substantially the entire extracellular domain of TNFR2.

One of ordinary skill may select variants of the known amino acid sequence of TNFR2 by consulting the literature, e.g., Kohno et al., “A second tumor necrosis factor receptor gene product can shed a naturally occurring tumor necrosis factor inhibitor.” Proc. Natl. Acad. Sci. U.S.A. 87 (21), 8331-8335 (1990); Smith et al., “A receptor for tumor necrosis factor defines an unusual family of cellular and viral proteins.” Science 248 (4958), 1019-1023 (1990); Loetscher et al., “Purification and partial amino acid sequence analysis of two distinct tumor necrosis factor receptors from HL60 cells.” J. Biol. Chem. 265 (33), 20131-20138 (1990); Dembic, et al., “Two human TNF receptors have similar extracellular, but distinct intracellular, domain sequences.” Cytokine 2 (4), 231-237 (1990); Pennica et al., “Biochemical properties of the 75-kDa tumor necrosis factor receptor. Characterization of ligand binding, internalization, and receptor phosphorylation.” J. Biol. Chem. 267 (29), 21172-21178 (1992); and Park et al., “Structural basis for self-association and receptor recognition of human TRAF2.” Nature 398 (6727), 533-538 (1999); Mukai et al., “Solution of the structure of the TNF-TNFR2 complex.” Sci Signal. 3(148):ra83 (2010); TNF-TNFR2 structure PDB ID: 3ALQ, each of which is incorporated by reference in its entirety.

In embodiments, a heterologous chimeric protein of the present disclosure comprises, or the isolated polynucleotide encoding the heterologous chimeric protein encodes, the extracellular domain of human CLEC7A, which comprises the following amino acid sequence:

(SEQ ID NO: 63)
TMAIWRSNSGSNTLENGYFLSRNKENHSQPTQSSLEDSVTPTKAVKTTG
VLSSPCPPNWIIYEKSCYLFSMSLNSWDGSKRQCWQLGSNLLKIDSSNE
LGFIVKQVSSQPDNSFWIGLSRPQTEVPWLWEDGSTFSSNLFQIRTTAT
QENPSPNCVWIHVSVIYDQLCSVPSYSICEKKFSM.

In embodiments, a heterologous chimeric protein used in methods of the present disclosure comprises a variant of the extracellular domain of CLEC7A. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 63.

In embodiments, the first domain of a heterologous chimeric protein comprises an amino acid sequence that is at least 95% identical to the amino acid sequence of SEQ ID NO: 63.

In embodiments, a heterologous chimeric protein comprises substantially the entire extracellular domain of CLEC7A.

In embodiments, the second domain comprises the C-type lectin binding domain (CLD) of Clec7a, which has the following sequence:

(SEQ ID NO: 64)
SSPCPPNWIIYEKSCYLFSMSLNSWDGSKRQCWQLGSNLLKIDSSNELG
FIVKQVSSQPDNSFWIGLSRPQTEVPWLWEDGSTFSSNLFQIRTTATQE
NPSPNCVWIHVSVIYDQLCSVPSYSICEKKFSM

In embodiments, the chimeric protein comprises a variant of the C-type lectin binding domain (CLD) of Clec7a. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 64.

In embodiments, the portion of Clec7a comprises the extracellular domain of Clec7a, or a fragment thereof. In embodiments, the portion of Clec7a comprises an amino acid sequence that is at least about 90%, or at least about 95%, or at least about 97%, or at least about 98%, or at least about 99% identical to the amino acid sequence of SEQ ID NO: 63 or SEQ ID NO: 64. In embodiments, the first domain of a chimeric protein comprises an amino acid sequence that is at least 95% identical to the amino acid sequence of SEQ ID NO: 63 or SEQ ID NO: 64.

One of ordinary skill may select variants of the known amino acid sequence of Clec7a by consulting the literature, e.g., Brown et al., Structure of the Fungal Beta-Glucan-Binding Immune Receptor Dectin-1: Implications for Function. Protein Sci 16: 1042-1052 (2007); TNF-TNFR2 structure PDB ID: 2BPE; Alphafold structure (Jumper et al., Highly accurate protein structure prediction with AlphaFold. Nature 596: 583-589 (2021)); Legentil et al., Molecular Interactions of β-(1→3)-Glucans with Their Receptors, Molecules 20(6):9745-66 (2015), each of which is incorporated by reference in its entirety.

In embodiments, a heterologous chimeric protein of the present disclosure comprises, or the isolated polynucleotide encoding the heterologous chimeric protein encodes, a portion of DC-SIGN (also known as C-type lectin domain family 4 member L or CD209). In embodiments, the portion of DC-SIGN comprises the extracellular domain of DC-SIGN, or a fragment thereof capable of binding an Intercellular Adhesion Molecule 2 (ICAM2) and/or Intercellular Adhesion Molecule 3 (ICAM3).

DC-SIGN is pathogen-recognition receptor expressed on the surface of immature dendritic cells (DCs) and involved in initiation of primary immune response. It is thought to mediate the endocytosis of pathogens which are subsequently degraded in lysosomal compartments.

In embodiments, the second domain comprises the extracellular domain of DC-SIGN, which has the following sequence:

(SEQ ID NO: 65)
QVSKVPSSISQEQSRQDAIYQNLTQLKAAVGELSEKSKLQEIYQELTQL
KAAVGELPEKSKLQEIYQELTRLKAAVGELPEKSKLQEIYQELTWLKAA
VGELPEKSKMQEIYQELTRLKAAVGELPEKSKQQEIYQELTRLKAAVGE
LPEKSKQQEIYQELTRLKAAVGELPEKSKQQEIYQELTQLKAAVERLCH
PCPWEWTFFQGNCYFMSNSQRNWHDSITACKEVGAQLVVIKSAEEQNFL
QLQSSRSNRFTWMGLSDLNQEGTWQWVDGSPLLPSFKQYWNRGEPNNVG
EEDCAEFSGNGWNDDKCNLAKFWICKKSAASCSRDEEQFLSPAPATPNP
PPA.

In embodiments, the chimeric protein comprises a variant of the extracellular domain (ECD) of DC-SIGN. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 65.

In embodiments, a heterologous chimeric protein of the present disclosure comprises, or the isolated polynucleotide encoding the heterologous chimeric protein encodes, the C-type lectin binding domain (CLD) of DC-SIGN, which has the following sequence:

(SEQ ID NO: 66)
HPCPWEWTFFQGNCYFMSNSQRNWHDSITACKEVGAQLVVIKSAEEQNF
LQLQSSRSNRFTWMGLSDLNQEGTWQWVDGSPLLPSFKQYWNRGEPNNV
GEEDCAEFSGNGWNDDKCNLAKFWICK

In embodiments, the chimeric protein comprises a variant of the C-type lectin binding domain (CLD) of DC-SIGN. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 66.

In embodiments, the portion of DC-SIGN comprises the extracellular domain of DC-SIGN, or a fragment thereof. In embodiments, the portion of DC-SIGN comprises an amino acid sequence that is at least about 90%, or at least about 95%, or at least about 97%, or at least about 98%, or at least about 99% identical to the amino acid sequence of SEQ ID NO: 65 or SEQ ID NO: 66. In embodiments, the first domain of a chimeric protein comprises an amino acid sequence that is at least 95% identical to the amino acid sequence of SEQ ID NO: 65 or SEQ ID NO: 66.

One of ordinary skill may select variants of the known amino acid sequence of DC-SIGN by consulting the literature and structural information, e.g., Feinberg et al., Structural basis for selective recognition of oligosaccharides by DC-SIGN and DC-SIGNR, Science 294: 2163-6 (2001); Guo et al., Structural basis for distinct ligand-binding and targeting properties of the receptors DC-SIGN and DC-SIGNR, Nat. Struct. Mol. Biol. 11: 591-8 (2004); Pokidysheva et al., Cryo-EM reconstruction of dengue virus in complex with the carbohydrate recognition domain of DC-SIGN, Cell 124: 485-93 (2006); Feinberg et al., Multiple modes of binding enhance the affinity of DC-SIGN for high mannose N-linked glycans found on viral glycoproteins. J. Biol. Chem. 282 4202-9 (2007); Thepaut et al., Structure of a glycomimetic ligand in the carbohydrate recognition domain of C-type lectin DC-SIGN. Structural requirements for selectivity and ligand design. J. Am. Chem. Soc. 135 2518-29 (2013); Medve et al., Enhancing potency and selectivity of a DC-SIGN glycomimetic ligand by fragment-based design: structural basis. Chemistry 25(64):14659-14668 (2019); Sutkeviciute et al., Unique DC-SIGN Clustering Activity of a Small Glycomimetic: A Lesson for Ligand Design. ACS Chem. B. 9(6):1377-1385 (2014); Porkolab et al., Rational-Differential Design of Highly Specific Glycomimetic Ligands: Targeting DC-SIGN and Excluding Langerin Recognition. ACS Chem. Biol. 13(3): 600-608 (2018); Alphafold structure (Jumper et al., Highly accurate protein structure prediction with AlphaFold. Nature 596: 583-589 (2021)), each of which is incorporated by reference in its entirety.

In embodiments, a heterologous chimeric protein of the present disclosure comprises, or the isolated polynucleotide encoding the heterologous chimeric protein encodes, a portion of Dectin-2 (also known as C-type lectin domain family 6 member A or C-type lectin superfamily member 10). In embodiments, the portion of Dectin-2 comprises the extracellular domain of Dectin-2, or a fragment thereof capable of binding an alpha-mannan.

Dectin-2 is a calcium-dependent lectin that acts as a pattern recognition receptor (PRR) of the innate immune system: specifically recognizes and binds alpha-mannans on C. albicans hypheas. In embodiments, the portion of Dectin-2 comprises the extracellular domain of Dectin-2, or a fragment thereof capable of recognizing allergens from house dust mite and fungi in a mannose-dependent manner, and/or soluble elements from the eggs of Schistosoma mansoni altering adaptive immune responses.

In embodiments, the second domain comprises the extracellular domain of Dectin-2, which has the following sequence:

(SEQ ID NO: 67)
TYHFTYGETGKRLSELHSYHSSLTCFSEGTKVPAWGCCPASWKSFGSSC
YFISSEEKVWSKSEQNCVEMGAHLVVFNTEAEQNFIVQQLNESFSYFLG
LSDPQGNNNWQWIDKTPYEKNVRFWHLGEPNHSAEQCASIVFWKPTGWG
WNDVICETRRNSICEMNKIYL.

In embodiments, the chimeric protein comprises a variant of the extracellular domain of Dectin-2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 67.

In embodiments, a heterologous chimeric protein of the present disclosure comprises, or the isolated polynucleotide encoding the heterologous chimeric protein encodes, the C-type lectin binding domain (CLD) of Dectin-2, which has the following sequence:

(SEQ ID NO: 68)
FGSSCYFISSEEKVWSKSEQNCVEMGAHLVVFNTEAEQNFIVQQLNESF
SYFLGLSDPQGNNNWQWIDKTPYEKNVRFWHLGEPNHSAEQCASIVFWK
PTGWGWNDVICETRRNSICE

In embodiments, the chimeric protein comprises a variant of the C-type lectin binding domain (CLD) of Dectin-2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 68.

In embodiments, the portion of Dectin-2 comprises the extracellular domain of Dectin-2, or a fragment thereof. In embodiments, the portion of Dectin-2 comprises an amino acid sequence that is at least about 90%, or at least about 95%, or at least about 97%, or at least about 98%, or at least about 99% identical to the amino acid sequence of SEQ ID NO: 67 or SEQ ID NO: 68. In embodiments, the first domain of a chimeric protein comprises an amino acid sequence that is at least 95% identical to the amino acid sequence of SEQ ID NO: 67 or SEQ ID NO: 68.

One of ordinary skill may select variants of the known amino acid sequence of Dectin-2 by consulting the literature and structural information, e.g., Feinberg et al., Mechanism of pathogen recognition by human dectin-2. J. Biol. Chem. 292(32):13402-13414 (2017); Decout et al., Deciphering the molecular basis of mycobacteria and lipoglycan recognition by the C-type lectin Dectin-2, Scientific Reports 8: 16840 (2018); McGreal et al., The carbohydrate-recognition domain of Dectin-2 is a C-type lectin with specificity for high mannose, Glycobiology, 16(5): 422-430 (2006); Alphafold structure (Jumper et al., Highly accurate protein structure prediction with AlphaFold. Nature 596: 583-589 (2021)), each of which is incorporated by reference in its entirety.

In embodiments, a heterologous chimeric protein of the present disclosure comprises, or the isolated polynucleotide encoding the heterologous chimeric protein encodes, a portion of langerin (also known as C-type lectin domain family 4 member K or CD207). In embodiments, the portion of langerin comprises the extracellular domain of langerin, or a fragment thereof capable of binding a sulfated glycan, a mannosylated glycan, a keratan sulfate (KS) and/or a beta-glucan.

Langerin is a calcium-dependent lectin displaying mannose-binding specificity. It facilitates uptake of antigens and is involved in the routing and/or processing of antigen for presentation to T cells. Langerin is a major receptor on primary Langerhans cells for Candida species, Saccharomyces species, and Malassezia furfur. It binds to high-mannose structures present on the envelope glycoprotein of HIV virus, which is followed by subsequent targeting of the virus to the Birbeck granules leading to its rapid degradation.

In embodiments, the second domain comprises the extracellular domain (ECD) of langerin, which has the following sequence:

(SEQ ID NO: 69)
PRFMGTISDVKTNVQLLKGRVDNISTLDSEIKKNSDGMEAAGVQIQMVN
ESLGYVRSQFLKLKTSVEKANAQIQILTRSWEEVSTLNAQIPELKSDLE
KASALNTKIRALQGSLENMSKLLKRQNDILQVVSQGWKYFKGNFYYFSL
IPKTWYSAEQFCVSRNSHLTSVTSESEQEFLYKTAGGLIYWIGLTKAGM
EGDWSWVDDTPFNKVQSVRFWIPGEPNNAGNNEHCGNIKAPSLQAWNDA
PCDKTFLFICKRPYVPSEP.

In embodiments, the chimeric protein comprises a variant of the extracellular domain of langerin. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 69.

In embodiments, the second domain comprises the C-type lectin binding domain (CLD) of langerin, which has the following sequence:

(SEQ ID NO: 70)
QVVSQGWKYFKGNFYYFSLIPKTWYSAEQFCVSRNSHLTSVTSESEQEF
LYKTAGGLIYWIGLTKAGMEGDWSWVDDTPFNKVQSVRFWIPGEPNNAG
NNEHCGNIKAPSLQAWNDAPCDKTFLFICKRPYVPSEP

In embodiments, the chimeric protein comprises a variant of the C-type lectin binding domain (CLD) of langerin. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 70.

In embodiments, the portion of langerin comprises the extracellular domain of langerin, or a fragment thereof. In embodiments, the portion of langerin comprises an amino acid sequence that is at least about 90%, or at least about 95%, or at least about 97%, or at least about 98%, or at least about 99% identical to the amino acid sequence of SEQ ID NO: 69 or SEQ ID NO: 70. In embodiments, the first domain of a chimeric protein comprises an amino acid sequence that is at least 95% identical to the amino acid sequence of SEQ ID NO: 69 or SEQ ID NO: 70.

One of ordinary skill may select variants of the known amino acid sequence of langerin by consulting the literature and structural information, e.g., Nurisso et al., Structural studies of langerin and Birbeck granule: a macromolecular organization model, Biochemistry 48: 2684-98 (2009); Feinberg et al., Trimeric structure of langerin. J. Biol. Chem. 285: 13285-93 (2010); Feinberg et al., Structural basis for langerin recognition of diverse pathogen and mammalian glycans through a single binding site. J. Mol. Biol. 405: 1027-39 (2011); Chatwell et al., The carbohydrate recognition domain of langerin reveals high structural similarity with the one of DC-SIGN but an additional, calcium-independent sugar-binding site, Mol. Immunol. 45: 1981-94 (2008); Chabrol et al., Alteration of the langerin oligomerization state affects birbeck granule formation, Biophys. J. 108: 666-77 (2015); Feinberg et al., Common polymorphisms in human langerin change specificity for glycan ligands. J. Biol. Chem. 288(52):36762-36771 (2013); Porkolab et al., Rational-Differential Design of Highly Specific Glycomimetic Ligands: Targeting DC-SIGN and Excluding Langerin Recognition. ACS Chem. Biol. 13(3): 690-608 (2018); Alphafold structure (Jumper et al., Highly accurate protein structure prediction with AlphaFold. Nature 596: 583-589 (2021)), each of which is incorporated by reference in its entirety.

In embodiments, a heterologous chimeric protein of the present disclosure comprises, or the isolated polynucleotide encoding the heterologous chimeric protein encodes, the extracellular domain of human CD69, which comprises the following amino acid sequence:

(SEQ ID NO: 71)
LPAQVAFTPYAPEPGSTCRLREYYDQTAQMCCSKCSPGQHAKVFCTKTS
DTVCDSCEDSTYTQLWNWVPECLSCGSRCSSDQVETQACTREQNRICTC
RPGWYCALSKQEGCRLCAPLRKCRPGFGVARPGTETSDWCKPCAPGTFS
NTTSSTDICRPHQICNVVAIPGNASMDAVCTSTSPTRSMAPGAVHLPQP
VSTRSQHTQPTPEPSTAPSTSFLLPMGPSPPAEGSTGD.

In embodiments, a heterologous chimeric protein used in methods of the present disclosure comprises a variant of the extracellular domain of CD69. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 71.

In embodiments, the first domain of a heterologous chimeric protein comprises an amino acid sequence that is at least 95% identical to the amino acid sequence of SEQ ID NO: 71.

In embodiments, a heterologous chimeric protein comprises substantially the entire extracellular domain of CD69.

One of ordinary skill may select variants of the known amino acid sequence of CD69 by consulting the literature, e.g., Llera et al., Crystal Structure of the C-Type Lectin-Like Domain from the Human Hematopoietic Cell Receptor CD69, J Biol Chem 276(10):7312-7319 (2001); Natarajan et al., Crystal structure of human CD69: a C-type lectin-like activation marker of hematopoietic cells, Biochemistry 39: 14779-14786 (2000); Vanek et al., Soluble recombinant CD69 receptors optimized to have an exceptional physical and chemical stability display prolonged circulation and remain intact in the blood of mice, FEBS J 275: 5589-5606 (2008); Kolenko et al., The high-resolution structure of the extracellular domain of human CD69 using a novel polymer, Acta Crystallogr Sect F Struct Biol Cryst Commun 65: 1258-1260 (2009), each of which is incorporated by reference in its entirety.

In embodiments, a heterologous chimeric protein of the present disclosure comprises, or the isolated polynucleotide encoding the heterologous chimeric protein encodes, the TGF-beta, which comprises the following amino acid sequence:

(SEQ ID NO: 72)
ALDTNYCFSSTEKNCCVRQLYIDFRKDLGWKWIHEPKGYHANFCLGPCP
YIWSLDTQYSKVLALYNQHNPGASAAPCCVPQALEPLPIVYYVGRKPKV
EQLSNMIVRSCKCS.

In embodiments, a chimeric protein comprises a variant of the extracellular domain of TGF-beta. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 72.

One of ordinary skill may select variants of the known amino acid sequence of TGF-beta by consulting the literature, e.g., Hinck et al., (1996) Transforming growth factor beta 1: three-dimensional structure in solution and comparison with the X-ray structure of transforming growth factor beta 2, Biochemistry 35: 8517-8534; Radaev et al., (2010) Ternary complex of transforming growth factor-beta1 reveals isoform-specific ligand recognition and receptor recruitment in the superfamily, J Biol Chem 285: 14806-14814; Dong et al., (2017) Force interacts with macromolecular structure in activation of TGF-beta, Nature 542: 55-59, each of which is incorporated by reference in its entirety.

In one aspect, the present disclosure relates to an isolated polynucleotide encoding a chimeric protein having a general structure of: N terminus—(a)-(b)-(c)—C terminus, wherein: (a) is a first domain comprising an extracellular domain of programmed death-1 (PD-1), or a variant or a fragment thereof that capable of binding a PD-1 ligand, (c) is a second domain comprising an extracellular domain of OX40 ligand (OX40L), or a variant or a fragment thereof that capable of binding an OX40L receptor, or 4-1BB Ligand (4-1BBL), or a variant or a fragment thereof that capable of binding a 4-1BBL receptor, (b) is a linker adjoining the first and second domains, wherein the linker comprises at least one cysteine residue capable of forming a disulfide bond and/or comprises a hinge-CH2-CH3 Fc domain. In embodiments, the PD-1 ligand is PD-L1 or PD-L2. In embodiments, the OX40L receptor is OX40. In embodiments, the 4-1BBL receptor is 4-1BB.

In embodiments, a heterologous chimeric protein of the present disclosure comprises, or the isolated polynucleotide encoding the heterologous chimeric protein encodes, the extracellular domain of human PD-1, which comprises the following amino acid sequence:

(SEQ ID NO: 73)
LDSPDRPWNPPTFSPALLVVTEGDNATFTCSFSNTSESFVLNWYRMSPS
NQTDKLAAFPEDRSQPGQDCRFRVTQLPNGRDFHMSVVRARRNDSGTYL
CGAISLAPKAQIKESLRAELRVTERRAEVPTAHPSPSPRPAGQFQ.

In embodiments, a heterologous chimeric protein used in methods of the present disclosure comprises a variant of the extracellular domain of PD-1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 73.

In embodiments, the first domain of a heterologous chimeric protein comprises an amino acid sequence that is at least 95% identical to the amino acid sequence of SEQ ID NO: 73.

In embodiments, a heterologous chimeric protein comprises substantially the entire extracellular domain of PD-1.

One of ordinary skill may select variants of the known amino acid sequence of PD-1 by consulting the literature, e.g., Zhang et al“Structural and Functional Analysis of the Costimulatory Receptor Programmed Death-1” Immunity. 2004 March; 20(3):337-47; Lin et al “The PD-1/PD-L1 complex resembles the antigen-binding Fv domains of antibodies and T cell receptors”, Proc Natl Acad Sci USA. 2008 Feb. 26; 105(8):3011-6; Zak et al“Structure of the Complex of Human Programmed Death 1, PD-1, and Its Ligand PD-L1”, Structure. 2015 Dec. 1; 23(12):2341-2348; and Cheng et al“Structure and Interactions of the Human Programmed Cell Death 1 Receptor”, J Biol Chem. 2013 Apr. 26; 288(17):11771-85, each of which is incorporated by reference in its entirety.

In embodiments, a heterologous chimeric protein of the present disclosure comprises, or the isolated polynucleotide encoding the heterologous chimeric protein encodes, the extracellular domain of human OX40L, which comprises the following amino acid sequence:

(SEQ ID NO: 74)
QVSHRYPRIQSIKVQFTEYKKEKGFILTSQKEDEIMKVQNNSVIINCDG
FYLISLKGYFSQEVNISLHYQKDEEPLFQLKKVRSVNSLMVASLTYKDK
VYLNVTTDNTSLDDFHVNGGELILIHQNPGEFCVL.

In embodiments, a chimeric protein comprises a variant of the extracellular domain of OX40L. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 74.

In embodiments, the second domain of a chimeric protein comprises an amino acid sequence that is at least 95% identical to the amino acid sequence of SEQ ID NO: 74.

One of ordinary skill may select variants of the known amino acid sequence of OX40L by consulting the literature, e.g., Croft, et al., “The Significance of OX40 and OX40L to T cell Biology and Immune Disease,” Immunol Rev., 229(1), PP. 173-191, 2009 and Baum, et al., “Molecular characterization of murine and human OX40/OX40 ligand systems: identification of a human OX40 ligand as the HTL V-1-regulated protein gp34,” The EMBO Journal, Vol. 13, No. 77, PP. 3992-4001, 1994; and Compaan and Hymowitz, The Crystal Structure of the Costimulatory OX40-OX40L Complex. Structure 14: 1321-1330 (2006), each of which is incorporated by reference in its entirety.

In embodiments, a heterologous chimeric protein of the present disclosure comprises, or the isolated polynucleotide encoding the heterologous chimeric protein encodes, the extracellular domain of human 4-1BBL, which comprises the following amino acid sequence:

(SEQ ID NO: 75)
ACPWAVSGARASPGSAASPRLREGPELSPDDPAGLLDLRQGMFAQLVAQ
NVLLIDGPLSWYSDPGLAGVSLTGGLSYKEDTKELVVAKAGVYYVFFQL
ELRRVVAGEGSGSVSLALHLQPLRSAAGAAALALTVDLPPASSEARNSA
FGFQGRLLHLSAGQRLGVHLHTEARARHAWQLTQGATVLGLFRVTPEIP
AGLPSPRSE.

In embodiments, a heterologous chimeric protein used in methods of the present disclosure comprises a variant of the extracellular domain of 4-1BBL. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 75.

In embodiments, the first domain of a heterologous chimeric protein comprises an amino acid sequence that is at least 95% identical to the amino acid sequence of SEQ ID NO: 75.

In embodiments, a heterologous chimeric protein comprises substantially the entire extracellular domain of 4-1BBL.

One of ordinary skill may select variants of the known amino acid sequence of 4-1BBL by consulting the literature, e.g., Won et al., “The structure of the trimer of human 4-1BB ligand is unique among members of the tumor necrosis factor superfamily.” J. Biol. Chem. 285: 9202-9210 (2010); Gilbreth et al., “Crystal structure of the human 4-1BB/4-1BBL complex.” J Biol Chem 293: 9880-9891 (2018); and Bitra et al., “Crystal structures of the human 4-1BB receptor bound to its ligand 4-1BBL reveal covalent receptor dimerization as a potential signaling amplifier.” J Biol Chem 293: 9958-9969 (2018); Li et al., (2018) Limited Cross-Linking of 4-1BB by 4-1BB Ligand and the Agonist Monoclonal Antibody Utomilumab, Cell Rep 25: 909-920.e4 and Uniprot structures 3B93, 3B94; each of which is incorporated by reference in its entirety.

In one aspect, the present disclosure relates to an isolated polynucleotide encoding a chimeric protein having a general structure of: N terminus—(a)-(b)-(c)—C terminus, wherein: (a) is a first domain comprising an extracellular domain of T-cell immunoglobulin mucin receptor 3 (TIM3), or a variant or a fragment thereof that capable of binding a TIM3 ligand, (c) is a second domain comprising an extracellular domain of CD40 ligand (CD40L), or a variant or a fragment thereof that capable of binding a CD40L receptor, (b) is a linker adjoining the first and second domains, wherein the linker comprises at least one cysteine residue capable of forming a disulfide bond and/or comprises a hinge-CH2-CH3 Fc domain. In embodiments, the TIM3 ligand is selected from galectin 9, phosphatidylserine (PtdSer), CEACAM1 and high mobility group protein B1 (HMGB1). In embodiments, the CD40L receptor is CD40.

In embodiments, a heterologous chimeric protein of the present disclosure comprises, or the isolated polynucleotide encoding the heterologous chimeric protein encodes, the extracellular domain of human T-cell immunoglobulin mucin receptor 3 (TIM3), which comprises the following amino acid sequence:

(SEQ ID NO: 76)
SEVEYRAEVGQNAYLPCFYTPAAPGNLVPVCWGKGACPVFECGNVVLRT
DERDVNYWTSRYWLNGDFRKGDVSLTIENVTLADSGIYCCRIQIPGIMN
DEKFNLKLVIKPAKVTPAPTRQRDFTAAFPRMLTTRGHGPAETQTLGSL
PDINLTQISTLANELRDSRLANDLRDSGATIRIG.

In embodiments, a heterologous chimeric protein comprises substantially the entire extracellular domain of TIM3.

In embodiments, a chimeric protein comprises a variant of the extracellular domain of TIM-3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 76.

In embodiments, the first domain of a chimeric protein comprises an amino acid sequence that is at least 95% identical to the amino acid sequence of SEQ ID NO: 76.

One of ordinary skill may select variants of the known amino acid sequence of TIM-3 by consulting the literature, e.g., Cao, et al., “T Cell Immunoglobulin Mucin-3 Crystal Structure Reveals a Galectin-9-Independent Ligand-Binding Surface,” Immunity 26, pp. 311-321, 2007 and Freeman, et al., “TIM genes: a family of cell surface phosphatidylserine receptors that regulate innate and adaptive immunity,” Immunol Rev., 235(1), pp. 172-189, 2010; Karpusas et al., 2 A crystal structure of an extracellular fragment of human CD40 ligand, Structure 3: 1031-1039 (1995); Karpusas et al., Structure of CD40 ligand in complex with the Fab fragment of a neutralizing humanized antibody, Structure 9: 321-329 (2001); Silvian et al., Small Molecule Inhibition of the TNF Family Cytokine CD40 Ligand through a Subunit Fracture Mechanism, ACS Chem Biol 6: 636-647 (2011); An et al., Crystallographic and mutational analysis of the CD40-CD154 complex and its implications for receptor activation, J Biol Chem 286: 11226-11235 (2011), each of which is incorporated by reference in its entirety.

In embodiments, a heterologous chimeric protein of the present disclosure comprises, or the isolated polynucleotide encoding the heterologous chimeric protein encodes, the extracellular domain of human CD40L, which comprises the following amino acid sequence:

(SEQ ID NO: 77)
HRRLDKIEDERNLHEDFVFMKTIQRCNTGERSLSLLNCEEIKSQFEGFV
KDIMLNKEETKKENSFEMQKGDQNPQIAAHVISEASSKTTSVLQWAEKG
YYTMSNNLVTLENGKQLTVKRQGLYYIYAQVTFCSNREASSQAPFIASL
CLKSPGRFERILLRAANTHSSAKPCGQQSIHLGGVFELQPGASVFVNVT
DPSQVSHGTGFTSFGLLKL.

In embodiments, a chimeric protein comprises a variant of the extracellular domain of CD40L. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 77.

In embodiments, the second domain of a chimeric protein comprises an amino acid sequence that is at least 95% identical to the amino acid sequence of SEQ ID NO: 77.

One of ordinary skill may select variants of the known amino acid sequence of CD40L by consulting the literature, e.g., An, et al. Crystallographic and Mutational Analysis of the CD40-CD154 Complex and Its Implications for Receptor Activation, The Journal of Biological Chemistry 286, 11226-11235., which is incorporated by reference in its entirety.

In one aspect, the present disclosure relates to an isolated polynucleotide encoding a chimeric protein having a general structure of: N terminus—(a)-(b)-(c)—C terminus, wherein: (a) is a first domain comprising an extracellular domain of V-set and immunoglobulin domain-containing protein 8 (VSIG8), or a variant or a fragment thereof that capable of binding a VSIG8 ligand, (c) is a second domain comprising an extracellular domain of 4-1BB Ligand (4-1BBL), or a variant or a fragment thereof that capable of binding a 4-1BBL receptor, (b) is a linker adjoining the first and second domains, wherein the linker comprises at least one cysteine residue capable of forming a disulfide bond and/or comprises a hinge-CH2-CH3 Fc domain. In embodiments, the 4-1BBL receptor is 4-1BB.

In embodiments, a heterologous chimeric protein of the present disclosure comprises, or the isolated polynucleotide encoding the heterologous chimeric protein encodes, the extracellular domain of human V-set and immunoglobulin domain-containing protein 8 (VSIG8), which comprises the following amino acid sequence:

(SEQ ID NO: 78)
VRINGDGQEVLYLAEGDNVRLGCPYVLDPEDYGPNGLDIEWMQVNSDPA
HHRENVFLSYQDKRINHGSLPHLQQRVRFAASDPSQYDASINLMNLQVS
DTATYECRVKKTTMATRKVIVTVQARPAVPMCWTEGHMTYGNDVVLKCY
ASGGSQPLSYKWAKISGHHYPYRAGSYTSQHSYHSELSYQESFHSSINQ
GLNNGDLVLKDISRADDGLYQCTVANNVGYSVCVVEVKVSDSRRIG.

In embodiments, the present chimeric protein comprises a domain, e.g., the extracellular domain, of human VSIG8. The human VSIG8 comprises the amino acid sequence of SEQ ID NO: 78 (with the amino acid sequence of the extracellular domain comprising SEQ ID NO: 78).

In embodiments, the present chimeric proteins may comprise the extracellular domain of VSIG8 as described herein (e.g., SEQ ID NO: 78), or a variant or a functional fragment thereof. For instance, the chimeric protein may comprise a sequence of the extracellular domain of VSIG8 as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of the extracellular domain of VSIG8 as described herein.

VSIG8 derivatives can be constructed from available structural data. The structure of VSIG8 is understood based on domain structure and homology analyses. For example, Rubinstein et al., “Functional Classification of Immune Regulatory Proteins.” Structure 21(5): 766-776 (2013) disclosed that VSIG8 belongs to the JAM/CXR (Junctional adhesion molecule/cortical thymocyte marker in Xenopus) subfamily of Ig superfamily proteins. Moreover, without wishing to be bound by theory, the protein structure homology-model of VSIG8 is available at SWISS-MODEL repository. Bienert et al., “The SWISS-MODEL Repository—new features and functionality.” Nucleic Acids Research, 45(D1): D313-D319 (2017); Varadi et al., AlphaFold Protein Structure Database: massively expanding the structural coverage of protein-sequence space with high-accuracy models, Nucleic Acids Research, 50(D1); D439-D444 (2022), each of which is incorporated by reference in its entirety.

In embodiments, a heterologous chimeric protein of the present disclosure comprises, or the isolated polynucleotide encoding the heterologous chimeric protein encodes, the extracellular domain of human 4-1BBL, which comprises the following amino acid sequence:

(SEQ ID NO: 75)
ACPWAVSGARASPGSAASPRLREGPELSPDDPAGLLDLRQGMFAQLVAQ
NVLLIDGPLSWYSDPGLAGVSLTGGLSYKEDTKELVVAKAGVYYVFFQL
ELRRVVAGEGSGSVSLALHLQPLRSAAGAAALALTVDLPPASSEARNSA
FGFQGRLLHLSAGQRLGVHLHTEARARHAWQLTQGATVLGLFRVTPEIP
AGLPSPRSE.

In embodiments, a heterologous chimeric protein used in methods of the present disclosure comprises a variant of the extracellular domain of 4-1BBL As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 75.

In embodiments, the first domain of a heterologous chimeric protein comprises an amino acid sequence that is at least 95% identical to the amino acid sequence of SEQ ID NO: 75.

In embodiments, a heterologous chimeric protein comprises substantially the entire extracellular domain of 4-1BBL.

One of ordinary skill may select variants of the known amino acid sequence of 4-1BBL by consulting the literature, e.g., Won et al., “The structure of the trimer of human 4-1BB ligand is unique among members of the tumor necrosis factor superfamily.” J. Biol. Chem. 285: 9202-9210 (2010); Gilbreth et al., “Crystal structure of the human 4-1BB/4-1BBL complex.” J Biol Chem 293: 9880-9891 (2018); and Bitra et al., “Crystal structures of the human 4-1BB receptor bound to its ligand 4-1BBL reveal covalent receptor dimerization as a potential signaling amplifier.” J Biol Chem 293: 9958-9969 (2018); Li et al., (2018) Limited Cross-Linking of 4-1BB by 4-1BB Ligand and the Agonist Monoclonal Antibody Utomilumab, Cell Rep 25: 909-920.e4 and Uniprot structures 3B93, 3B94; each of which is incorporated by reference in its entirety.

In one aspect, the present disclosure relates to an isolated polynucleotide encoding a chimeric protein having a general structure of: N terminus—(a)-(b)-(c)—C terminus, wherein: (a) is a first domain comprising an extracellular domain of signal regulatory protein a (SIRPα), or a variant or a fragment thereof that is capable of binding a SIRPα ligand, (c) is a second domain comprising an extracellular domain of CD40 ligand (CD40L), or a variant or a fragment thereof that is capable of binding a CD40L receptor, (b) is a linker adjoining the first and second domains, wherein the linker comprises at least one cysteine residue capable of forming a disulfide bond and/or comprises a hinge-CH2-CH3 Fc domain. In embodiments, the SIRPα ligand is CD47. In embodiments, the CD40L receptor is CD40. In embodiments, the isolated polynucleotide is or comprises an mRNA. In embodiments, the isolated polynucleotide is or comprises an mRNA that is modified according to any of the embodiments disclosed herein.

In embodiments, a heterologous chimeric protein of the present disclosure comprises, or the isolated polynucleotide encoding the heterologous chimeric protein encodes, the extracellular domain of human signal regulatory protein a (SIRPα), which comprises the following amino acid sequence: In embodiments, a chimeric protein used in methods of the present disclosure comprises the extracellular domain of human SIRPα(CD172a) which comprises the following amino acid sequence:

(SEQ ID NO: 79)
EEELQVIQPDKSVLVAAGETATLRCTATSLIPVGPIQWFRGAGPGRELI
YNQKEGHFPRVTTVSDLTKRNNMDFSIRIGNITPADAGTYYCVKFRKGS
PDDVEFKSGAGTELSVRAKPSAPVVSGPAARATPQHTVSFTCESHGFSP
RDITLKWFKNGNELSDFQTNVDPVGESVSYSIHSTAKVVLTREDVHSQV
ICEVAHVTLQGDPLRGTANLSETIRVPPTLEVTQQPVRAENQVNVTCQV
RKFYPQRLQLTWLENGNVSRTETASTVTENKDGTYNWMSWLLVNVSAHR
DDVKLTCQVEHDGQPAVSKSHDLKVSAHPKEQGSNTAAENTGSNERNI
Y.

In embodiments, a chimeric protein used in methods of the present disclosure comprises a variant of the extracellular domain of SIRPα(CD172a). As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 79.

In embodiments, the variant of the extracellular domain of SIRPα(CD172a) has at least about 95% sequence identity with SEQ ID NO: 79.

One of ordinary skill may select variants of the known amino acid sequence of SIRPα(CD172a) by consulting the literature, e.g. LEE, et al., “Novel Structural Determinants of SIRPα that Mediate Binding of CD47,” The Journal of Immunology, 179, 7741-7750, 2007 and HATHERLEY, et al., “The Structure of the Macrophage Signal Regulatory Protein a (SIRPα) Inhibitory Receptor Reveals a Binding Face Reminiscent of That Used by T Cell Receptors,” The Journal Of Biological Chemistry, Vol. 282, No. 19, pp. 14567-14575, 2007, each of which is incorporated by reference in its entirety.

In embodiments, a heterologous chimeric protein of the present disclosure comprises, or the isolated polynucleotide encoding the heterologous chimeric protein encodes, the extracellular domain of human CD40L, which comprises the following amino acid sequence:

(SEQ ID NO: 77)
HRRLDKIEDERNLHEDFVFMKTIQRCNTGERSLSLLNCEEIKSQFEGFV
KDIMLNKEETKKENSFEMQKGDQNPQIAAHVISEASSKTTSVLQWAEKG
YYTMSNNLVTLENGKQLTVKRQGLYYIYAQVTFCSNREASSQAPFIASL
CLKSPGRFERILLRAANTHSSAKPCGQQSIHLGGVFELQPGASVFVNVT
DPSQVSHGTGFTSFGLLKL.

In embodiments, a chimeric protein comprises a variant of the extracellular domain of CD40L. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 77.

In embodiments, the second domain of a chimeric protein comprises an amino acid sequence that is at least 95% identical to the amino acid sequence of SEQ ID NO: 77.

One of ordinary skill may select variants of the known amino acid sequence of CD40L by consulting the literature, e.g., An, et al. Crystallographic and Mutational Analysis of the CD40-CD154 Complex and Its Implications for Receptor Activation, The Journal of Biological Chemistry 286, 11226-11235., which is incorporated by reference in its entirety.

Linkers In embodiments, the chimeric protein comprises a linker.

In embodiments, the linker comprising at least one cysteine residue capable of forming a disulfide bond. The at least one cysteine residue is capable of forming a disulfide bond between a pair (or more) of chimeric proteins. Without wishing to be bound by theory, such disulfide bond forming is responsible for maintaining a useful multimeric state of chimeric proteins. This allows for efficient production of the chimeric proteins; it allows for desired activity in vitro and in vivo.

In a chimeric protein of the present disclosure, the linker is a polypeptide selected from a flexible amino acid sequence, an IgG hinge region, or an antibody sequence.

In embodiments, the linker is derived from naturally-occurring multi-domain proteins or is an empirical linker as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the linker comprises a polypeptide. In embodiments, the polypeptide is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In embodiments, the linker is flexible.

In embodiments, the linker is rigid.

In embodiments, the linker is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines).

In embodiments, the linker comprises a hinge region of an antibody (e.g., of IgG, IgA, IgD, and IgE, inclusive of subclasses (e.g., IgG1, IgG2, IgG3, IgG4, IgA1, and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of C_{H1} to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in CH2. Id. The core hinge region of wild-type human IgG1 contains the sequence CPPC (SEQ ID NO: 24) which, when dimerized by disulfide bond formation, results in a cyclic octapeptide believed to act as a pivot, thus conferring flexibility. In embodiments, the present linker comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment.

For example, IgA1 contains five glycosylation sites within a 17-amino-acid segment of the hinge region, conferring resistance of the hinge region polypeptide to intestinal proteases, considered an advantageous property for a secretory immunoglobulin. In embodiments, the linker of the present disclosure comprises one or more glycosylation sites.

In embodiments, the linker comprises an Fc domain of an antibody (e.g., of IgG, IgA, IgD, and IgE, inclusive of subclasses (e.g., IgG1, IgG2, IgG3, and IgG4, and IgA1 and IgA2)).

In a chimeric protein of the present disclosure, the linker comprises a hinge-CH2-CH3 Fc domain derived from IgG4. In embodiments, the linker comprises a hinge-CH2-CH3 Fc domain derived from a human IgG4. In embodiments, the linker comprises an amino acid sequence that is at least 95% identical to the amino acid sequence of any one of SEQ ID NO: 1 to SEQ ID NO: 3, e.g., at least 95% identical to the amino acid sequence of SEQ ID NO: 2. In embodiments, the linker comprises one or more joining linkers, such joining linkers independently selected from SEQ ID NO: 4 to SEQ ID NO: 50 (or a variant thereof). In embodiments, the linker comprises two or more joining linkers each joining linker independently selected from SEQ ID NO: 4 to SEQ ID NO: 50 (or a variant thereof); wherein one joining linker is N terminal to the hinge-CH2-CH3 Fc domain and another joining linker is C terminal to the hinge-CH2-CH3 Fc domain.

In embodiments, the linker comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the present chimeric proteins.

In embodiments, the Fc domain in a linker contains one or more amino acid substitutions at amino acid residue 250, 252, 254, 256, 308, 309, 311, 416, 428, 433 or 434 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference), or equivalents thereof. In embodiments, the amino acid substitution at amino acid residue 250 is a substitution with glutamine. In embodiments, the amino acid substitution at amino acid residue 252 is a substitution with tyrosine, phenylalanine, tryptophan or threonine. In embodiments, the amino acid substitution at amino acid residue 254 is a substitution with threonine. In embodiments, the amino acid substitution at amino acid residue 256 is a substitution with serine, arginine, glutamine, glutamic acid, aspartic acid, or threonine. In embodiments, the amino acid substitution at amino acid residue 308 is a substitution with threonine. In embodiments, the amino acid substitution at amino acid residue 309 is a substitution with proline. In embodiments, the amino acid substitution at amino acid residue 311 is a substitution with serine. In embodiments, the amino acid substitution at amino acid residue 385 is a substitution with arginine, aspartic acid, serine, threonine, histidine, lysine, alanine or glycine. In embodiments, the amino acid substitution at amino acid residue 386 is a substitution with threonine, proline, aspartic acid, serine, lysine, arginine, isoleucine, or methionine. In embodiments, the amino acid substitution at amino acid residue 387 is a substitution with arginine, proline, histidine, serine, threonine, or alanine. In embodiments, the amino acid substitution at amino acid residue 389 is a substitution with proline, serine or asparagine. In embodiments, the amino acid substitution at amino acid residue 416 is a substitution with serine. In embodiments, the amino acid substitution at amino acid residue 428 is a substitution with leucine. In embodiments, the amino acid substitution at amino acid residue 433 is a substitution with arginine, serine, isoleucine, proline, or glutamine. In embodiments, the amino acid substitution at amino acid residue 434 is a substitution with histidine, phenylalanine, or tyrosine.

In embodiments, the Fc domain linker (e.g., comprising an IgG constant region) comprises one or more mutations such as substitutions at amino acid residue 252, 254, 256, 433, 434, or 436 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). In embodiments, the IgG constant region includes a triple M252Y/S254T/T256E mutation or YTE mutation. In embodiments, the IgG constant region includes a triple H433K/N434F/Y436H mutation or KFH mutation. In embodiments, the IgG constant region includes an YTE and KFH mutation in combination.

In embodiments, the linker comprises an IgG constant region that contains one or more mutations at amino acid residues 250, 253, 307, 310, 380, 428, 433, 434, and 435 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). Illustrative mutations include T250Q, M428L, T307A, E380A, I253A, H310A, M428L, H433K, N434A, N434F, N434S, and H435A. In embodiments, the IgG constant region comprises a M428L/N434S mutation or LS mutation. In embodiments, the IgG constant region comprises a T250Q/M428L mutation or QL mutation. In embodiments, the IgG constant region comprises an N434A mutation. In embodiments, the IgG constant region comprises a T307A/E380A/N434A mutation or AAA mutation. In embodiments, the IgG constant region comprises an I253A/H310A/H435A mutation or IHH mutation. In embodiments, the IgG constant region comprises a H433K/N434F mutation. In embodiments, the IgG constant region comprises a M252Y/S254T/T256E and a H433K/N434F mutation in combination.

Additional exemplary mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al. Nature (2014) 514:642-645, Grevys et al. Journal of Immunology. (2015), 194(11):5497-508, and U.S. Pat. No. 7,083,784, the entire contents of which are hereby incorporated by reference.

An illustrative Fc stabilizing mutant is S228P. Illustrative Fc half-life extending mutants are T250Q, M428L, V308T, L309P, and Q311S and the present linkers may comprise 1, or 2, or 3, or 4, or 5 of these mutants.

In embodiments, the chimeric protein binds to FcRn with high affinity. In embodiments, the chimeric protein may bind to FcRn with a K_{D} of about 1 nM to about 80 nM. For example, the chimeric protein may bind to FcRn with a K_{D} of about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 71 nM, about 72 nM, about 73 nM, about 74 nM, about 75 nM, about 76 nM, about 77 nM, about 78 nM, about 79 nM, or about 80 nM. In embodiments, the chimeric protein may bind to FcRn with a K_{D} of about 9 nM. In embodiments, the chimeric protein does not substantially bind to other Fc receptors (e.g. other than FcRn) with effector function.

In embodiments, the Fc domain in a linker has the amino acid sequence of SEQ ID NO: 1 (see Table 1, below), or at least 90%, or 93%, or 95%, or 97%, or 98%, or 99% identity thereto. In embodiments, mutations are made to SEQ ID NO: 1 to increase stability and/or half-life. For instance, in embodiments, the Fc domain in a linker comprises the amino acid sequence of SEQ ID NO: 2 (see Table 1, below), or at least 90%, or 93%, or 95%, or 97%, or 98%, or 99% identity thereto. For instance, in embodiments, the Fc domain in a linker comprises the amino acid sequence of SEQ ID NO: 3 (see Table 1, below), or at least 90%, or 93%, or 95%, or 97%, or 98%, or 99% identity thereto.

Further, one or more joining linkers may be employed to connect an Fc domain in a linker (e.g., one of SEQ ID NO: 1, SEQ ID NO: 2, SEQ ID NO: 3 or at least 90%, or 93%, or 95%, or 97%, or 98%, or 99% identity thereto) and the extracellular domains. For example, any one of SEQ ID NO: 4, SEQ ID NO: 5, SEQ ID NO: 6, SEQ ID NO: 7, SEQ ID NO: 8, SEQ ID NO: 9, or variants thereof may connect an extracellular domain as disclosed herein and an Fc domain in a linker as disclosed herein. Optionally, any one of SEQ ID NO: 4 to SEQ ID NO: 50, or variants thereof are located between an extracellular domain as disclosed herein and an Fc domain as disclosed herein.

In embodiments, the present chimeric proteins may comprise variants of the joining linkers disclosed in Table 1, below. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NO: 4 to SEQ ID NO: 50.

In embodiments, the first and second joining linkers may be different or they may be the same.

Without wishing to be bound by theory, including a linker comprising at least a part of an Fc domain in a chimeric protein, helps avoid formation of insoluble and, likely, non-functional protein concatenated oligomers and/or aggregates. This is in part due to the presence of cysteines in the Fc domain which are capable of forming disulfide bonds between chimeric proteins.

In embodiments, a chimeric protein may comprise one or more joining linkers, as disclosed herein, and lack a Fc domain linker, as disclosed herein.

In embodiments, the first and/or second joining linkers are independently selected from the amino acid sequences of SEQ ID NO: 4 to SEQ ID NO: 50 and are provided in Table 1 below:

TABLE 1
Illustrative linkers (Fc domain linkers and joining linkers)

| SEQ ID | |
|---|---|
| NO. | Sequence |
| 1 | APEFLGGPSVFLFPPKPKDTLMISRTPEVTCVVVDVSQEDPEVQFNWYVDGVEVHNAKTKPR |
| | EEQFNSTYRVVSVLTVLHQDWLSGKEYKCKVSSKGLPSSIEKTISNATGQPREPQVYTLPPSQ |
| | EEMTKNQVSLTCLVKGFYPSDIAVEWESNGQPENNYKTTPPVLDSDGSFFLYSRLTVDKSSW |
| | QEGNVFSCSVMHEALHNHYTQKSLSLSLGK |
| 2 | APEFLGGPSVFLFPPKPKDQLMISRTPEVTCVVVDVSQEDPEVQFNWYVDGVEVHNAKTKPR |
| | EEQFNSTYRVVSVLTTPHSDWLSGKEYKCKVSSKGLPSSIEKTISNATGQPREPQVYTLPPSQ |
| | EEMTKNQVSLTCLVKGFYPSDIAVEWESNGQPENNYKTTPPVLDSDGSFFLYSRLTVDKSSW |
| | QEGNVFSCSVLHEALHNHYTQKSLSLSLGK |
| 3 | APEFLGGPSVFLFPPKPKDQLMISRTPEVTCVVVDVSQEDPEVQFNWYVDGVEVHNAKTKPR |
| | EEQFNSTYRVVSVLTVLHQDWLSGKEYKCKVSSKGLPSSIEKTISNATGQPREPQVYTLPPSQ |
| | EEMTKNQVSLTCLVKGFYPSDIAVEWESNGQPENNYKTTPPVLDSDGSFFLYSRLTVDKSRW |
| | QEGNVFSCSVLHEALHNHYTQKSLSLSLGK |
| 4 | EPKSCDKTHTCPPCPAPELLGGPSVFLFPPKPKDTLMISRTPEVTCVVVDVSHEDPEVKFNWY |
| | VDGVEVHNAKTKPREEQYASTYRVVSVLTVLHQDWLNGKEYKCKVSNKALPAPIEKTISKAKG |
| | QPREPQVYTLPPSRDELTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| 5 | EPKSCDKTHTCPPCPAPEAAGGPSVFLFPPKPKDTLMISRTPEVTCVVVDVSHEDPEVKFNW |
| | YVDGVEVHNAKTKPREEQYASTYRVVSVLTVLHQDWLNGKEYKCKVSNKALPAPIEKTISKAK |
| | GQPREPQVYTLPPSRDELTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| 6 | SKYGPPCPSCP |
| 7 | SKYGPPCPPCP |
| 8 | SKYGPP |
| 9 | IEGRMD |
| 10 | GGGVPRDCG |
| 11 | IEGRMDGGGGAGGGG |
| 12 | GGGSGGGS |
| 13 | GGGSGGGGSGGG |
| 14 | EGKSSGSGSESKST |
| 15 | GGSG |
| 16 | GGSGGGSGGGSG |
| 17 | EAAAKEAAAKEAAAK |
| 18 | EAAAREAAAREAAAREAAAR |
| 19 | GGGGSGGGGSGGGGSAS |
| 20 | GGGGAGGGG |
| 21 | GS or GGS or LE |
| 22 | GSGSGS |
| 23 | GSGSGSGSGS |
| 24 | GGGGSAS |
| 25 | APAPAPAPAPAPAPAPAPAP |
| 26 | CPPC |
| 27 | GGGGS |
| 28 | GGGGSGGGGS |
| 29 | GGGGSGGGGSGGGGS |
| 30 | GGGGGGGGSGGGGSGGGGS |
| 31 | GGGGSGGGGSGGGGSGGGGSGGGGS |
| 32 | GGGGSGGGGSGGGGSGGGGSGGGGSGGGGS |
| 33 | GGGGSGGGGSGGGGSGGGGSGGGGSGGGGSGGGGS |
| 34 | GGGGSGGGGSGGGGSGGGGSGGGGSGGGGSGGGGSGGGGS |
| 35 | GGSGGSGGGGGGGGS |
| 36 | GGGGGGGG |
| 37 | GGGGGG |
| 38 | EAAAK |
| 39 | EAAAKEAAAK |
| 40 | EAAAKEAAAKEAAAK |
| 41 | AEAAAKEAAAKA |
| 42 | AEAAAKEAAAKEAAAKA |
| 43 | AEAAAKEAAAKEAAAKEAAAKA |
| 44 | AEAAAKEAAAKEAAAKEAAAKEAAAKA |
| 45 | AEAAAKEAAAKEAAAKEAAAKALEAEAAAKEAAAKEAAAKEAAAKA |
| 46 | PAPAP |
| 47 | KESGSVSSEQLAQFRSLD |
| 48 | GSAGSAAGSGEF |
| 49 | GGGSE |
| 50 | GSESG |
| 51 | GSEGS |
| 52 | GEGGSGEGSSGEGSSSEGGGSEGGGSEGGGSEGGS |

In embodiments, the joining linker substantially comprises glycine and serine residues (e.g., about 30%, or about 40%, or about 50%, or about 60%, or about 70%, or about 80%, or about 90%, or about 95%, or about 97%, or about 98%, or about 99%, or about 100% glycines and serines). For example, in embodiments, the joining linker is (Gly₄Ser)ₙ, where n is from about 1 to about 8, e.g., 1, 2, 3, 4, 5, 6, 7, or 8 (SEQ ID NO: 27 to SEQ ID NO: 34, respectively). In embodiments, the joining linker sequence is GGSGGSGGGGSGGGGS (SEQ ID NO: 35). Additional illustrative joining linkers include, but are not limited to, linkers having the sequence LE, (EAAAK)ₙ (n=1-3) (SEQ ID NO: 38 to SEQ ID NO: 40), A(EAAAK)ₙA (n=2-5) (SEQ ID NO: 41 to SEQ ID NO: 44), A(EAAAK)₄ALEA(EAAAK)₄A (SEQ ID NO: 45), PAPAP (SEQ ID NO: 46), KESGSVSSEQLAQFRSLD (SEQ ID NO: 47), GSAGSAAGSGEF (SEQ ID NO: 48), and (XP)ₙ, with X designating any amino acid, e.g., Ala, Lys, or Glu. In embodiments, thejoining linker is GGS. In embodiments, a joining linker has the sequence (Gly)ₙ where n is any number from 1 to 100, for example: (Gly)₈ (SEQ ID NO: 36) and (Gly)₆ (SEQ ID NO: 37).

In embodiments, the joining linker is one or more of GGGSE (SEQ ID NO: 49), GSESG (SEQ ID NO: 50), GSEGS (SEQ ID NO: 51), GEGGSGEGSSGEGSSSEGGGSEGGGSEGGGSEGGS (SEQ ID NO: 52), and a joining linker of randomly placed G, S, and E every 4 amino acid intervals.

In embodiments, where a chimeric protein comprises a first domain, one joining linker preceding an Fc domain, a second joining linker following the Fc domain, and a second domain, the chimeric protein may comprise the following structure:

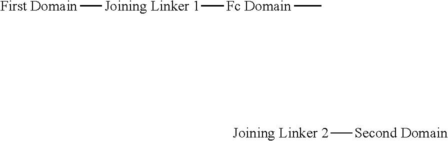

The combination of a first joining linker, an Fc Domain linker, and a second joining linker is referend to herein as a “modular linker”. In embodiments, a chimeric protein comprises a modular linker as shown in Table 2:

TABLE 2
Illustrative modular linkers

| | | | Modular Linker = Joining |
|---|---|---|---|
| Joining Linker | | Joining Linker | Linker 1 + Fc + Joining |
| 1 | Fc | 2 | Linker 2 |
| SKYGPPCPSC | APEFLGGPSVFLFPPKPKDTL | IEGRMD | SKYGPPCPSCPAPEFLGGPSV |
| P | MISRTPEVTCVVVDVSQEDPE | (SEQ ID NO: | FLFPPKPKDTLMISRTPEVTCV |
| (SEQ ID NO: 6) | VQFNWYVDGVEVHNAKTKPR | 9) | WVDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVMHEALHNHYTQK | | LTVDKSSWQEGNVFSCSVMH |
| | SLSLSLGK (SEQ ID NO: 1) | | EALHNHYTQKSLSLSLGKIEGR |
| | | | MD (SEQ ID NO: 53) |
| SKYGPPCPSC | APEFLGGPSVFLFPPKPKDQL | IEGRMD | SKYGPPCPSCPAPEFLGGPSV |
| P | MISRTPEVTCVVVDVSQEDPE | (SEQ ID NO: | FLFPPKPKDQLMISRTPEVTCV |
| (SEQ ID NO: 9) | VQFNWYVDGVEVHNAKTKPR | 9) | WDVSQEDPEVQFNWYVDGV |
| | EEQFNSTYRVVSVLTTPHSDW | | EVHNAKTKPREEQFNSTYRVV |
| | LSGKEYKCKVSSKGLPSSIEKT | | SVLTTPHSDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVLHEALHNHYTQKS | | LTVDKSSWQEGNVFSCSVLHE |
| | LSLSLGK (SEQ ID NO: 2) | | ALHNHYTQKSLSLSLGKIEGR |
| | | | MD (SEQ ID NO: 54) |
| SKYGPPCPSC | APEFLGGPSVFLFPPKPKDQL | IEGRMD | SKYGPPCPSCPAPEFLGGPSV |
| P | MISRTPEVTCVVVDVSQEDPE | (SEQ ID NO: | FLFPPKPKDQLMISRTPEVTCV |
| (SEQ ID NO: 6) | VQFNWYVDGVEVHNAKTKPR | 9) | WDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSRWQE | | NNYKTTPPVLDSDGSFFLYSR |
| | GNVFSCSVLHEALHNHYTQKS | | LTVDKSRWQEGNVFSCSVLHE |
| | LSLSLGK (SEQ ID NO: 5) | | ALHNHYTQKSLSLSLGKIEGR |
| | | | MD (SEQ ID NO: 55) |
| SKYGPPCPPC | APEFLGGPSVFLFPPKPKDTL | IEGRMD | SKYGPPCPPCPAPEFLGGPSV |
| P | MISRTPEVTCVVVDVSQEDPE | (SEQ ID NO: | FLFPPKPKDTLMISRTPEVTCV |
| (SEQ ID NO: 7) | VQFNWYVDGVEVHNAKTKPR | 9) | WVDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVMHEALHNHYTQK | | LTVDKSSWQEGNVFSCSVMH |
| | SLSLSLGK (SEQ ID NO: 1) | | EALHNHYTQKSLSLSLGKIEGR |
| | | | MD (SEQ ID NO: 56) |
| SKYGPPCPPC | APEFLGGPSVFLFPPKPKDQL | IEGRMD | SKYGPPCPPCPAPEFLGGPSV |
| P | MISRTPEVTCVVVDVSQEDPE | (SEQ ID NO: | FLFPPKPKDQLMISRTPEVTCV |
| (SEQ ID NO: 7) | VQFNWYVDGVEVHNAKTKPR | 9) | WVDVSQEDPEVQFNWYVDGV |
| | EEQFNSTYRVVSVLTTPHSDW | | EVHNAKTKPREEQFNSTYRVV |
| | LSGKEYKCKVSSKGLPSSIEKT | | SVLTTPHSDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVLHEALHNHYTQKS | | LTVDKSSWQEGNVFSCSVLHE |
| | LSLSLGK (SEQ ID NO: 2) | | ALHNHYTQKSLSLSLGKIEGR |
| | | | MD (SEQ ID NO: 57) |
| SKYGPPCPPC | APEFLGGPSVFLFPPKPKDQL | IEGRMD | SKYGPPCPPCPAPEFLGGPSV |
| P | MISRTPEVTCVVVDVSQEDPE | (SEQ ID NO: | FLFPPKPKDQLMISRTPEVTCV |
| (SEQ ID NO: 7) | VQFNWYVDGVEVHNAKTKPR | 9) | VVDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSRWQE | | NNYKTTPPVLDSDGSFFLYSR |
| | GNVFSCSVLHEALHNHYTQKS | | LTVDKSRWQEGNVFSCSVLHE |
| | LSLSLGK (SEQ ID NO: 3) | | ALHNHYTQKSLSLSLGKIEGR |
| | | | MD (SEQ ID NO: 58) |

In embodiments, the present chimeric proteins may comprise variants of the modular linkers disclosed in Table 2, above. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NO: 53 to SEQ ID NO: 58.

In embodiments, the linker may be flexible, including without limitation highly flexible. In embodiments, the linker may be rigid, including without limitation a rigid alpha helix. Characteristics of illustrative joining linkers is shown below in Table 3:

TABLE 3
Characteristics of illustrative joining linkers

| Joining Linker Sequence | Characteristics |
|---|---|
| SKYGPPCPPCP (SEQ ID NO: 7) | IgG4 Hinge Region |
| IEGRMD (SEQ ID NO: 9) | Linker |
| GGGVPRDCG (SEQ ID NO: 10) | Flexible |
| GGGSGGGS (SEQ ID NO: 12) | Flexible |
| GGGSGGGGSGGG (SEQ ID NO: 13) | Flexible |
| EGKSSGSGSESKST (SEQ ID NO: 14) | Flexible + soluble |
| GGSG (SEQ ID NO: 15) | Flexible |
| GGSGGGSGGGSG (SEQ ID NO: 16) | Flexible |
| EAAAKEAAAKEAAAK (SEQ ID NO: 17) | Rigid Alpha Helix |
| EAAAREAAAREAAAREAAAR (SEQ ID NO: 18) | Rigid Alpha Helix |
| GGGGSGGGGSGGGGSAS (SEQ ID NO: 19) | Flexible |
| GGGGAGGGG (SEQ ID NO: 20) | Flexible |
| GS (SEQ ID NO: 21) | Highly flexible |
| GSGSGS (SEQ ID NO: 22) | Highly flexible |
| GSGSGSGSGS (SEQ ID NO: 23) | Highly flexible |
| GGGGSAS (SEQ ID NO: 24) | Flexible |
| APAPAPAPAPAPAPAPAPAP (SEQ ID NO: 25) | Rigid |

In embodiments, the linker may be functional. For example, without limitation, the linker may function to improve the folding and/or stability, improve the expression, improve the pharmacokinetics, and/or improve the bioactivity of the present chimeric protein. In another example, the linker may function to target the chimeric protein to a particular cell type or location.

In embodiments, a chimeric protein comprises only one joining linkers.

In embodiments, a chimeric protein lacks joining linkers.

### The Chimeric Proteins and Isolated Polynucleotide Coding the Chimeric Proteins

In one aspect, the present disclosure relates to an isolated polynucleotide encoding a chimeric protein having a general structure of: N terminus—(a)-(b)-(c)—C terminus, wherein: (a) is a first domain comprising an extracellular domain of T cell immunoreceptor with Ig and ITIM domains (TIGIT), or a variant or a fragment thereof that is capable of binding a TIGIT ligand, (c) is a second domain comprising an extracellular domain of glucocorticoid-induced TNFR-related protein ligand (GITRL), or a variant or a fragment thereof that is capable of binding a GITRL receptor, or LIGHT (homologous to lymphotoxin, exhibits inducible expression and competes with HSV glycoprotein D for binding to herpesvirus entry mediator, a receptor expressed on T lymphocytes), or a variant or a fragment thereof that is capable of binding a LIGHT receptor, (b) is a linker adjoining the first and second domains, wherein the linker comprises at least one cysteine residue capable of forming a disulfide bond and/or comprises a hinge-CH2-CH3 Fc domain. In embodiments, the TIGIT ligand is selected from CD155/PVR, Nectin-2, Nectin-3 and Nectin-4. In embodiments, the GITRL receptor is glucocorticoid-induced TNFR-related protein (GITR). In embodiments, the LIGHT receptor is TNFRSF3/LTBR. In embodiments, the isolated polynucleotide is or comprises an mRNA. In embodiments, the isolated polynucleotide is or comprises an mRNA that is modified according to any of the embodiments disclosed herein.

In embodiments, the chimeric protein comprises a portion of TIGIT, a joining linker preceding an Fc domain, the Fc domain, a joining linker following the Fc domain, and a portion GITRL, and the chimeric protein may comprise the following structure:

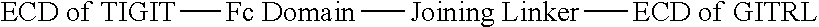

In embodiments, the chimeric protein comprises: an extracellular domain of TIGIT, capable of binding a TIGIT ligand, and comprising an amino acid sequence that is at least about 90%, or at least about 95%, or at least about 97%, or at least about 98%, or at least about 99% identical to the amino acid sequence of SEQ ID NO: 59; an extracellular domain of GITRL, capable of binding a GITRL receptor, and comprising an amino acid sequence that is at least about 90%, or at least about 95%, or at least about 97%, or at least about 98%, or at least about 99% identical to the amino acid sequence of SEQ ID NO: 60; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain.

An illustrative TIGIT-Fc-GITRL chimeric protein has the following sequence (the extracellular domain of TIGIT is shown by an underline, a linker comprising a mutant Fc domain of human IgG1 is shown in boldface font, with mutations shown by an underline, a joining linker is shown in an underlined-boldface-italic font, and the extracellular domain of GITRL is shown in an italics font):

(SEQ ID NO: 80)
MMTGTIETTGNISAEKGGSIILQCHLSSTTAQVTQVNWEQQDQLLAICNADLGWHISPSFKDRVAPGPGLGL
TLQSLTVNDTGEYFCIYHTYPDGTYTGRIFLEVLESSVAEHGARFQIPSKYGPPCPPCPAPEFLGGPSVFLF
PPKPKDQLMISRTPEVTCVVVDVSQEDPEVQFNWYVDGVEVHNAKTKPREEQFNSTYRVVSVLTVLHQD
WLSGKEYKCKVSSKGLPSSIEKTISNATGQPREPQVYTLPPSQEEMTKNQVSLTCLVKGFYPSDIAVEWES
NGQPENNYKTTPPVLDSDGSFFLYSRLTVDKSRWQEGNVFSCSVLHEALHNHYTQKSLSLSLGKIEGRMD
QLETAKEPCMAKFGPLPSKWQMASSEPPCVNKVSDWKLEILQNGLYLIYGQVAPNANYNDVAPFEVRLYK
NKDMIQTLTNKSKIQNVGGTYELHVGDTIDLIFNSEHQVLKNNTYWGIILLANPQFIS.

In embodiments, the chimeric protein comprises a variant of the TIGIT-Fc-GITRL chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 80.

In embodiments, the chimeric protein comprises a portion of TIGIT, a joining linker preceding an Fc domain, the Fc domain, a joining linker following the Fc domain, and a portion LIGHT, and the chimeric protein may comprise the following structure:

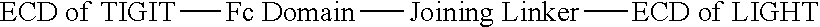

In embodiments, the chimeric protein comprises: an extracellular domain of TIGIT, capable of binding a TIGIT ligand, and comprising an amino acid sequence that is at least about 90%, or at least about 95%, or at least about 97%, or at least about 98%, or at least about 99% identical to the amino acid sequence of SEQ ID NO: 59; an extracellular domain of LIGHT, capable of binding a LIGHT receptor, and comprising an amino acid sequence that is at least about 90%, or at least about 95%, or at least about 97%, or at least about 98%, or at least about 99% identical to the amino acid sequence of SEQ ID NO: 61; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain.

An illustrative TIGIT-Fc-LIGHT chimeric protein has the following sequence (the extracellular domain (ECD) of human TIGIT is indicated by underline, a variant IgG4 CH2-CH3-Fc domain is shown in an italic font, joining linkers are shown in a boldface font, and the extracellular domain (ECD) of human LIGHT is indicated by an underlined, italic font):

(SEQ ID NO: 81)
MMTGTIETTGNISAEKGGSIILQCHLSSTTAQVTQVNWEQQDQLLAICNADLGWHISPSFKDRVAPGPGLGL
TLQSLTVNDTGEYFCIYHTYPDGTYTGRIFLEVLESSVAEHGARFQIPSKYGPPCPPCPAPEFLGGPSVFLF
PPKPKDQLMISRTPEVTCVVVDVSQEDPEVQFNWYVDGVEVHNAKTKPREEQFNSTYRVVSVLTVLHQD
WLSGKEYKCKVSSKGLPSSIEKTISNATGQPREPQVYTLPPSQEEMTKNQVSLTCLVKGFYPSDIAVEWES
NGQPENNYKTTPPVLDSDGSFFLYSRLTVDKSRWQEGNVFSCSVLHEALHNHYTQKSLSLSLGKIEGRMD
LQLHWRLGEMVTRLPDGPAGSWEQLIQERRSHEVNPAAHLTGANSSLTGSGGPLLWETQLGLAFLRGLS
YHDGALVVTKAGYYYIYSKVQLGGVGCPLGLASTITHGLYKRTPRYPEELELLVSQQSPCGRATSSSRVW
WDSSFLGGVVHLEAGEKVVVRVLDERLVRLRDGTRSYFGAFMV.

In embodiments, the chimeric protein comprises a variant of the TIGIT-Fc-LIGHT chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 81.

In one aspect, the present disclosure relates to an isolated polynucleotide encoding a chimeric protein having a general structure of: N terminus—(a)-(b)-(c)—C terminus, wherein: (a) is a first domain comprising an extracellular domain of tumor necrosis factor (TNF) receptor 2 (TNFR2), or a variant or a fragment thereof that is capable of binding a TNFR2 ligand, (c) is a second domain comprising an extracellular domain selected from CLEC7a, or a variant or a fragment thereof that capable of binding a CLEC7a ligand, DC-SIGN(CD209), or a variant or a fragment thereof that capable of binding a DC-SIGN(CD209) ligand, DECTIN2(CLEC6A), or a variant or a fragment thereof that capable of binding a DECTIN2(CLEC6A) ligand, Langerin(CD207,CLC4K), or a variant or a fragment thereof that capable of binding a Langerin(CD207,CLC4K) ligand, CD69, or a variant or a fragment thereof that capable of binding a CD69 ligand, and TGF-beta, or a variant or a fragment thereof that capable of binding a TGF-beta receptor, (b) is a linker adjoining the first and second domains, wherein the linker comprises at least one cysteine residue capable of forming a disulfide bond and/or comprises a hinge-CH2-CH3 Fc domain. In embodiments, the TNFR2 ligand is TNFα. In embodiments, the CLEC7a ligand is a beta-1,3-linked and/or beta-1,6-linked glucan. In embodiments, the DC-SIGN(CD209) ligand is a Intercellular Adhesion Molecule 2 (ICAM2) and/or Intercellular Adhesion Molecule 3 (ICAM3). In embodiments, the DECTIN2(CLEC6A) ligand is an alpha-mannan. In embodiments, the Langerin(CD207,CLC4K) ligand is a sulfated glycan, a mannosylated glycan, a keratan sulfate (KS) and/or a beta-glucan. In embodiments, the CD69 ligand is Galectin-1 (Gal-1) or the S100A8/S100A9 complex. In embodiments, the TGF-beta receptor is TGFBR1 and TGFBR2. In embodiments, the isolated polynucleotide is or comprises an mRNA. In embodiments, the isolated polynucleotide is or comprises an mRNA that is modified according to any of the embodiments disclosed herein.

In embodiments, where the chimeric protein comprises a portion of TNFR2, a joining linker preceding an Fc domain, the Fc domain, a joining linker following the Fc domain, and a portion Clec7a, the chimeric protein may comprise the following structure:

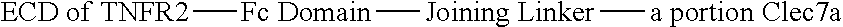

In embodiments, the chimeric protein comprises: an extracellular domain of TNFR2 comprising an amino acid sequence that is at least about 90%, or at least about 95%, or at least about 97%, or at least about 98%, or at least about 99% identical to the amino acid sequence of SEQ ID NO: 62; a portion of Clec7a capable of binding a beta-1,3-linked and/or beta-1,6-linked glucan, and comprising an amino acid sequence that is at least about 90%, or at least about 95%, or at least about 97%, or at least about 98%, or at least about 99% identical to the amino acid sequence of SEQ ID NO: 63 or SEQ ID NO: 64; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain.

An illustrative TNFR2-Fc-Clec7a chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the extracellular domain of Clec7a is shown in an italics font):

(SEQ ID NO: 82)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDVVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDEPKSCDKTHTCPPCPAPEAAGGPSVFLFPPKPKDTL
MISRTPEVTCVVVDVSHEDPEVKFNWYVDGVEVHNAKTKPREEQYASTYRVVSVLTVLHQDWLNGKE
YKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGKIEGRMD
TMAIWRSNSGSNTLENGYFLSRNKENHSQPTQSSLEDSVTPTKAVKTTGVLSSPCPPNWIIYEKSCYLF
SMSLNSWDGSKRQCWQLGSNLLKIDSSNELGFIVKQVSSQPDNSFWIGLSRPQTEVPWLWEDGSTFS
SNLFQIRTTATQENPSPNCVWIHVSVIYDQLCSVPSYSICEKKFSM.

In embodiments, the chimeric protein comprises a variant of the TNFR2-Fc-Clec7a chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 82.

An illustrative TNFR2-Fc-Clec7a chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the C-type lectin binding domain (CLD) of Clec7a is shown in an italics font):

(SEQ ID NO: 83)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDWVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDEPKSCDKTHTCPPCPAPEAAGGPSVFLFPPKPKDTL
MISRTPEVTCVVVDVSHEDPEVKFNWYVDGVEVHNAKTKPREEQYASTYRVVSVLTVLHQDWLNGKE
YKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGKIEGRMD
SSPCPPNWIIYEKSCYLFSMSLNSWDGSKRQCWQLGSNLLKIDSSNELGFIVKQVSSQPDNSFWIGLSR
PQTEVPWLWEDGSTFSSNLFQIRTTATQENPSPNCVWIHVSVIYDQLCSVPSYSICEKKFSM.

In embodiments, the chimeric protein comprises a variant of the TNFR2-Fc-Clec7a chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 83.

In embodiments, where the chimeric protein comprises an extracellular domain (ECD) of TNFR2, a joining linker preceding an Fc domain, the Fc domain, a joining linker following the Fc domain, and a portion of langerin, the chimeric protein may comprise the following structure:

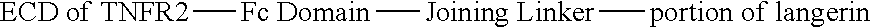

In embodiments, the chimeric protein comprises: an extracellular domain of TNFR2 comprising an amino acid sequence that is at least about 90%, or at least about 95%, or at least about 97%, or at least about 98%, or at least about 99% identical to the amino acid sequence of SEQ ID NO: 62; a portion of langerin capable of binding a ligand comprising a sulfated glycan, a mannosylated glycan, a keratan sulfate (KS) and/or a beta-glucan, and comprising an amino acid sequence that is at least about 90%, or at least about 95%, or at least about 97%, or at least about 98%, or at least about 99% identical to the amino acid sequence of SEQ ID NO: 69 or SEQ ID NO: 70; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3 or SEQ ID NO: 74. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain.

An illustrative TNFR2-Fc-langerin chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the extracellular domain of langerin is shown in an italics font):

(SEQ ID NO: 84)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDWVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDEPKSCDKTHTCPPCPAPEAAGGPSVFLFPPKPKDTL
MISRTPEVTCVVVDVSHEDPEVKFNWYVDGVEVHNAKTKPREEQYASTYRVVSVLTVLHQDWLNGKE
YKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGKIEGRMD
PRFMGTISDVKTNVQLLKGRVDNISTLDSEIKKNSDGMEAAGVQIQMVNESLGYVRSQFLKLKTSVEKA
NAQIQILTRSWEEVSTLNAQIPELKSDLEKASALNTKIRALQGSLENMSKLLKRQNDILQVVSQGWKYFK
GNFYYFSLIPKTWYSAEQFCVSRNSHLTSVTSESEQEFLYKTAGGLIYWIGLTKAGMEGDWSWVDDTP
FNKVQSVRFWIPGEPNNAGNNEHCGNIKAPSLQAWNDAPCDKTFLFICKRPYVPSEP.

In embodiments, the chimeric protein comprises a variant of the TNFR2-Fc-langerin chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 84.

An illustrative TNFR2-Fc-langerin chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the C-type lectin binding domain (CLD) of langerin is shown in an italics font):

(SEQ ID NO: 85)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDVVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDEPKSCDKTHTCPPCPAPEAAGGPSVFLFPPKPKDTL
MISRTPEVTCVVVDVSHEDPEVKFNWYVDGVEVHNAKTKPREEQYASTYRVVSVLTVLHQDWLNGKE
YKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGKIEGRMD
QVVSQGWKYFKGNFYYFSLIPKTWYSAEQFCVSRNSHLTSVTSESEQEFLYKTAGGLIYWIGLTKAGME
GDWSWVDDTPFNKVQSVRFWIPGEPNNAGNNEHCGNIKAPSLQAWNDAPCDKTFLFICKRPYVPSEP.

In embodiments, the chimeric protein comprises a variant of the TNFR2-Fc-langerin chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 85.

In embodiments, where the chimeric protein comprises an extracellular domain (ECD) of TNFR2, a joining linker preceding an Fc domain, the Fc domain, a joining linker following the Fc domain, and a portion of DC-SIGN the chimeric protein may comprise the following structure:

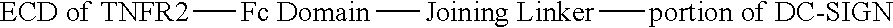

In embodiments, the chimeric protein comprises: an extracellular domain of TNFR2 comprising an amino acid sequence that is at least about 90%, or at least about 95%, or at least about 97%, or at least about 98%, or at least about 99% identical to the amino acid sequence of SEQ ID NO: 62; a portion of DC-SIGN comprising an amino acid sequence that is at least about 90%, or at least about 95%, or at least about 97%, or at least about 98%, or at least about 99% identical to the amino acid sequence of SEQ ID NO: 65 or SEQ ID NO: 66; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, and SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain.

An illustrative TNFR2-Fc-DC-SIGN chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the extracellular domain of DC-SIGN is shown in an italics font):

(SEQ ID NO: 86)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDVVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDEPKSCDKTHTCPPCPAPEAAGGPSVFLFPPKPKDTL
MISRTPEVTCVVVDVSHEDPEVKFNWYVDGVEVHNAKTKPREEQYASTYRVVSVLTVLHQDWLNGKE
YKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGKIEGRMD
QVSKVPSSISQEQSRQDAIYQNLTQLKAAVGELSEKSKLQEIYQELTQLKAAVGELPEKSKLQEIYQELT
RLKAAVGELPEKSKLQEIYQELTWLKAAVGELPEKSKMQEIYQELTRLKAAVGELPEKSKQQEIYQELTR
LKAAVGELPEKSKQQEIYQELTRLKAAVGELPEKSKQQEIYQELTQLKAAVERLCHPCPWEWTFFQGN
CYFMSNSQRNWHDSITACKEVGAQLVVIKSAEEQNFLQLQSSRSNRFTWMGLSDLNQEGTWQWVDG
SPLLPSFKQYWNRGEPNNVGEEDCAEFSGNGWNDDKCNLAKFWICKKSAASCSRDEEQFLSPAPATP
NPPPA.

In embodiments, the chimeric protein comprises a variant of the TNFR2-Fc-DC-SIGN chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 86.

An illustrative TNFR2-Fc-DC-SIGN chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the C-type lectin binding domain (CLD) of DC-SIGN is shown in an italics font):

(SEQ ID NO: 87)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDVVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDEPKSCDKTHTCPPCPAPEAAGGPSVFLFPPKPKDTL
MISRTPEVTCVVVDVSHEDPEVKFNWYVDGVEVHNAKTKPREEQYASTYRVVSVLTVLHQDWLNGKE
YKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGKIEGRMD
HPCPWEWTFFQGNCYFMSNSQRNWHDSITACKEVGAQLVVIKSAEEQNFLQLQSSRSNRFTWMGLS
DLNQEGTWQWVDGSPLLPSFKQYWNRGEPNNVGEEDCAEFSGNGWNDDKCNLAKFWICK.

In embodiments, the chimeric protein comprises a variant of the TNFR2-Fc-DC-SIGN chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 87.

In embodiments, where the chimeric protein comprises an extracellular domain (ECD) of TNFR2, a joining linker preceding an Fc domain, the Fc domain, a joining linker following the Fc domain, and a portion of Dectin-2, the chimeric protein may comprise the following structure:

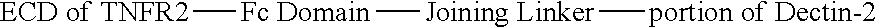

In embodiments, the chimeric protein comprises: an extracellular domain of TNFR2 comprising an amino acid sequence that is at least about 90%, or at least about 95%, or at least about 97%, or at least about 98%, or at least about 99% identical to the amino acid sequence of SEQ ID NO: 62; a portion of Dectin-2 comprising an amino acid sequence that is at least about 90%, or at least about 95%, or at least about 97%, or at least about 98%, or at least about 99% identical to the amino acid sequence of SEQ ID NO: 67 or SEQ ID NO: 68; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain.

An illustrative TNFR2-Fc-Dectin-2 chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the C-type lectin binding domain (CLD) of Dectin-2 is shown in an italics font):

(SEQ ID NO: 88)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDVVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDEPKSCDKTHTCPPCPAPEAAGGPSVFLFPPKPKDTL
MISRTPEVTCVVVDVSHEDPEVKFNWYVDGVEVHNAKTKPREEQYASTYRVVSVLTVLHQDWLNGKE
YKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGKIEGRMD
TYHFTYGETGKRLSELHSYHSSLTCFSEGTKVPAWGCCPASWKSFGSSCYFISSEEKVWSKSEQNCVE
MGAHLVVFNTEAEQNFIVQQLNESFSYFLGLSDPQGNNNWQWIDKTPYEKNVRFWHLGEPNHSAEQC
ASIVFWKPTGWGWNDVICETRRNSICEMNKIYL.

In embodiments, the chimeric protein comprises a variant of the TNFR2-Fc-Dectin-2 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 88.

In embodiments, where the chimeric protein comprises an extracellular domain (ECD) of TNFR2, a joining linker preceding an Fc domain, the Fc domain, a joining linker following the Fc domain, and a portion of TGF-beta, the chimeric protein may comprise the following structure:

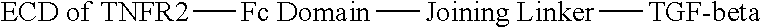

In embodiments, the chimeric protein comprises: an extracellular domain of TNFR2 comprising an amino acid sequence that is at least about 90%, or at least about 95%, or at least about 97%, or at least about 98%, or at least about 99% identical to the amino acid sequence of SEQ ID NO: 62; TGF-beta comprising an amino acid sequence that is at least about 90%, or at least about 95%, or at least about 97%, or at least about 98%, or at least about 99% identical to the amino acid sequence of SEQ ID NO: 72; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain.

An illustrative TNFR2-Fc-TGF-beta chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and TGF-beta is shown in an italics font):

(SEQ ID NO: 89)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDVVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDSKYGPPCPPCPAPEFLGGPSVFLFPPKPKDQLMISR
TPEVTCVVVDVSQEDPEVQFNWYVDGVEVHNAKTKPREEQFNSTYRVVSVLTVLHQDWLSGKEYKC
KVSSKGLPSSIEKTISNATGQPREPQVYTLPPSQEEMTKNQVSLTCLVKGFYPSDIAVEWESNGQPEN
NYKTTPPVLDSDGSFFLYSRLTVDKSRWQEGNVFSCSVLHEALHNHYTQKSLSLSLGKIEGRMDALDT
NYCFSSTEKNCCVRQLYIDFRKDLGWKWIHEPKGYHANFCLGPCPYIWSLDTQYSKVLALYNQHNPGA
SAAPCCVPQALEPLPIVYYVGRKPKVEQLSNMIVRSCKCS.

In embodiments, the chimeric protein comprises a variant of the TNFR2-Fc-TGF-beta chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 89.

In one aspect, the present disclosure relates to an isolated polynucleotide encoding a chimeric protein having a general structure of: N terminus—(a)-(b)-(c)—C terminus, wherein: (a) is a first domain comprising an extracellular domain of programmed death-1 (PD-1), or a variant or a fragment thereof that capable of binding a PD-1 ligand, (c) is a second domain comprising an extracellular domain of OX40 ligand (OX40L), or a variant or a fragment thereof that capable of binding an OX40L receptor, or 4-1BB Ligand (4-1BBL), or a variant or a fragment thereof that capable of binding a 4-1BBL receptor, (b) is a linker adjoining the first and second domains, wherein the linker comprises at least one cysteine residue capable of forming a disulfide bond and/or comprises a hinge-CH2-CH3 Fc domain. In embodiments, the PD-1 ligand is PD-L1 or PD-L2.

In embodiments, the OX40L receptor is OX40. In embodiments, the 4-1BBL receptor is 4-1BB. In embodiments, the isolated polynucleotide is or comprises an mRNA. In embodiments, the isolated polynucleotide is or comprises an mRNA that is modified according to any of the embodiments disclosed herein.

In embodiments, where the chimeric protein comprises an extracellular domain (ECD) of PD-1, a joining linker preceding an Fc domain, the Fc domain, a joining linker following the Fc domain, and a portion of OX40L, the chimeric protein may comprise the following structure:

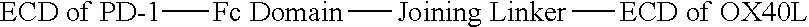

In embodiments, the chimeric protein comprises: an extracellular domain of PD-1 comprising an amino acid sequence that is at least about 90%, or at least about 95%, or at least about 97%, or at least about 98%, or at least about 99% identical to the amino acid sequence of SEQ ID NO: 73; a portion of OX40L comprising an amino acid sequence that is at least about 90%, or at least about 95%, or at least about 97%, or at least about 98%, or at least about 99% identical to the amino acid sequence of SEQ ID NO: 74; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain.

An illustrative PD-1-Fc-OX40L chimeric protein has the following sequence (the extracellular domain of PD-1 is shown by an underline, a linker comprising a mutant Fc domain of human IgG1 is shown in boldface font, with mutations shown by an underline, a joining linker is shown in an underlined-boldface-italic font, and OX40L is shown in an italics font):

(SEQ ID NO: 90)
LDSPDRPWNPPTFSPALLVVTEGDNATFTCSFSNTSESFVLNWYRMSPSNQTDKLAAFPEDRSQPGQ
DCRFRVTQLPNGRDFHMSVVRARRNDSGTYLCGAISLAPKAQIKESLRAELRVTERRAEVPTAHPSPSP
RPAGQFQSKYGPPCPSCPAPEFLGGPSVFLFPPKPKDTLMISRTPEVTCVVVDVSQEDPEVQFNWYV
DGVEVHNAKTKPREEQFNSTYRVVSVLTVLHQDWLSGKEYKCKVSSKGLPSSIEKTISNATGQPREP
QVYTLPPSQEEMTKNQVSLTCLVKGFYPSDIAVEWESNGQPENNYKTTPPVLDSDGSFFLYSRLTVDK
SSWQEGNVFSCSVMHEALHNHYTQKSLSLSLGKIEGRMDQVSHRYPRIQSIKVQFTEYKKEKGFILTS
QKEDEIMKVQNNSVIINCDGFYLISLKGYFSQEVNISLHYQKDEEPLFQLKKVRSVNSLMVASLTYKDKVY
LNVTTDNTSLDDFHVNGGELILIHQNPGEFCVL.

In embodiments, the chimeric protein comprises a variant of the PD-1-Fc-OX40L chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 90.

In embodiments, where the chimeric protein comprises an extracellular domain (ECD) of PD-1, a joining linker preceding an Fc domain, the Fc domain, a joining linker following the Fc domain, and a portion of 4-1BBL, the chimeric protein may comprise the following structure:

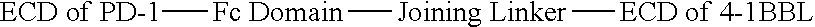

In embodiments, the chimeric protein comprises: an extracellular domain of PD-1 comprising an amino acid sequence that is at least about 90%, or at least about 95%, or at least about 97%, or at least about 98%, or at least about 99% identical to the amino acid sequence of SEQ ID NO: 73; a portion of 4-1BBL comprising an amino acid sequence that is at least about 90%, or at least about 95%, or at least about 97%, or at least about 98%, or at least about 99% identical to the amino acid sequence of SEQ ID NO: 75; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain.

An illustrative PD-1-Fc-4-1BBL chimeric protein has the following sequence (the extracellular domain of PD-1 is shown by an underline, a linker comprising a mutant Fc domain of human IgG1 is shown in boldface font, with mutations shown by an underline, a joining linker is shown in an underlined-boldface-italic font, and 4-1BBL is shown in an italics font):

(SEQ ID NO: 90)
LDSPDRPWNPPTFSPALLVVTEGDNATFTCSFSNTSESFVLNWYRMSPSNQTDKLAAFPEDRSQPGQ
DCRFRVTQLPNGRDFHMSVVRARRNDSGTYLCGAISLAPKAQIKESLRAELRVTERRAEVPTAHPSPSP
RPAGQFQSKYGPPCPSCPAPEFLGGPSVFLFPPKPKDTLMISRTPEVTCVVVDVSQEDPEVQFNWYV
DGVEVHNAKTKPREEQFNSTYRVVSVLTVLHQDWLSGKEYKCKVSSKGLPSSIEKTISNATGQPREP
QVYTLPPSQEEMTKNQVSLTCLVKGFYPSDIAVEWESNGQPENNYKTTPPVLDSDGSFFLYSRLTVDK
SSWQEGNVFSCSVMHEALHNHYTQKSLSLSLGKIEGRMDACPWAVSGARASPGSAASPRLREGPEL
SPDDPAGLLDLRQGMFAQLVAQNVLLIDGPLSWYSDPGLAGVSLTGGLSYKEDTKELVVAKAGVYYVF
FQLELRRVVAGEGSGSVSLALHLQPLRSAAGAAALALTVDLPPASSEARNSAFGFQGRLLHLSAGQRL
GVHLHTEARARHAWQLTQGATVLGLFRVTPEIPAGLPSPRSE.

In embodiments, the chimeric protein comprises a variant of the PD-1-Fc-4-1BBL chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 90.

In one aspect, the present disclosure relates to an isolated polynucleotide encoding a chimeric protein having a general structure of: N terminus—(a)-(b)-(c)—C terminus, wherein: (a) is a first domain comprising an extracellular domain of T-cell immunoglobulin mucin receptor 3 (TIM3), or a variant or a fragment thereof that capable of binding a TIM3 ligand, (c) is a second domain comprising an extracellular domain of CD40 ligand (CD40L), or a variant or a fragment thereof that capable of binding a CD40L receptor, (b) is a linker adjoining the first and second domains, wherein the linker comprises at least one cysteine residue capable of forming a disulfide bond and/or comprises a hinge-CH2-CH3 Fc domain. In embodiments, the TIM3 ligand is selected from galectin 9, phosphatidylserine (PtdSer), CEACAM1 and high mobility group protein B1 (HMGB1). In embodiments, the CD40L receptor is CD40. In embodiments, the isolated polynucleotide is or comprises an mRNA. In embodiments, the isolated polynucleotide is or comprises an mRNA that is modified according to any of the embodiments disclosed herein.

In embodiments, where the chimeric protein comprises an extracellular domain (ECD) of TIM-3, a joining linker preceding an Fc domain, the Fc domain, a joining linker following the Fc domain, and a portion of CD40L, the chimeric protein may comprise the following structure:

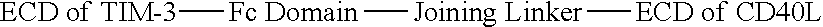

In embodiments, the chimeric protein comprises: an extracellular domain of TIM-3 comprising an amino acid sequence that is at least about 90%, or at least about 95%, or at least about 97%, or at least about 98%, or at least about 99% identical to the amino acid sequence of SEQ ID NO: 76; a portion of CD40L comprising an amino acid sequence that is at least about 90%, or at least about 95%, or at least about 97%, or at least about 98%, or at least about 99% identical to the amino acid sequence of SEQ ID NO: 77; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain.

An illustrative TIM-3-Fc-CD40L chimeric protein has the following sequence (the extracellular domain of TIM-3 is shown by an underline, a linker comprising a mutant Fc domain of human IgG1 is shown in boldface font, with mutations shown by an underline, a joining linker is shown in an underlined-boldface-italic font, and CD40L is shown in an italics font):

(SEQ ID NO: 91)
SEVEYRAEVGQNAYLPCFYTPAAPGNLVPVCWGKGACPVFECGNWVLRTDERDVNYWTSRYWLNGDFR
KGDVSLTIENVTLADSGIYCCRIQIPGIMNDEKFNLKLVIKPAKVTPAPTRQRDFTAAFPRMLTTRGHGPAET
QTLGSLPDINLTQISTLANELRDSRLANDLRDSGATIRIGSKYGPPCPPCPAPEFLGGPSVFLFPPKPKDQL
MISRTPEVTCVVVDVSQEDPEVQFNWYVDGVEVHNAKTKPREEQFNSTYRVVSVLTVLHQDWLSGKEYK
CKVSSKGLPSSIEKTISNATGQPREPQVYTLPPSQEEMTKNQVSLTCLVKGFYPSDIAVEWESNGQPENN
YKTTPPVLDSDGSFFLYSRLTVDKSRWQEGNVFSCSVLHEALHNHYTQKSLSLSLGKIEGRMDHRRLDKI
EDERNLHEDFVFMKTIQRCNTGERSLSLLNCEEIKSQFEGFVKDIMLNKEETKKENSFEMQKGDQNPQIAA
HVISEASSKTTSVLQWAEKGYYTMSNNLVTLENGKQLTVKRQGLYYIYAQVTFCSNREASSQAPFIASLCLK
SPGRFERILLRAANTHSSAKPCGQQSIHLGGVFELQPGASVFVNVTDPSQVSHGTGFTSFGLLKL.

In embodiments, a chimeric protein comprises a variant of a TIM-3-Fc-CD40L chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 91.

In one aspect, the present disclosure relates to an isolated polynucleotide encoding a chimeric protein having a general structure of: N terminus—(a)-(b)-(c)—C terminus, wherein: (a) is a first domain comprising an extracellular domain of V-set and immunoglobulin domain-containing protein 8 (VSIG8), or a variant or a fragment thereof that capable of binding a VSIG8 ligand, (c) is a second domain comprising an extracellular domain of 4-1BB Ligand (4-1BBL), or a variant or a fragment thereof that capable of binding a 4-1BBL receptor, (b) is a linker adjoining the first and second domains, wherein the linker comprises at least one cysteine residue capable of forming a disulfide bond and/or comprises a hinge-CH2-CH3 Fc domain. In embodiments, the 4-1BBL receptor is 4-1BB. In embodiments, the isolated polynucleotide is or comprises an mRNA. In embodiments, the isolated polynucleotide is or comprises an mRNA that is modified according to any of the embodiments disclosed herein.

In embodiments, where the chimeric protein comprises an extracellular domain (ECD) of VSIG8, a joining linker preceding an Fc domain, the Fc domain, a joining linker following the Fc domain, and a portion of 4-1BBL, the chimeric protein may comprise the following structure:

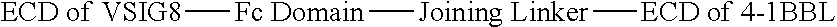

In embodiments, the chimeric protein comprises: an extracellular domain of VSIG8 comprising an amino acid sequence that is at least about 90%, or at least about 95%, or at least about 97%, or at least about 98%, or at least about 99% identical to the amino acid sequence of SEQ ID NO: 78; a portion of 4-1BBL comprising an amino acid sequence that is at least about 90%, or at least about 95%, or at least about 97%, or at least about 98%, or at least about 99% identical to the amino acid sequence of SEQ ID NO: 75; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain.

An illustrative VSIG8-Fc-4-1BBL chimeric protein has the following sequence (the extracellular domain of VSIG8 is shown by an underline, a linker comprising a mutant Fc domain of human IgG1 is shown in boldface font, with mutations shown by an underline, a joining linker is shown in an underlined-boldface-italic font, and 4-1BBL is shown in an italics font):

(SEQ ID NO: 92)
VRINGDGQEVLYLAEGDNVRLGCPYVLDPEDYGPNGLDIEWMQVNSDPAHHRENVFLSYQDKRINHGSLP
HLQQRVRFAASDPSQYDASINLMNLQVSDTATYECRVKKTTMATRKVIVTVQARPAVPMCWTEGHMTYG
NDVVLKCYASGGSQPLSYKWAKISGHHYPYRAGSYTSQHSYHSELSYQESFHSSINQGLNNGDLVLKDIS
RADDGLYQCTVANNVGYSVCVVEVKVSDSRRIGSKYGPPCPPCPAPEFLGGPSVFLFPPKPKDQLMISRT
PEVTCVVVDVSQEDPEVQFNWYVDGVEVHNAKTKPREEQFNSTYRVVSVLTVLHQDWLSGKEYKCKVS
SKGLPSSIEKTISNATGQPREPQVYTLPPSQEEMTKNQVSLTCLVKGFYPSDIAVEWESNGQPENNYKTT
PPVLDSDGSFFLYSRLTVDKSRWQEGNVFSCSVLHEALHNHYTQKSLSLSLGKIEGRMDACPWAVSGAR
ASPGSAASPRLREGPELSPDDPAGLLDLRQGMFAQLVAQNVLLIDGPLSWYSDPGLAGVSLTGGLSYKED
TKELVVAKAGVYYVFFQLELRRVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPSPRSE.

In embodiments, a chimeric protein comprises a variant of a VSIG8-Fc-4-1BBL chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 92.

In one aspect, the present disclosure relates to an isolated polynucleotide encoding a chimeric protein having a general structure of: N terminus—(a)-(b)-(c)—C terminus, wherein: (a) is a first domain comprising an extracellular domain of signal regulatory protein a (SIRPα), or a variant or a fragment thereof that is capable of binding a SIRPα ligand, (c) is a second domain comprising an extracellular domain of CD40 ligand (CD40L), or a variant or a fragment thereof that is capable of binding a CD40L receptor, (b) is a linker adjoining the first and second domains, wherein the linker comprises at least one cysteine residue capable of forming a disulfide bond and/or comprises a hinge-CH2-CH3 Fc domain. In embodiments, the SIRPα ligand is CD47. In embodiments, the CD40L receptor is CD40. In embodiments, the isolated polynucleotide is or comprises an mRNA. In embodiments, the isolated polynucleotide is or comprises an mRNA that is modified according to any of the embodiments disclosed herein.

In embodiments, where the chimeric protein comprises an extracellular domain (ECD) of SIRPα, a joining linker preceding an Fc domain, the Fc domain, a joining linker following the Fc domain, and a portion of CD40L, the chimeric protein may comprise the following structure:

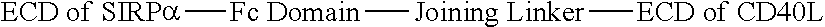

In embodiments, the chimeric protein comprises: an extracellular domain of SIRPα comprising an amino acid sequence that is at least about 90%, or at least about 95%, or at least about 97%, or at least about 98%, or at least about 99% identical to the amino acid sequence of SEQ ID NO: 79; a portion of CD40L comprising an amino acid sequence that is at least about 90%, or at least about 95%, or at least about 97%, or at least about 98%, or at least about 99% identical to the amino acid sequence of SEQ ID NO: 77; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain.

An illustrative SIRPα-Fc-CD40L chimeric protein has the following sequence (the extracellular domain of SIRPα is shown by an underline, a linker comprising a mutant Fc domain of human IgG1 is shown in boldface font, with mutations shown by an underline, a joining linker is shown in an underlined-boldface-italic font, and CD40L is shown in an italics font):

(SEQ ID NO: 93)
EEELQVIQPDKSVLVAAGETATLRCTATSLIPVGPIQWFRGAGPGRELIYNQKEGHFPRVTTVSDLTKRNNM
DFSIRIGNITPADAGTYYCVKFRKGSPDDVEFKSGAGTELSVRAKPSAPVVSGPAARATPQHTVSFTCESH
GFSPRDITLKWFKNGNELSDFQTNVDPVGESVSYSIHSTAKVVLTREDVHSQVICEVAHVTLQGDPLRGTA
NLSETIRVPPTLEVTQQPVRAENQVNVTCQVRKFYPQRLQLTWLENGNVSRTETASTVTENKDGTYNWMS
WLLVNVSAHRDDVKLTCQVEHDGQPAVSKSHDLKVSAHPKEQGSNTAAENTGSNERNIYGSKYGPPCPP
CPAPEFLGGPSVFLFPPKPKDQLMISRTPEVTCVVVDVSQEDPEVQFNWYVDGVEVHNAKTKPREEQFN
STYRVVSVLTVLHQDWLSGKEYKCKVSSKGLPSSIEKTISNATGQPREPQVYTLPPSQEEMTKNQVSLTC
LVKGFYPSDIAVEWESNGQPENNYKTTPPVLDSDGSFFLYSRLTVDKSRWQEGNVFSCSVLHEALHNHY
TQKSLSLSLGKIEGRMDHRRLDKIEDERNLHEDFVFMKTIQRCNTGERSLSLLNCEEIKSQFEGFVKDIMLN
KEETKKENSFEMQKGDQNPQIAAHVISEASSKTTSVLQWAEKGYYTMSNNLVTLENGKQLTVKRQGLYYIY
AQVTFCSNREASSQAPFIASLCLKSPGRFERILLRAANTHSSAKPCGQQSIHLGGVFELQPGASVFVNVTD
PSQVSHGTGFTSFGLLKL.

In embodiments, a chimeric protein comprises a variant of a SIRPα-Fc-CD40L chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 93.

### Isolated Polynucleotides

In one aspect, the present disclosure provides an isolated polynucleotide encoding the chimeric protein of any of the embodiments disclosed herein.

In one aspect, the present disclosure relates to a method for inducing lymphocyte margination in a human subject in need thereof, the method comprising a step of administering to the subject a pharmaceutical composition comprising an isolated polynucleotide encoding a chimeric protein having a general structure of: N terminus—(a)-(b)-(c)—C terminus wherein: (a) a first domain comprising a portion of a Type I transmembrane protein at or near the N-terminus; (c) a second domain comprising a portion of a Type II transmembrane protein at or near the C-terminus; and (b) is a linker adjoining the first and second domains, wherein the linker comprises at least one cysteine residue capable of forming a disulfide bond and/or comprises a hinge-CH2-CH3 Fc domain, wherein the first domain is an immune inhibitory signal, and the second domain is an immune stimulatory signal, wherein the isolated polynucleotide is the isolated polynucleotide of any of the embodiments disclosed herein.

In one aspect, the present disclosure relates to an isolated polynucleotide encoding a chimeric protein having a general structure of: N terminus—(a)-(b)-(c)—C terminus, wherein: (a) is a first domain comprising an extracellular domain of T cell immunoreceptor with Ig and ITIM domains (TIGIT), or a variant or a fragment thereof that is capable of binding a TIGIT ligand, (c) is a second domain comprising an extracellular domain of glucocorticoid-induced TNFR-related protein ligand (GITRL), or a variant or a fragment thereof that is capable of binding a GITRL receptor, or LIGHT (homologous to lymphotoxin, exhibits inducible expression and competes with HSV glycoprotein D for binding to herpesvirus entry mediator, a receptor expressed on T lymphocytes), or a variant or a fragment thereof that is capable of binding a LIGHT receptor, (b) is a linker adjoining the first and second domains, wherein the linker comprises at least one cysteine residue capable of forming a disulfide bond and/or comprises a hinge-CH2-CH3 Fc domain. In embodiments, the TIGIT ligand is selected from CD155/PVR, Nectin-2, Nectin-3 and Nectin-4. In embodiments, the GITRL receptor is glucocorticoid-induced TNFR-related protein (GITR). In embodiments, the LIGHT receptor is TNFRSF3/LTBR. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein that comprises a portion of TIGIT, a joining linker preceding an Fc domain, the Fc domain, a joining linker following the Fc domain, and a portion GITRL, and the chimeric protein may comprise the following structure:

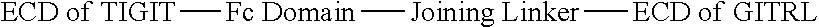

In one aspect, the present disclosure relates to an isolated polynucleotide encoding a chimeric protein that: an extracellular domain of TIGIT, capable of binding a TIGIT ligand, and comprising an amino acid sequence that is at least about 90%, or at least about 95%, or at least about 97%, or at least about 98%, or at least about 99% identical to the amino acid sequence of SEQ ID NO: 59; an extracellular domain of GITRL, capable of binding a GITRL receptor, and comprising an amino acid sequence that is at least about 90%, or at least about 95%, or at least about 97%, or at least about 98%, or at least about 99% identical to the amino acid sequence of SEQ ID NO: 60; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a TIGIT-Fc-GITRL chimeric protein has the following sequence (the extracellular domain (ECD) of human TIGIT is indicated by underline, a variant IgG4 CH2-CH3-Fc domain is shown in an italic font, joining linkers are shown in a boldface font, and the extracellular domain (ECD) of human GITRL is indicated by an underlined, italic font):

(SEQ ID NO: 80)
MMTGTIETTGNISAEKGGSIILQCHLSSTTAQVTQVNWEQQDQLLAICNADLGWHISPSFKDRVAPGPGLGL
TLQSLTVNDTGEYFCIYHTYPDGTYTGRIFLEVLESSVAEHGARFQIPSKYGPPCPPCPAPEFLGGPSVFLF
PPKPKDQLMISRTPEVTCVVVDVSQEDPEVQFNWYVDGVEVHNAKTKPREEQFNSTYRVVSVLTVLHQD
WLSGKEYKCKVSSKGLPSSIEKTISNATGQPREPQVYTLPPSQEEMTKNQVSLTCLVKGFYPSDIAVEWES
NGQPENNYKTTPPVLDSDGSFFLYSRLTVDKSRWQEGNVFSCSVLHEALHNHYTQKSLSLSLGKIEGRMD
ETAKEPCMAKFGPLPSKWQMASSEPPCVNKVSDWKLEILQNGLYLIYGQVAPNANYNDVAPFEVRLYKNK
DMIQTLTNKSKIQNVGGTYELHVGDTIDLIFNSEHQVLKNNTYWGIILLANPQFIS.

In one aspect, the present disclosure relates to an isolated polynucleotide encoding a chimeric protein that comprises a variant of the TIGIT-Fc-GITRL chimeric protein. In embodiments the variant has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 80. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein that comprises a portion of TIGIT, a joining linker preceding an Fc domain, the Fc domain, a joining linker following the Fc domain, and a portion LIGHT, and the chimeric protein may comprise the following structure:

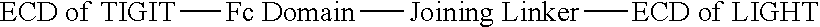

In one aspect, the present disclosure relates to an isolated polynucleotide encoding a chimeric protein that: an extracellular domain of TIGIT, capable of binding a TIGIT ligand, and comprising an amino acid sequence that is at least about 90%, or at least about 95%, or at least about 97%, or at least about 98%, or at least about 99% identical to the amino acid sequence of SEQ ID NO: 59; an extracellular domain of LIGHT, capable of binding a LIGHT receptor, and comprising an amino acid sequence that is at least about 90%, or at least about 95%, or at least about 97%, or at least about 98%, or at least about 99% identical to the amino acid sequence of SEQ ID NO: 61; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a TIGIT-Fc-LIGHT chimeric protein has the following sequence (the extracellular domain of TIGIT is shown by an underline, a linker comprising a mutant Fc domain of human IgG1 is shown in boldface font, with mutations shown by an underline, a joining linker is shown in an underlined-boldface-italic font, and the extracellular domain of LIGHT is shown in an italics font):

(SEQ ID NO: 81)
MMTGTIETTGNISAEKGGSIILQCHLSSTTAQVTQVNWEQQDQLLAICNADLGWHISPSFKDRVAPGPGLGL
TLQSLTVNDTGEYFCIYHTYPDGTYTGRIFLEVLESSVAEHGARFQIPSKYGPPCPPCPAPEFLGGPSVFLF
PPKPKDQLMISRTPEVTCVVVDVSQEDPEVQFNWYVDGVEVHNAKTKPREEQFNSTYRVVSVLTVLHQD
WLSGKEYKCKVSSKGLPSSIEKTISNATGQPREPQVYTLPPSQEEMTKNQVSLTCLVKGFYPSDIAVEWES
NGQPENNYKTTPPVLDSDGSFFLYSRLTVDKSRWQEGNVFSCSVLHEALHNHYTQKSLSLSLGKIEGRMD
LQLHWRLGEMVTRLPDGPAGSWEQLIQERRSHEVNPAAHLTGANSSLTGSGGPLLWETQLGLAFLRGLS
YHDGALVVTKAGYYYIYSKVQLGGVGCPLGLASTITHGLYKRTPRYPEELELLVSQQSPCGRATSSSRVW
WDSSFLGGVVHLEAGEKVVVRVLDERLVRLRDGTRSYFGAFMV.

In one aspect, the present disclosure relates to an isolated polynucleotide encoding a chimeric protein that comprises a variant of the TIGIT-Fc-LIGHT chimeric protein. In embodiments the variant has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 81. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein having a general structure of: N terminus—(a)-(b)-(c)—C terminus, wherein: (a) is a first domain comprising an extracellular domain of tumor necrosis factor (TNF) receptor 2 (TNFR2), or a variant or a fragment thereof that is capable of binding a TNFR2 ligand, (c) is a second domain comprising an extracellular domain selected from CLEC7a, or a variant or a fragment thereof that capable of binding a CLEC7a ligand, DC-SIGN(CD209), or a variant or a fragment thereof that capable of binding a DC-SIGN(CD209) ligand, DECTIN2(CLEC6A), or a variant or a fragment thereof that capable of binding a DECTIN2(CLEC6A) ligand, Langerin(CD207,CLC4K), or a variant or a fragment thereof that capable of binding a Langerin(CD207,CLC4K) ligand, CD69, or a variant or a fragment thereof that capable of binding a CD69 ligand, and TGF-beta, or a variant or a fragment thereof that capable of binding a TGF-beta receptor, (b) is a linker adjoining the first and second domains, wherein the linker comprises at least one cysteine residue capable of forming a disulfide bond and/or comprises a hinge-CH2-CH3 Fc domain. In embodiments, the TNFR2 ligand is TNFα. In embodiments, the CLEC7a ligand is a beta-1,3-linked and/or beta-1,6-linked glucan. In embodiments, the DC-SIGN(CD209) ligand is a Intercellular Adhesion Molecule 2 (ICAM2) and/or Intercellular Adhesion Molecule 3 (ICAM3). In embodiments, the DECTIN2(CLEC6A) ligand is an alpha-mannan. In embodiments, the Langerin(CD207,CLC4K) ligand is a sulfated glycan, a mannosylated glycan, a keratan sulfate (KS) and/or a beta-glucan. In embodiments, the CD69 ligand is Galectin-1 (Gal-1) or the S100A8/S100A9 complex. In embodiments, the TGF-beta receptor is TGFBR1 and TGFBR2. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein that comprises a portion of TNFR2, a joining linker preceding an Fc domain, the Fc domain, a joining linker following the Fc domain, and a portion Clec7a, the chimeric protein may comprise the following structure:

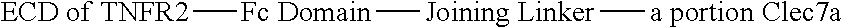

In one aspect, the present disclosure relates to an isolated polynucleotide encoding a chimeric protein that: an extracellular domain of TNFR2 comprising an amino acid sequence that is at least about 90%, or at least about 95%, or at least about 97%, or at least about 98%, or at least about 99% identical to the amino acid sequence of SEQ ID NO: 62; a portion of Clec7a capable of binding a beta-1,3-linked and/or beta-1,6-linked glucan, and comprising an amino acid sequence that is at least about 90%, or at least about 95%, or at least about 97%, or at least about 98%, or at least about 99% identical to the amino acid sequence of SEQ ID NO: 63 or SEQ ID NO: 64; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a TNFR2-Fc-Clec7a chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the extracellular domain of Clec7a is shown in an italics font):

(SEQ ID NO: 82)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDVVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDEPKSCDKTHTCPPCPAPEAAGGPSVFLFPPKPKDTL
MISRTPEVTCVVVDVSHEDPEVKFNWYVDGVEVHNAKTKPREEQYASTYRVVSVLTVLHQDWLNGKE
YKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGKIEGRMD
TMAIWRSNSGSNTLENGYELSRNKENHSQPTQSSLEDSVTPTKAVKTTGVLSSPCPPNWIIYEKSCYLF
SMSLNSWDGSKRQCWQLGSNLLKIDSSNELGFIVKQVSSQPDNSFWIGLSRPQTEVPWLWEDGSTFS
SNLFQIRTTATQENPSPNCVWIHVSVIYDQLCSVPSYSICEKKFSM.

In one aspect, the present disclosure relates to an isolated polynucleotide encoding a chimeric protein that comprises a variant of the TNFR2-Fc-Clec7a chimeric protein. In embodiments the variant has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 82. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a TNFR2-Fc-Clec7a chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the C-type lectin binding domain (CLD) of Clec7a is shown in an italics font):

(SEQ ID NO: 83)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDVVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDEPKSCDKTHTCPPCPAPEAAGGPSVFLFPPKPKDTL
MISRTPEVTCVVVDVSHEDPEVKFNWYVDGVEVHNAKTKPREEQYASTYRVVSVLTVLHQDWLNGKE
YKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGKIEGRMD
SSPCPPNWIIYEKSCYLFSMSLNSWDGSKRQCWQLGSNLLKIDSSNELGFIVKQVSSQPDNSFWIGLSR
PQTEVPWLWEDGSTFSSNLFQIRTTATQENPSPNCVWIHVSVIYDQLCSVPSYSICEKKFSM.

In one aspect, the present disclosure relates to an isolated polynucleotide encoding a chimeric protein that comprises a variant of the TNFR2-Fc-Clec7a chimeric protein. In embodiments the variant has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 83. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein that comprises an extracellular domain (ECD) of TNFR2, a joining linker preceding an Fc domain, the Fc domain, a joining linker following the Fc domain, and a portion of langerin, the chimeric protein may comprise the following structure:

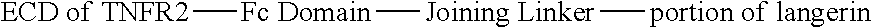

In one aspect, the present disclosure relates to an isolated polynucleotide encoding a chimeric protein that: an extracellular domain of TNFR2 comprising an amino acid sequence that is at least about 90%, or at least about 95%, or at least about 97%, or at least about 98%, or at least about 99% identical to the amino acid sequence of SEQ ID NO: 62; a portion of langerin capable of binding a ligand comprising a sulfated glycan, a mannosylated glycan, a keratan sulfate (KS) and/or a beta-glucan, and comprising an amino acid sequence that is at least about 90%, or at least about 95%, or at least about 97%, or at least about 98%, or at least about 99% identical to the amino acid sequence of SEQ ID NO: 69 or SEQ ID NO: 70; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3 or SEQ ID NO: 74. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a TNFR2-Fc-langerin chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the extracellular domain of langerin is shown in an italics font):

(SEQ ID NO: 84)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDWVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDEPKSCDKTHTCPPCPAPEAAGGPSVFLFPPKPKDTL
MISRTPEVTCVVVDVSHEDPEVKFNWYVDGVEVHNAKTKPREEQYASTYRVVSVLTVLHQDWLNGKE
YKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGKIEGRMD
PRFMGTISDVKTNVQLLKGRVDNISTLDSEIKKNSDGMEAAGVQIQMVNESLGYVRSQFLKLKTSVEKA
NAQIQILTRSWEEVSTLNAQIPELKSDLEKASALNTKIRALQGSLENMSKLLKRQNDILQVVSQGWKYFK
GNFYYFSLIPKTWYSAEQFCVSRNSHLTSVTSESEQEFLYKTAGGLIYWIGLTKAGMEGDWSWVDDTP
FNKVQSVRFWIPGEPNNAGNNEHCGNIKAPSLQAWNDAPCDKTFLFICKRPYVPSEP.

In one aspect, the present disclosure relates to an isolated polynucleotide encoding a chimeric protein that comprises a variant of the TNFR2-Fc-langerin chimeric protein. In embodiments the variant has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 84. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a TNFR2-Fc-langerin chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the C-type lectin binding domain (CLD) of langerin is shown in an italics font):

(SEQ ID NO: 85)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDVVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDEPKSCDKTHTCPPCPAPEAAGGPSVFLFPPKPKDTL
MISRTPEVTCVVVDVSHEDPEVKFNWYVDGVEVHNAKTKPREEQYASTYRVVSVLTVLHQDWLNGKE
YKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGKIEGRMD
QVVSQGWKYFKGNFYYFSLIPKTWYSAEQFCVSRNSHLTSVTSESEQEFLYKTAGGLIYWIGLTKAGME
GDWSWVDDTPFNKVQSVRFWIPGEPNNAGNNEHCGNIKAPSLQAWNDAPCDKTFLFICKRPYVPSEP.

In one aspect, the present disclosure relates to an isolated polynucleotide encoding a chimeric protein that comprises a variant of the TNFR2-Fc-langerin chimeric protein. In embodiments the variant has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 85. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein that comprises an extracellular domain (ECD) of TNFR2, a joining linker preceding an Fc domain, the Fc domain, a joining linker following the Fc domain, and a portion of DC-SIGN the chimeric protein may comprise the following structure:

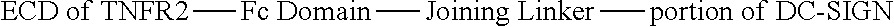

In one aspect, the present disclosure relates to an isolated polynucleotide encoding a chimeric protein that: an extracellular domain of TNFR2 comprising an amino acid sequence that is at least about 90%, or at least about 95%, or at least about 97%, or at least about 98%, or at least about 99% identical to the amino acid sequence of SEQ ID NO: 62; a portion of DC-SIGN comprising an amino acid sequence that is at least about 90%, or at least about 95%, or at least about 97%, or at least about 98%, or at least about 99% identical to the amino acid sequence of SEQ ID NO: 65 or SEQ ID NO: 66; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, and SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a TNFR2-Fc-DC-SIGN chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the extracellular domain of DC-SIGN is shown in an italics font):

(SEQ ID NO: 86)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDWVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDEPKSCDKTHTCPPCPAPEAAGGPSVFLFPPKPKDTL
MISRTPEVTCVVVDVSHEDPEVKFNWYVDGVEVHNAKTKPREEQYASTYRVVSVLTVLHQDWLNGKE
YKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGKIEGRMD
QVSKVPSSISQEQSRQDAIYQNLTQLKAAVGELSEKSKLQEIYQELTQLKAAVGELPEKSKLQEIYQELT
RLKAAVGELPEKSKLQEIYQELTWLKAAVGELPEKSKMQEIYQELTRLKAAVGELPEKSKQQEIYQELTR
LKAAVGELPEKSKQQEIYQELTRLKAAVGELPEKSKQQEIYQELTQLKAAVERLCHPCPWEWTFFQGN
CYFMSNSQRNWHDSITACKEVGAQLVVIKSAEEQNFLQLQSSRSNRFTWMGLSDLNQEGTWQWVDG
SPLLPSFKQYWNRGEPNNVGEEDCAEFSGNGWNDDKCNLAKFWICKKSAASCSRDEEQFLSPAPATP
NPPPA.

In one aspect, the present disclosure relates to an isolated polynucleotide encoding a chimeric protein that comprises a variant of the TNFR2-Fc-DC-SIGN chimeric protein. In embodiments the variant has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 86. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a TNFR2-Fc-DC-SIGN chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the C-type lectin binding domain (CLD) of DC-SIGN is shown in an italics font):

(SEQ ID NO: 87)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDVVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDEPKSCDKTHTCPPCPAPEAAGGPSVFLFPPKPKDTL
MISRTPEVTCVVVDVSHEDPEVKFNWYVDGVEVHNAKTKPREEQYASTYRVVSVLTVLHQDWLNGKE
YKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGKIEGRMD
HPCPWEWTFFQGNCYFMSNSQRNWHDSITACKEVGAQLVVIKSAEEQNFLQLQSSRSNRFTWMGLS
DLNQEGTWQWVDGSPLLPSFKQYWNRGEPNNVGEEDCAEFSGNGWNDDKCNLAKFWICK.

In one aspect, the present disclosure relates to an isolated polynucleotide encoding a chimeric protein that comprises a variant of the TNFR2-Fc-DC-SIGN chimeric protein. In embodiments the variant has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 87. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein that comprises an extracellular domain (ECD) of TNFR2, a joining linker preceding an Fc domain, the Fc domain, a joining linker following the Fc domain, and a portion of Dectin-2, the chimeric protein may comprise the following structure:

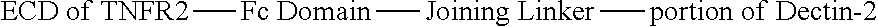

In one aspect, the present disclosure relates to an isolated polynucleotide encoding a chimeric protein that: an extracellular domain of TNFR2 comprising an amino acid sequence that is at least about 90%, or at least about 95%, or at least about 97%, or at least about 98%, or at least about 99% identical to the amino acid sequence of SEQ ID NO: 62; a portion of Dectin-2 comprising an amino acid sequence that is at least about 90%, or at least about 95%, or at least about 97%, or at least about 98%, or at least about 99% identical to the amino acid sequence of SEQ ID NO: 67 or SEQ ID NO: 68; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a TNFR2-Fc-Dectin-2 chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the C-type lectin binding domain (CLD) of Dectin-2 is shown in an italics font):

(SEQ ID NO: 88)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDVVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDEPKSCDKTHTCPPCPAPEAAGGPSVFLFPPKPKDTL
MISRTPEVTCVVVDVSHEDPEVKFNWYVDGVEVHNAKTKPREEQYASTYRVVSVLTVLHQDWLNGKE
YKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGKIEGRMD
TYHFTYGETGKRLSELHSYHSSLTCFSEGTKVPAWGCCPASWKSFGSSCYFISSEEKVWSKSEQNCVE
MGAHLVVENTEAEQNFIVQQLNESFSYFLGLSDPQGNNNWQWIDKTPYEKNVRFWHLGEPNHSAEQC
ASIVFWKPTGWGWNDVICETRRNSICEMNKIYL.

In one aspect, the present disclosure relates to an isolated polynucleotide encoding a chimeric protein that comprises a variant of the TNFR2-Fc-Dectin-2 chimeric protein. In embodiments the variant has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 88. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein that comprises an extracellular domain (ECD) of TNFR2, a joining linker preceding an Fc domain, the Fc domain, a joining linker following the Fc domain, and a portion of TGF-beta, the chimeric protein may comprise the following structure:

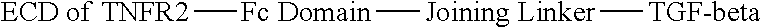

In one aspect, the present disclosure relates to an isolated polynucleotide encoding a chimeric protein that: an extracellular domain of TNFR2 comprising an amino acid sequence that is at least about 90%, or at least about 95%, or at least about 97%, or at least about 98%, or at least about 99% identical to the amino acid sequence of SEQ ID NO: 62; TGF-beta comprising an amino acid sequence that is at least about 90%, or at least about 95%, or at least about 97%, or at least about 98%, or at least about 99% identical to the amino acid sequence of SEQ ID NO: 72; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a TNFR2-Fc-TGF-beta chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and TGF-beta is shown in an italics font):

(SEQ ID NO: 89)
LPAQVAFTPYAPEPGSTCRLREYYDQTAQMCCSKCSPGQHAKVFCTKTSDTVCDSCEDSTYTQLWNW
VPECLSCGSRCSSDQVETQACTREQNRICTCRPGWYCALSKQEGCRLCAPLRKCRPGFGVARPGTET
SDVVCKPCAPGTFSNTTSSTDICRPHQICNVVAIPGNASMDAVCTSTSPTRSMAPGAVHLPQPVSTRSQ
HTQPTPEPSTAPSTSFLLPMGPSPPAEGSTGDSKYGPPCPPCPAPEFLGGPSVFLFPPKPKDQLMISR
TPEVTCVVVDVSQEDPEVQFNWYVDGVEVHNAKTKPREEQFNSTYRVVSVLTVLHQDWLSGKEYKC
KVSSKGLPSSIEKTISNATGQPREPQVYTLPPSQEEMTKNQVSLTCLVKGFYPSDIAVEWESNGQPEN
NYKTTPPVLDSDGSFFLYSRLTVDKSRWQEGNVFSCSVLHEALHNHYTQKSLSLSLGKIEGRMDALDT
NYCFSSTEKNCCVRQLYIDFRKDLGWKWIHEPKGYHANFCLGPCPYIWSLDTQYSKVLALYNQHNPGA
SAAPCCVPQALEPLPIVYYVGRKPKVEQLSNMIVRSCKCS.

In one aspect, the present disclosure relates to an isolated polynucleotide encoding a chimeric protein that comprises a variant of the TNFR2-Fc-TGF-beta chimeric protein. In embodiments the variant has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 89. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein having a general structure of: N terminus—(a)-(b)-(c)—C terminus, wherein: (a) is a first domain comprising an extracellular domain of programmed death-1 (PD-1), or a variant or a fragment thereof that capable of binding a PD-1 ligand, (c) is a second domain comprising an extracellular domain of OX40 ligand (OX40L), or a variant or a fragment thereof that capable of binding an OX40L receptor, or 4-1BB Ligand (4-1BBL), or a variant or a fragment thereof that capable of binding a 4-1BBL receptor, (b) is a linker adjoining the first and second domains, wherein the linker comprises at least one cysteine residue capable of forming a disulfide bond and/or comprises a hinge-CH2-CH3 Fc domain. In embodiments, the PD-1 ligand is PD-L1 or PD-L2. In embodiments, the OX40L receptor is OX40. In embodiments, the 4-1BBL receptor is 4-1BB. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein that comprises an extracellular domain (ECD) of PD-1, a joining linker preceding an Fc domain, the Fc domain, a joining linker following the Fc domain, and a portion of OX40L, the chimeric protein may comprise the following structure:

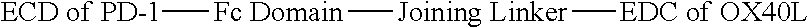

In one aspect, the present disclosure relates to an isolated polynucleotide encoding a chimeric protein that: an extracellular domain of PD-1 comprising an amino acid sequence that is at least about 90%, or at least about 95%, or at least about 97%, or at least about 98%, or at least about 99% identical to the amino acid sequence of SEQ ID NO: 73; a portion of OX40L comprising an amino acid sequence that is at least about 90%, or at least about 95%, or at least about 97%, or at least about 98%, or at least about 99% identical to the amino acid sequence of SEQ ID NO: 74; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a PD-1-Fc-OX40L chimeric protein has the following sequence (the extracellular domain of PD-1 is shown by an underline, a linker comprising a mutant Fc domain of human IgG1 is shown in boldface font, with mutations shown by an underline, a joining linker is shown in an underlined-boldface-italic font, and OX40L is shown in an italics font):

(SEQ ID NO: 90)
LDSPDRPWNPPTFSPALLVVTEGDNATFTCSFSNTSESFVLNWYRMSPSNQTDKLAAFPEDRSQPGQ
DCRFRVTQLPNGRDFHMSVVRARRNDSGTYLCGAISLAPKAQIKESLRAELRVTERRAEVPTAHPSPSP
RPAGQFQSKYGPPCPSCPAPEFLGGPSVFLFPPKPKDTLMISRTPEVTCVVVDVSQEDPEVQFNWYV
DGVEVHNAKTKPREEQFNSTYRVVSVLTVLHQDWLSGKEYKCKVSSKGLPSSIEKTISNATGQPREP
QVYTLPPSQEEMTKNQVSLTCLVKGFYPSDIAVEWESNGQPENNYKTTPPVLDSDGSFFLYSRLTVDK
SSWQEGNVFSCSVMHEALHNHYTQKSLSLSLGKIEGRMDQVSHRYPRIQSIKVQFTEYKKEKGFILTS
QKEDEIMKVQNNSVIINCDGFYLISLKGYFSQEVNISLHYQKDEEPLFQLKKVRSVNSLMVASLTYKDKVY
LNVTTDNTSLDDFHVNGGELILIHQNPGEFCVL.

In one aspect, the present disclosure relates to an isolated polynucleotide encoding a chimeric protein that comprises a variant of the PD-1-Fc-OX40L chimeric protein. In embodiments the variant has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 90. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein that comprises an extracellular domain (ECD) of PD-1, a joining linker preceding an Fc domain, the Fc domain, a joining linker following the Fc domain, and a portion of 4-1BBL, the chimeric protein may comprise the following structure:

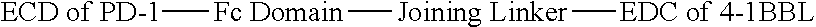

In one aspect, the present disclosure relates to an isolated polynucleotide encoding a chimeric protein that: an extracellular domain of PD-1 comprising an amino acid sequence that is at least about 90%, or at least about 95%, or at least about 97%, or at least about 98%, or at least about 99% identical to the amino acid sequence of SEQ ID NO: 73; a portion of 4-1BBL comprising an amino acid sequence that is at least about 90%, or at least about 95%, or at least about 97%, or at least about 98%, or at least about 99% identical to the amino acid sequence of SEQ ID NO: 75; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a PD-1-Fc-4-1BBL chimeric protein has the following sequence (the extracellular domain of PD-1 is shown by an underline, a linker comprising a mutant Fc domain of human IgG1 is shown in boldface font, with mutations shown by an underline, a joining linker is shown in an underlined-boldface-italic font, and 4-1BBL is shown in an italics font):

(SEQ ID NO: 90)
LDSPDRPWNPPTFSPALLVVTEGDNATFTCSFSNTSESFVLNWYRMSPSNQTDKLAAFPEDRSQPGQ
DCRFRVTQLPNGRDFHMSVVRARRNDSGTYLCGAISLAPKAQIKESLRAELRVTERRAEVPTAHPSPSP
RPAGQFQSKYGPPCPSCPAPEFLGGPSVFLFPPKPKDTLMISRTPEVTCVVVDVSQEDPEVQFNWYV
DGVEVHNAKTKPREEQFNSTYRVVSVLTVLHQDWLSGKEYKCKVSSKGLPSSIEKTISNATGQPREP
QVYTLPPSQEEMTKNQVSLTCLVKGFYPSDIAVEWESNGQPENNYKTTPPVLDSDGSFFLYSRLTVDK
SSWQEGNVFSCSVMHEALHNHYTQKSLSLSLGKIEGRMDACPWAVSGARASPGSAASPRLREGPEL
SPDDPAGLLDLRQGMFAQLVAQNVLLIDGPLSWYSDPGLAGVSLTGGLSYKEDTKELVVAKAGVYYVF
FQLELRRVVAGEGSGSVSLALHLQPLRSAAGAAALALTVDLPPASSEARNSAFGFQGRLLHLSAGQRL
GVHLHTEARARHAWQLTQGATVLGLFRVTPEIPAGLPSPRSE.

In one aspect, the present disclosure relates to an isolated polynucleotide encoding a chimeric protein that comprises a variant of the PD-1-Fc-4-1BBL chimeric protein. In embodiments the variant has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 90. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein having a general structure of: N terminus—(a)-(b)-(c)—C terminus, wherein: (a) is a first domain comprising an extracellular domain of T-cell immunoglobulin mucin receptor 3 (TIM3), or a variant or a fragment thereof that capable of binding a TIM3 ligand, (c) is a second domain comprising an extracellular domain of CD40 ligand (CD40L), or a variant or a fragment thereof that capable of binding a CD40L receptor, (b) is a linker adjoining the first and second domains, wherein the linker comprises at least one cysteine residue capable of forming a disulfide bond and/or comprises a hinge-CH2-CH3 Fc domain. In embodiments, the TIM3 ligand is selected from galectin 9, phosphatidylserine (PtdSer), CEACAM1 and high mobility group protein B1 (HMGB1). In embodiments, the CD40L receptor is CD40. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein that comprises an extracellular domain (ECD) of TIM-3, a joining linker preceding an Fc domain, the Fc domain, a joining linker following the Fc domain, and a portion of CD40L, the chimeric protein may comprise the following structure:

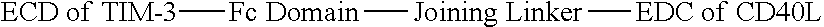

In one aspect, the present disclosure relates to an isolated polynucleotide encoding a chimeric protein that: an extracellular domain of TIM-3 comprising an amino acid sequence that is at least about 90%, or at least about 95%, or at least about 97%, or at least about 98%, or at least about 99% identical to the amino acid sequence of SEQ ID NO: 76; a portion of CD40L comprising an amino acid sequence that is at least about 90%, or at least about 95%, or at least about 97%, or at least about 98%, or at least about 99% identical to the amino acid sequence of SEQ ID NO: 77; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a TIM-3-Fc-CD40L chimeric protein has the following sequence (the extracellular domain of TIM-3 is shown by an underline, a linker comprising a mutant Fc domain of human IgG1 is shown in boldface font, with mutations shown by an underline, a joining linker is shown in an underlined-boldface-italic font, and CD40L is shown in an italics font):

(SEQ ID NO: 91)
SEVEYRAEVGQNAYLPCFYTPAAPGNLVPVCWGKGACPVFECGNVVLRT
DERDVNYWTSRYWLNGDFRKGDVSLTIENVTLADSGIYCCRIQIPGIMN
DEKFNLKLVIKPAKVTPAPTRQRDFTAAFPRMLTTRGHGPAETQTLGSL
PDINLTQISTLANELRDSRLANDLRDSGATIRIGSKYGPPCPPCPAPEF
LGGPSVFLFPPKPKDQLMISRTPEVTCVVVDVSQEDPEVQFNWYVDGVE
VHNAKTKPREEQFNSTYRVVSVLTVLHQDWLSGKEYKCKVSSKGLPSSI
EKTISNATGQPREPQVYTLPPSQEEMTKNQVSLTCLVKGFYPSDIAVEW
ESNGQPENNYKTTPPVLDSDGSFFLYSRLTVDKSRWQEGNVFSCSVLHE
ALHNHYTQKSLSLSLGKIEGRMDHRRLDKIEDERNLHEDFVFMKTIQRC
NTGERSLSLLNCEEIKSQFEGFVKDIMLNKEETKKENSFEMQKGDQNPQ
IAAHVISEASSKTTSVLQWAEKGYYTMSNNLVTLENGKQLTVKRQGLYY
IYAQVTFCSNREASSQAPFIASLCLKSPGRFERILLRAANTHSSAKPCG
QQSIHLGGVFELQPGASVFVNVTDPSQVSHGTGFTSFGLLKL.

In embodiments, the isolated polynucleotide encodes a variant of a TIM-3-Fc-CD40L chimeric protein. In embodiments the variant has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 91. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein having a general structure of: N terminus—(a)-(b)-(c)—C terminus, wherein: (a) is a first domain comprising an extracellular domain of V-set and immunoglobulin domain-containing protein 8 (VSIG8), or a variant or a fragment thereof that capable of binding a VSIG8 ligand, (c) is a second domain comprising an extracellular domain of 4-1BB Ligand (4-1BBL), or a variant or a fragment thereof that capable of binding a 4-1BBL receptor, (b) is a linker adjoining the first and second domains, wherein the linker comprises at least one cysteine residue capable of forming a disulfide bond and/or comprises a hinge-CH2-CH3 Fc domain. In embodiments, the 4-1BBL receptor is 4-1BB. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein that comprises an extracellular domain (ECD) of VSIG8, a joining linker preceding an Fc domain, the Fc domain, a joining linker following the Fc domain, and a portion of 4-1BBL, the chimeric protein may comprise the following structure:

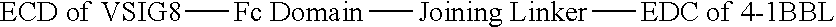

In one aspect, the present disclosure relates to an isolated polynucleotide encoding a chimeric protein that: an extracellular domain of VSIG8 comprising an amino acid sequence that is at least about 90%, or at least about 95%, or at least about 97%, or at least about 98%, or at least about 99% identical to the amino acid sequence of SEQ ID NO: 78; a portion of 4-1BBL comprising an amino acid sequence that is at least about 90%, or at least about 95%, or at least about 97%, or at least about 98%, or at least about 99% identical to the amino acid sequence of SEQ ID NO: 75; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a VSIG8-Fc-4-1BBL chimeric protein has the following sequence (the extracellular domain of VSIG8 is shown by an underline, a linker comprising a mutant Fc domain of human IgG1 is shown in boldface font, with mutations shown by an underline, a joining linker is shown in an underlined-boldface-italic font, and 4-1BBL is shown in an italics font):

(SEQ ID NO: 92)
VRINGDGQEVLYLAEGDNVRLGCPYVLDPEDYGPNGLDIEWMQVNSDPAHHRENVFLSYQDKRINHGSLP
HLQQRVRFAASDPSQYDASINLMNLQVSDTATYECRVKKTTMATRKVIVTVQARPAVPMCWTEGHMTYG
NDWVLKCYASGGSQPLSYKWAKISGHHYPYRAGSYTSQHSYHSELSYQESFHSSINQGLNNGDLVLKDIS
RADDGLYQCTVANNVGYSVCWVEVKVSDSRRIGSKYGPPCPPCPAPEFLGGPSVFLFPPKPKDQLMISRT
PEVTCVVVDVSQEDPEVQFNWYVDGVEVHNAKTKPREEQFNSTYRVVSVLTVLHQDWLSGKEYKCKVS
SKGLPSSIEKTISNATGQPREPQVYTLPPSQEEMTKNQVSLTCLVKGFYPSDIAVEWESNGQPENNYKTT
PPVLDSDGSFFLYSRLTVDKSRWQEGNVFSCSVLHEALHNHYTQKSLSLSLGKIEGRMDACPWAVSGAR
ASPGSAASPRLREGPELSPDDPAGLLDLRQGMFAQLVAQNVLLIDGPLSWYSDPGLAGVSLTGGLSYKED
TKELVVAKAGVYYVFFQLELRRVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPSPRSE.

In embodiments, the isolated polynucleotide encodes a variant of a VSIG8-Fc-4-1BBL chimeric protein. In embodiments the variant has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 92. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein having a general structure of: N terminus—(a)-(b)-(c)—C terminus, wherein: (a) is a first domain comprising an extracellular domain of signal regulatory protein a (SIRPα), or a variant or a fragment thereof that is capable of binding a SIRPα ligand, (c) is a second domain comprising an extracellular domain of CD40 ligand (CD40L), or a variant or a fragment thereof that is capable of binding a CD40L receptor, (b) is a linker adjoining the first and second domains, wherein the linker comprises at least one cysteine residue capable of forming a disulfide bond and/or comprises a hinge-CH2-CH3 Fc domain. In embodiments, the SIRPα ligand is CD47. In embodiments, the CD40L receptor is CD40. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a chimeric protein that comprises an extracellular domain (ECD) of SIRPα, a joining linker preceding an Fc domain, the Fc domain, a joining linker following the Fc domain, and a portion of CD40L, the chimeric protein may comprise the following structure:

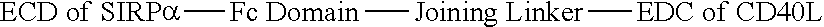

In one aspect, the present disclosure relates to an isolated polynucleotide encoding a chimeric protein that: an extracellular domain of SIRPα comprising an amino acid sequence that is at least about 90%, or at least about 95%, or at least about 97%, or at least about 98%, or at least about 99% identical to the amino acid sequence of SEQ ID NO: 79; a portion of CD40L comprising an amino acid sequence that is at least about 90%, or at least about 95%, or at least about 97%, or at least about 98%, or at least about 99% identical to the amino acid sequence of SEQ ID NO: 77; and a linker adjoining the extracellular domains. In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5. In embodiments, the linker further comprises the linker comprises one or more joining linkers, such joining linkers independently selected from SEQ ID NOs: 6 to 52. In embodiments, the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to an isolated polynucleotide encoding a SIRPα-Fc-CD40L chimeric protein has the following sequence (the extracellular domain of SIRPα is shown by an underline, a linker comprising a mutant Fc domain of human IgG1 is shown in boldface font, with mutations shown by an underline, a joining linker is shown in an underlined-boldface-italic font, and CD40L is shown in an italics font):

(SEQ ID NO: 93)
EEELQVIQPDKSVLVAAGETATLRCTATSLIPVGPIQWFRGAGPGRELIYNQKEGHFPRVTTVSDLTKRNNM
DFSIRIGNITPADAGTYYCVKFRKGSPDDVEFKSGAGTELSVRAKPSAPVVSGPAARATPQHTVSFTCESH
GFSPRDITLKWFKNGNELSDFQTNVDPVGESVSYSIHSTAKVVLTREDVHSQVICEVAHVTLQGDPLRGTA
NLSETIRVPPTLEVTQQPVRAENQVNVTCQVRKFYPQRLQLTWLENGNVSRTETASTVTENKDGTYNWMS
WLLVNVSAHRDDVKLTCQVEHDGQPAVSKSHDLKVSAHPKEQGSNTAAENTGSNERNIYGSKYGPPCPP
CPAPEFLGGPSVFLFPPKPKDQLMISRTPEVTCVVVDVSQEDPEVQFNWYVDGVEVHNAKTKPREEQFN
STYRVVSVLTVLHQDWLSGKEYKCKVSSKGLPSSIEKTISNATGQPREPQVYTLPPSQEEMTKNQVSLTC
LVKGFYPSDIAVEWESNGQPENNYKTTPPVLDSDGSFFLYSRLTVDKSRWQEGNVFSCSVLHEALHNHY
TQKSLSLSLGKIEGRMDHRRLDKIEDERNLHEDFVFMKTIQRCNTGERSLSLLNCEEIKSQFEGFVKDIMLN
KEETKKENSFEMQKGDQNPQIAAHVISEASSKTTSVLQWAEKGYYTMSNNLVTLENGKQLTVKRQGLYYIY
AQVTFCSNREASSQAPFIASLCLKSPGRFERILLRAANTHSSAKPCGQQSIHLGGVFELQPGASVFVNVTD
PSQVSHGTGFTSFGLLKL.

In embodiments, the isolated polynucleotide encodes a variant of a SIRPα-Fc-CD40L chimeric protein. In embodiments the variant has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 93. In embodiments, the isolated polynucleotide is or comprises an mRNA. In embodiments, the isolated polynucleotide is or comprises an mRNA that is modified according to any of the embodiments disclosed herein.

In one aspect, the present disclosure provides a host cell comprising the vector of any of the embodiments disclosed herein. In one aspect, the present disclosure provides a host cell comprising an RNA (without limitations, e.g., mmRNA) encoring the chimeric protein of any of the embodiments disclosed herein. A host cell comprising the nucleic acid, e.g., the mmRNA of any of the embodiments disclosed herein.

In embodiments, the polynucleotide is RNA, optionally, an mRNA. In embodiments, the polynucleotide is codon optimized.

In embodiments, the polynucleotide is or comprises an mRNA or a modified mRNA (mmRNA). In embodiments, the polypeptide may include a polynucleotide modification including, but not limited to, a nucleoside modification. In embodiments, the polynucleotide is or comprises an mmRNA. In embodiments, the mmRNA comprises one or more nucleoside modifications. In embodiments, the nucleoside modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof.

In embodiments, the polypeptide the at least one chemically modified nucleoside is selected from pseudouridine (Ψ), N1-methylpseudouridine (m1Ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1Ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methylinosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and two or more combinations thereof.

In embodiments, the mmRNA does not cause a substantial induction of the innate immune response of a cell into which the mmRNA is introduced. In embodiments, the modification in the mmRNA enhance one or more of the efficiency of production of the chimeric protein, intracellular retention of the mmRNA, and viability of contacted cells, and possess reduced immunogenicity.

In embodiments, the mmRNA has a length sufficient to include an open reading frame encoding the chimeric protein of the present disclosure.

Modified mRNAs need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of a nucleic acid such that the function of the nucleic acid is not substantially decreased. A modification may also be a 5′ or 3′ terminal modification. The nucleic acids may contain at a minimum one and at maximum 100% modified nucleotides, or any intervening percentage, such as at least about 50% modified nucleotides, at least about 80% modified nucleotides, or at least about 90% modified nucleotides.

In embodiments, the mmRNA may contain a modified pyrimidine such as uracil or cytosine. In embodiments, at least about 5%, at least about 10%, at least about 25%, at least about 50%, In embodiments, the modified uracil may be replaced by a compound having a single unique structure, or can be replaced by a plurality of compounds having different structures disclosed above (e.g., same mmRNA may contain 2, 3, 4 or more types of uniquely modified uracil). In embodiments, at least about 5%, at least about 10%, at least about 25%, at least about 50%, at least about 80%, at least about 90% or 100% of the cytosine in the nucleic acid may be replaced with a modified cytosine. The modified cytosine can be replaced by a compound having a single unique structure, or can be replaced by a plurality of compounds having different structures disclosed above (e.g., same mmRNA may contain 2, 3, 4 or more types of uniquely modified cytosine).

In embodiments, the mmRNA comprises at least one chemically modified nucleoside. In embodiments, wherein the at least one chemically modified nucleoside is selected from pseudouridine (Ψ), N1-methylpseudouridine (m1Ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1Ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methylinosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and two or more combinations thereof. In embodiments, the mmRNA comprises at least one chemically modified nucleoside, wherein the at least one chemically modified nucleoside is selected from pseudouridine, N1-methylpseudouridine, 5-methylcytosine, 5-methoxyuridine, and a combination thereof. In embodiments, the mmRNA comprises at least one chemically modified nucleoside is N1-methylpseudouridine. In embodiments, the mmRNA is fully modified with chemically-modified uridines. In embodiments, the mmRNA is a fully modified N1-methylpseudouridine mRNA. Additional chemical modifications are disclosed in US Patent Application Publication No. 20190111003, the entire contents of which are hereby incorporated by reference

In embodiments, modified nucleosides include pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In embodiments, modified nucleosides include 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In embodiments, the nucleotide can be modified on the major groove face and can include replacing hydrogen on C-5 of uracil with a methyl group or a halo group.

In embodiments, a modified nucleoside is 5′-O-(1-Thiophosphate)-Adenosine, 5′-O-(1-Thiophosphate)-Cytidine, 5′-O-(1-Thiophosphate)-Guanosine, 5′-O-(1-Thiophosphate)-Uridine or 5′-O-(1-Thiophosphate)-Pseudouridine.

Further examples of modified nucleotides and modified nucleotide combinations are disclosed in U.S. Pat. Nos. 8,710,200; 8,822,663; 8,999,380; 9,181,319; 9,254,311; 9,334,328; 9,464,124; 9,950,068; 10,626,400; 10,808,242; 11,020,477, US Patent Application Publication Nos. 20220001026, 20210318817, 20210283262, 20200360481, 20200113844, 20200085758, 20170204152, 20190114089, 20190114090, 20180369374, 20180318385, 20190111003, and PCT International Application Publication Nos. WO/2017112943, WO 2014/028429, WO 2017/201325 the entire contents of which are hereby incorporated by reference. The methods for synthesizing the modified mRNA are disclosed, e.g., in US Patent Application Publication Nos. 20170204152, the entire contents of which are hereby incorporated by reference.

In embodiments, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the cytosine residues of the mmRNA are replaced by a modified cytosine residues. In embodiments, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the uracil residues of the mmRNA are replaced by a modified uracil residues.

In embodiments, the mmRNA further comprises a 5′ untranslated region (UTR) and/or a 3′ UTR, wherein either or both may independently contain one or more different nucleoside modifications. In such embodiments, nucleoside modifications may also be present in the translatable region. In embodiments, the mmRNA further comprises a Kozak sequence. In embodiments, the mmRNA further comprises a internal ribosome entry site (IRES).

In embodiments, the mmRNA further comprises a 5′-cap and/or a poly A tail.

In embodiments, the 5′-cap contains a 5′-5′-triphosphate linkage between the 5′-most nucleotide and guanine nucleotide. In embodiments, the 5′-cap comprises a methylation of the ultimate and penultimate most 5′-nucleotides on the 2′-hydroxyl group. In embodiments, the 5′-cap facilitates binding the mRNA Cap Binding Protein (CBP), confers mRNA stability in the cell and/or confers translation competency.

In embodiments, the poly-A tail is greater than about 30 nucleotides, or greater than about 40 nucleotides in length. In embodiments, the poly-A tail at least about 40 nucleotides, or at least about 45 nucleotides, or at least about 55 nucleotides, or at least about 60 nucleotides, or at least about 80 nucleotides, or at least about 90 nucleotides, or at least about 100 nucleotides, or at least about 120 nucleotides, or at least about 140 nucleotides, or at least about 160 nucleotides, or at least about 180 nucleotides, or at least about 200 nucleotides, or at least about 250 nucleotides, or at least about 300 nucleotides, or at least about 350 nucleotides, or at least about 400 nucleotides, or at least about 450 nucleotides, or at least about 500 nucleotides, or at least about 600 nucleotides, or at least about 700 nucleotides, or at least about 800 nucleotides, or at least about 900 nucleotides, or at least about 1000 nucleotides in length.

In embodiments, the mmRNA comprises a 3′ untranslated region (UTR). In embodiments, the 3′ UTR comprises a nucleic acid sequence at least 90%, 95%, 96%, 97%, 98%, 99%, or 100% identical to a sequence listed in Table 4 of US Patent Application Publication No. 20190114089, which is incorporated herein in its entirety. In embodiments, the 3′ UTR comprises at least one microRNA-122 (miR-122) binding site, wherein the miR-122 binding site is a miR-122-3p binding site or a miR-122-5-binding site. In embodiments, the mmRNA comprises a nucleic acid sequence comprising a miRNA binding site. In some embodiments, the miRNA binding site binds to miR-122. In a particular embodiment, the miRNA binding site binds to miR-122-3p or miR-122-5p. In embodiments, the mmRNA comprises at least two, at least three, at least four, at least five, at least six, at least seven, at least eight, at least nine, or at least ten miRNA binding sites.

In embodiments, the miRNA binding site is inserted within the 3′ UTR. In embodiments, the polynucleotide further comprises a spacer sequence between the open reading frame and the miRNA binding site. In one aspect, the spacer sequence comprises at least about 10 nucleotides, at least about 20 nucleotides, at least about 30 nucleotides, at least about 40 nucleotides, at least about 50 nucleotides, at least about 60 nucleotides, at least about 70 nucleotides, at least about 80 nucleotides, at least about 90 nucleotides, or at least about 100 nucleotides.

In embodiments, the mmRNA further comprises a 5′ UTR. In embodiments, the 5′ UTR comprises a nucleic acid sequence at least 90%, 95%, 96%, 97%, 98%, 99%, or 100% identical to a sequence listed in Table 3 of US Patent Application Publication No. 20190114089, or a sequence disclosed in PCT International Application Publication Nos. WO 2017/201325 and WO 2014/164253, each of which is incorporated herein in its entirety. In embodiments, the 5′ UTR bears features, which play roles in translation initiation. In embodiments, the 5′ UTR harbors signatures like Kozak sequences which are commonly known to be involved in the process by which the ribosome initiates translation of many genes. In embodiments, the 5′ UTR forms secondary structures which are involved in elongation factor binding. In embodiments, the 5′ UTR of mRNA known to be upregulated in cancers, such as c-myc, may be used to enhance expression of a nucleic acid molecule, such as a polynucleotides, in cancer cells. In embodiments, the 5′ UTR of mRNA known to be upregulated in liver and/or spleen may be used to enhance expression of a nucleic acid molecule, such as a polynucleotides, in liver and/or spleen.

In embodiments, at least one of the regions of linked nucleosides of A comprises a sequence of linked nucleosides which functions as a 5′ UTR and at least one of the regions of linked nucleosides of C comprises a sequence of linked nucleosides which functions as a 3′ UTR. In embodiments, the 5′ UTR and the 3′ UTR are from the same or different species. In embodiments, the 5′ UTR and the 3′ UTR may be the native untranslated regions from different proteins from the same or different species. In embodiments, the 5′ UTR and the 3′ UTR may have synthetic sequences.

In embodiments, the mmRNA further comprises a 3′ polyadenylation (polyA tail).

In embodiments, the mmRNA further comprises a 5′ terminal cap. In embodiments, the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

In embodiments, the polynucleotide is in vitro transcribed (IVT). In embodiments, the polynucleotide is chimeric. In embodiments, the polynucleotide is circular.

In embodiments, the mmRNAs of the present disclosure are produced by means available in the art, including but not limited to in vitro transcription (IVT) and synthetic methods. Enzymatic IVT, solid-phase, liquid-phase, combined synthetic methods, small region synthesis, and ligation methods may be utilized. In embodiments, mmRNAs are made using IVT enzymatic synthesis methods. Methods of making polynucleotides by IVT are known in the art and are described in International Application PCT International Patent Publication No. WO2013151666, the contents of which are incorporated herein by reference in their entirety. Accordingly, the present disclosure also includes polynucleotides, e.g., DNA, constructs and vectors that may be used to in vitro transcribe an mRNA described herein.

In embodiments, the polynucleotide is or comprises DNA. In embodiments, the polynucleotide is or comprises a minicircle or a plasmid DNA. In embodiments, the plasmid DNA is devoid of any prokaryotic components. In embodiments, the polynucleotide comprises a tissue-specific control element. In embodiments, the tissue-specific control element is a promoter or an enhancer. In embodiments, the plasmid DNA is an expression vector. In embodiments, the DNA is or comprises a minicircle. In embodiments, the minicircle is a circular molecule, which is optionally small. In embodiments, the minicircle utilizes a cellular transcription and translation machinery to produce an encoded gene product. In embodiments, the minicircle is devoid of any prokaryotic components. In embodiments, the minicircle only comprises substantially only sequences of mammalian origin (or those that have been optimized for mammalian cells). In embodiments, the minicircle lacks or has reduced amount of DNA sequence elements that are recognized by the innate immune system and/or toll-like receptors. In embodiments, the minicircle is produced by excising any bacterial components of from a parental plasmid, thereby making it smaller than a parental DNA sequence. In embodiments, the minicircle is of non-viral origin. In embodiments, the minicircle remains episomal. In embodiments, the minicircle does not replicate with a host cell. In embodiments, expression of the chimeric protein in non-dividing cells harboring a minicircle lasts for at least 2 days, or at least 4 days, or at least 6 days, or at least 8 days, or at least 10 days, or at least 12 days, or at least 14 days, or at least 16 days, or at least 18 days, or at least 20 days, or at least 22 days, or at least 24 days, or longer in dividing cells. In embodiments, expression of the chimeric protein in non-dividing cells harboring a minicircle lasts for at least 4 days, or at least 6 days, or at least 8 days, or at least 10 days, or at least 1 week, or at least 2 weeks, or at least 3 weeks, or at least 4 weeks, or at least 5 weeks, or at least 6 weeks, or at least 1 month, or at least 2 months, or at least 3 months, or at least 4 months, or at least 5 months, or at least 6 months, or at least 8 months, or longer in dividing cells.

In one aspect, the present disclosure provides a vector comprising the polynucleotide of any one of the embodiments disclosed herein. In embodiments, the chimeric protein can be provided as an expression vector. In embodiments, the expression vector is a DNA expression vector or an RNA expression vector. In embodiments, the expression vector is a viral expression vector. In embodiments, the expression vector is a non-viral expression vector (without limitation, e.g., a plasmid).

In embodiments, the present non-viral vectors are linear or circular DNA molecules that comprise a polynucleotide encoding a polypeptide and is operably linked to control sequences, wherein the control sequences provide for expression of the polynucleotide encoding the polypeptide. In embodiments, the non-viral vector comprises a promoter sequence, and transcriptional and translational stop signal sequences. In embodiments, the expression vector may include, among others, chromosomal and episomal vectors, e.g., vectors derived from bacterial plasmids, from transposons, from yeast episomes, from insertion elements, from yeast chromosomal elements, and vectors derived from combinations thereof. The present constructs may contain control regions that regulate as well as engender expression.

A vector generally comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In embodiments, the expression vector is an autonomously replicating plasmid or a virus (e.g., AAV vectors). In embodiments, the expression vector is non-plasmid and non-viral compounds that facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

In embodiments, the polynucleotide or cell therapy may employ expression vectors, which comprise the isolated polynucleotide encoding the chimeric protein operably linked to an expression control region that is functional in the host cell. The expression control region is capable of driving expression of the operably linked encoding nucleic acid such that the chimeric protein is produced in a human cell transformed with the expression vector. Expression control regions are regulatory polynucleotides (sometimes referred to herein as elements), such as promoters and enhancers, that influence expression of an operably linked nucleic acid. An expression control region of an expression vector is capable of expressing operably linked encoding nucleic acid in a human cell. In an embodiment, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. In various embodiments, the chimeric protein expression is inducible or repressible. Typically, the amount of increase or decrease conferred by such elements is proportional to the amount of signal present; the greater the amount of signal, the greater the increase or decrease in expression.

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription-initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), FIp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family, and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences including CRISPR/CAS9, zinc finger, TALEN, and meganuclease gene-editing technologies.

### Pharmaceutical Compositions

In one aspect, the present disclosure provides a pharmaceutical composition comprising a pharmaceutically acceptable excipient or carrier, and the chimeric protein of any of the embodiments disclosed herein, the isolated polynucleotide of any of the embodiments disclosed herein, the mmRNA of any of the embodiments disclosed herein, or the vector of any of the embodiments disclosed herein. In embodiments, the pharmaceutical composition comprises the mmRNA of any of the embodiments disclosed herein.

Suitable pharmaceutical compositions are disclosed in U.S. Pat. Nos. 8,710,200; 8,822,663; 8,999,380; 9,181,319; 9,254,311; 9,334,328; 9,464,124; 9,950,068; 10,626,400; 10,808,242; 11,020,477, US Patent Application Publication Nos. 20220001026, 20210318817, 20210283262, 20200360481, 20200113844, 20200085758, 20170204152, 20190114089, 20190114090, 20180369374, 20180318385, 20190111003, and PCT International Application Publication Nos. WO/2017112943, WO 2014/028429, WO 2017/201325 the entire contents of which are hereby incorporated by reference.

In one aspect, the present disclosure relates to a pharmaceutical composition comprising an isolated modified mRNA (mmRNA) encoding a heterologous chimeric protein having an amino acid sequence that has at least about 95% sequence identity with an amino acid sequence selected from SEQ ID NOs: 80-93.

In embodiments, the mmRNA comprises a modification (e.g., an RNA element), wherein the modification provides a desired translational regulatory activity. Such modifications are described in PCT Application No. PCT International Application Publication No. WO2018213789, the entire contents of which are herein incorporated by reference.

In embodiments, the mmRNA further comprises a 3′ untranslated region (UTR). In embodiments, the 3′ UTR comprises at least one microRNA-122 (miR-122) binding site. In embodiments, the miR-122 binding site is a miR-122-3p binding site or a miR-122-5-binding site. In embodiments, the mmRNA further comprises a spacer sequence between the open reading frame and the miRNA binding site. In embodiments, the spacer sequence comprises at least about 10 nucleotides, at least about 20 nucleotides, at least about 30 nucleotides, at least about 40 nucleotides, at least about 50 nucleotides, at least about 60 nucleotides, at least about 70 nucleotides, at least about 80 nucleotides, at least about 90 nucleotides, or at least about 100 nucleotides.

In embodiments, the mmRNA further comprises a 5′ UTR. In embodiments, the 5′ UTR harbors a Kozak sequence and/or forms a secondary structure that stimulate elongation factor binding.

In embodiments, the mmRNA further comprises a 5′ terminal cap. In embodiments, the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

In any of the embodiments disclosed herein, the mmRNA may comprise one or more modifications. In any of the embodiments disclosed herein, the mmRNA may comprise at least one modification. In embodiments, the modification is nucleoside modification. In embodiments, the modification is a base modification. In embodiments, the modification is a sugar-phosphate backbone modification.

In embodiments, the modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and a combination of any two or more thereof. In embodiments, the modifications are selected from pseudouridine (Ψ), N1-methylpseudouridine (m1Ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1Ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methylinosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and a combination of any two or more thereof. In embodiments, modification is selected from pseudouridine, N1-methylpseudouridine, 5-methylcytosine, 5-methoxyuridine, and a combination thereof.

In embodiments, the mmRNA comprises at least one N1-methylpseudouridine. In embodiments, the mmRNA is fully modified with chemically-modified uridines. In embodiments, the mmRNA is a fully modified with N1-methylpseudouridine.

In embodiments, the modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine or a combination of any two or more thereof.

In embodiments, the modifications are selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In embodiments, the modifications are selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In embodiments, the modifications are selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In embodiments, the modifications are present on the major groove face. In embodiments, a hydrogen on C-5 of uracil is replaced with a methyl group or a halo group.

In embodiments, the mmRNA further comprises one or more modifications selected from 5′-O-(1-Thiophosphate)-Adenosine, 5′-O-(1-Thiophosphate)-Cytidine, 5′-O-(1-Thiophosphate)-Guanosine, 5′-O-(1-Thiophosphate)-Uridine and 5′-O-(1-Thiophosphate)-Pseudouridine.

In any of the embodiments disclosed herein, the pharmaceutical composition may further comprise a lipidoid, a liposome, a lipoplex, a lipid nanoparticle, a polymeric nanoparticle, a peptide, a protein, a cell, a nanoparticle mimic, a nanotube, or a conjugate. In embodiments, the pharmaceutical composition is formulated as a lipid nanoparticle (LNP), a lipoplex, or a liposome. In embodiments, the pharmaceutical composition is formulated as a lipid nanoparticle (LNP). In embodiments, the mmRNAs described herein may be formulated in a cationic oil-in-water emulsion where the emulsion particle comprises an oil core and a cationic lipid that can interact with the mRNA anchoring the molecule to the emulsion particle. In embodiments, the mRNAs described herein may be formulated in a water-in-oil emulsion comprising a continuous hydrophobic phase in which the hydrophilic phase is dispersed. Exemplary emulsions can be made by the methods described in PCT International Application Publication Nos. WO2012006380 and WO201087791, each of which is herein incorporated by reference in its entirety.

In some embodiments, nucleic acids of the invention (e.g., mRNA) are formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the invention can be generated using components, compositions, and methods as are disclosed, e.g., in PCT International Application Publication Nos. WO2021231854, WO2021050986, WO2021055833, WO2021213924, WO2021055849, WO2021214204, WO2021188969, WO2021055835, WO2020061284, WO2020061295, WO2017049245, WO2017031232, WO2017112865, WO2017218704, WO2017218704, WO2017099823, WO2017049074, WO2017117528, WO2017180917, WO2017075531, WO2017223135, WO2016118724, WO2015164674, WO2015038892, WO2014152211, and WO2013090648, the entire contents of each which are herein incorporated by reference. PEG-lipids are known in the art, such as those described in U.S. Pat. No. 8,158,601 and PCT International Application Publication Nos. WO2012099755 and WO 2015/130584, which are incorporated herein by reference in their entirety. The ionizable lipid may be selected from, but not limited to, a ionizable lipid described in International Publication Nos. WO2013086354 and WO2013116126; the contents of each of which are herein incorporated by reference in their entirety. In embodiments, the lipid may be a cleavable lipid such as those described in PCT International Publication No. WO2012170889, herein incorporated by reference in its entirety. In embodiments, the lipid may be synthesized by methods known in the art and/or as described in International Publication Nos. WO2013086354; the contents of each of which are herein incorporated by reference in their entirety. In embodiments, the LNP formulations described herein can additionally comprise a permeability enhancer molecule. Non-limiting permeability enhancer molecules are described in U.S. Publication No. US20050222064, herein incorporated by reference in its entirety.

In embodiments, the pharmaceutical composition is formulated as a lipid nanoparticle (LNP). In embodiments, the LNP comprises a molar ratio of about 20-60% ionizable amino lipid, about 5-25% phospholipid, about 25-55% structural lipid, and about 0.5-1.5% PEG lipid. In embodiments, the LNP comprises a molar ratio of about 50% ionizable amino lipid, about 8-12% phospholipid, about 37-40% structural lipid, and about 1-2% PEG lipid. In embodiments, the lipid nanoparticles comprise lipids selected from an ionizable lipid (e.g., an ionizable cationic lipid selected from DLin-DMA, DLin-K-DMA, DLin-KC2-DMA, DLin-MC3-DMA, 98N12-5, and C12-200); a structural lipid (e.g., distearoylphosphatidylcholine (DSPC)); cholesterol, and a polyethyleneglycol (PEG)-lipid (e.g., a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); dioleoylphosphatidylethanolamine (DOPE). In embodiments, the lipid nanoparticles comprise (a) a cationic lipid comprising from 50 mol % to 85 mol % of the total lipid present in the particle; (b) a non-cationic lipid comprising from 13 mol % to 49.5 mol % of the total lipid present in the particle; and (c) a conjugated lipid that inhibits aggregation of particles comprising from 0.5 mol % to 2 mol % of the total lipid present in the particle. In embodiments, the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

In any of the embodiments disclosed herein, the pharmaceutical composition is formulated for parenteral administration. In any of the embodiments disclosed herein, the pharmaceutical composition is formulated for topical administration

In one aspect, the present disclosure provides a pharmaceutical composition comprising the mmRNA of any embodiment disclosed herein, or an LNP comprising an mmRNA of any embodiment disclosed herein. In embodiments, the pharmaceutical composition is formulated for parenteral administration. In embodiments, the pharmaceutical composition intratumoral injection.

In embodiments, the pharmaceutical composition comprises a modified mRNA (mmRNA) encoding a heterologous chimeric protein having an amino acid sequence that has at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence selected from SEQ ID NOs: 80-93. In embodiments, the pharmaceutical composition is formulated as an LNP comprising an ionizable amino lipid, a phospholipid, a structural lipid and a PEG lipid.

In embodiments, the lipid nanoparticles comprise (a) a cationic lipid comprising from 50 mol % to 85 mol % of the total lipid present in the particle; (b) a non-cationic lipid comprising from 13 mol % to 49.5 mol % of the total lipid present in the particle; and (c) a conjugated lipid that inhibits aggregation of particles comprising from 0.5 mol % to 2 mol % of the total lipid present in the particle. In embodiments, the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

In embodiments, the pharmaceutical composition is formulated for parenteral administration. In embodiments, the pharmaceutical composition is formulated for topical, dermal, intradermal, intramuscular, intraperitoneal, intraarticular, intravenous, subcutaneous, intraarterial or transdermal administration. In embodiments, the pharmaceutical composition is formulated for topical administration.

In one aspect, the present disclosure provides a pharmaceutical composition comprising a pharmaceutically acceptable excipient or carrier, and the chimeric protein of any one of the embodiments disclosed herein, the isolated polynucleotide of any one of the embodiments disclosed herein, the vector of the embodiments disclosed herein, or the host cell of any of the embodiments disclosed herein. In embodiments, the pharmaceutical composition comprises the nucleic acid, e.g., the mmRNA of any one of the embodiments disclosed herein.

In embodiments, the carrier is a lipidoid, a liposome, a lipoplex, a lipid nanoparticle, a polymeric nanoparticle, a peptide, a protein, a cell, a nanoparticle mimic, a nanotube, or a conjugate. In embodiments, the pharmaceutical composition is formulated as a lipid nanoparticles (LNPs), a lipoplex, or a liposome. In embodiments, the pharmaceutical composition is formulated as a lipid nanoparticles (LNPs).

In embodiments, the lipid nanoparticles comprise lipids selected from an ionizable lipid (e.g., an ionizable cationic lipid selected from DLin-DMA, DLin-K-DMA, DLin-KC2-DMA, DLin-MC3-DMA, 98N12-5, and C12-200); a structural lipid (e.g., distearoylphosphatidylcholine (DSPC)); cholesterol, and a polyethyleneglycol (PEG)-lipid (e.g., a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); dioleoylphosphatidylethanolamine (DOPE); and the nucleic acid, e.g., the mmRNA.

In embodiments, the LNP comprises a molar ratio of about 20-60% ionizable amino lipid, about 5-25% phospholipid, about 25-55% structural lipid, and about 0.5-1.5% PEG lipid. In embodiments, the ionizable amino lipid comprises the following formula:

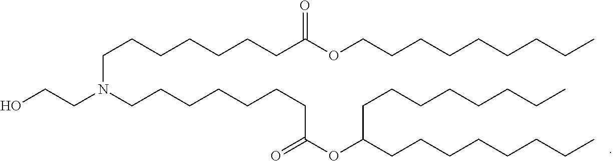 [SMILES: CCCCCCCCCOC(=O)CCCCCCCN(CCO)CCCCCCCC(=O)OC(CCCCCCCC)CCCCCCCC]

In embodiments, the lipid nanoparticles comprise lipids selected from an ionizable lipid; a structural lipid; cholesterol, and a polyethyleneglycol (PEG)-lipid; 1,2-dioleoyl-3-trimethylammoniumpropane (DOTAP); dioleoylphosphatidylethanolamine (DOPE); and the nucleic acid, e.g., the mmRNA. In embodiments, the ionizable lipid is an ionizable cationic lipid selected from DLin-DMA, DLin-K-DMA, DLin-KC2-DMA, DLin-MC3-DMA, 98N12-5, and C12-200. In embodiments, the polyethyleneglycol (PEG)-lipid is selected from a PEG-diacylglycerol (DAG), a PEG-dialkyloxypropyl (DAA), a PEG-phospholipid, a PEG-ceramide (Cer), or a mixture thereof, or a PEG-dilauryloxypropyl (e.g., C12, a PEG-dimyristyloxypropyl (C14), a PEG-dipalmityloxypropyl (C16), or a PEG-distearyloxypropyl (C18)).

In embodiments, the lipid nanoparticles comprise (a) a cationic lipid comprising from 50 mol % to 85 mol % of the total lipid present in the particle; (b) a non-cationic lipid comprising from 13 mol % to 49.5 mol % of the total lipid present in the particle; and (c) a conjugated lipid that inhibits aggregation of particles comprising from 0.5 mol % to 2 mol % of the total lipid present in the particle. In embodiments, the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

In embodiments, the isolated polynucleotide is or comprises a conjugated polynucleotide sequence that is introduced into cells by various transfection methods such as, e.g., methods that employ lipid particles. In embodiments, a composition, including a gene transfer construct, comprises a delivery particle. In embodiments, the delivery particle comprises a lipid-based particle (e.g., a lipid nanoparticle (LNP)), cationic lipid, or a biodegradable polymer). Lipid nanoparticle (LNP) delivery of gene transfer construct provides certain advantages, including transient, non-integrating expression to limit potential off-target events and immune responses, and efficient delivery with the capacity to transport large cargos. LNPs have been used for delivery of small interfering RNA (siRNA) and mRNA, and for in vitro and in vivo delivering CRISPR/Cas9 components to hepatocytes and the liver. For example, U.S. Pat. No. 10,195,291 describes the use of LNPs for delivery of RNA interference (RNAi) therapeutic agents.

In embodiments, the composition in accordance with embodiments of the present disclosure is in the form of a LNP. In embodiments, the LNP comprises one or more lipids selected from 1,2-dioleoyl-3-trimethylammonium propane (DOTAP); N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy(polyethylene glycol)-2000](DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC).

In embodiments, the composition can have a lipid and a polymer in various ratios, wherein the lipid can be selected from, e.g., DOTAP, DC-Chol, PC, Triolein, DSPE-PEG, and wherein the polymer can be, e.g., PEI or Poly Lactic-co-Glycolic Acid (PLGA). Any other lipid and polymer can be used additionally or alternatively. In embodiments, the ratio of the lipid and the polymer is about 0.5:1, or about 1:1, or about 1:1.5, or about 1:2, or about 1:2.5, or about 1:3, or about 3:1, or about 2.5:1, or about 2:1, or about 1.5:1, or about 1:1, or about 1:0.5.

In embodiments, the LNP comprises a cationic lipid, non-limiting examples of which include N,N-dioleyl-N,N-dimethylammonium chloride (DODAC), N,N-distearyl-N,N-dimethylammonium bromide (DDAB), N-(I-(2,3-dioleoyloxy)propyl)-N,N,N-trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino)butanoate (MC3), 1,1′-(2-(4-(2-((2-(bis(2-′)amino)ethyl)(2 hydroxydodecyl)amino)ethyl) piperazin-1-yl)ethylazanediyl)didodecan-2-ol (Tech G1), 1,2-Dilinoleyloxo-3-(2-N,N-dimethylamino) ethoxypropane (DLin-EG-DMA), or a mixture thereof.

In embodiments, the LNP comprises one or more molecules selected from polyethylenimine (PEI) and poly(lactic-co-glycolic acid) (PLGA), and N-Acetylgalactosamine (GalNAc), which are suitable for hepatic delivery. In embodiments, the LNP comprises a hepatic-directed compound as described, e.g., in U.S. Pat. No. 5,985,826, which is incorporated by reference herein in its entirety. GalNAc is known to target Asialoglycoprotein Receptor (ASGPR) expressed on mammalian hepatic cells. See Hu et al. Protein Pept Lett. 2014; 21(10):1025-30.

In some examples, the isolated polynucleotide can be formulated or complexed with PEI or a derivative thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

In embodiments, the LNP is a conjugated lipid, non-limiting examples of which include a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (C12, a PEG-dimyristyloxypropyl (C14), a PEG-dipalmityloxypropyl (C16), or a PEG-distearyloxypropyl (C18).

In embodiments, the LNP formulations may further contain a phosphate conjugate, which can increase in vivo circulation times and/or increase the targeted delivery of the nanoparticle. Phosphate conjugates can be made by the methods described in, e.g., PCT International Publication No. WO2013033438 or U.S. Pub. No. US20130196948. The LNP formulation can also contain a polymer conjugate (e.g., a water soluble conjugate) as described in, e.g., U.S. Publication Nos. US20130059360, US20130196948, and US20130072709, each of the references is herein incorporated by reference in its entirety.

In embodiments, the LNP formulations may comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., PCT International Publication No. WO2012109121, herein incorporated by reference in its entirety). In embodiments, the LNP formulations can be coated with a surfactant or polymer to improve the delivery of the particle. In some embodiments, the LNP can be coated with a hydrophilic coating such as, but not limited to, PEG coatings and/or coatings that have a neutral surface charge as described in U.S. Publication No. US20130183244, herein incorporated by reference in its entirety. In embodiments, the LNP formulations can be engineered to alter the surface properties of particles so that the lipid nanoparticles can penetrate the mucosal barrier as described in U.S. Pat. No. 8,241,670 or PCT International Publication No. WO2013110028, each of which is herein incorporated by reference in its entirety. In embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., PCT International Publication No. WO2013110028, herein incorporated by reference in its entirety.

In embodiments, an mmRNA described herein is formulated as a solid lipid nanoparticle (SLN), which can be spherical with an average diameter between 10 to 1000 nm. SLN possess a solid lipid core matrix that can solubilize lipophilic molecules and can be stabilized with surfactants and/or emulsifiers. Exemplary SLN can be those as described in PCT International Publication No. WO2013105101, herein incorporated by reference in its entirety.

In embodiments, a nanoparticle is a particle having a diameter of less than about 1000 nm. In embodiments, nanoparticles of the present disclosure have a greatest dimension (e.g., diameter) of about 500 nm or less, or about 400 nm or less, or about 300 nm or less, or about 200 nm or less, or about 100 nm or less. In embodiments, nanoparticles of the present disclosure have a greatest dimension ranging between about 50 nm and about 150 nm, or between about 70 nm and about 130 nm, or between about 80 nm and about 120 nm, or between about 90 nm and about 110 nm. In embodiments, the nanoparticles of the present disclosure have a greatest dimension (e.g., a diameter) of about 100 nm.

In embodiments, the therapeutic nanoparticle mRNA can be formulated for sustained release, which, as used herein, refers to a pharmaceutical composition or compound that conforms to a release rate over a specific period of time. In embodiments, the period of time may include, but is not limited to, hours, days, weeks, months and years. As a non-limiting example, the sustained release nanoparticle of the mRNAs described herein can be formulated as disclosed in PCT International Publication No. WO2010075072 and U.S. Publication Nos. US20100216804, US20110217377, US20120201859 and US20130150295, each of which is herein incorporated by reference in their entirety.

In embodiments, the isolated polynucleotide or mmRNA (and/or additional agents) are included various formulations. Any isolated polynucleotide or mmRNA (and/or additional agents) described herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In embodiments, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

In embodiments, the present disclosure provides an expression vector, comprising a nucleic acid encoding the chimeric protein described herein. In embodiments, the expression vector comprises DNA or RNA. In embodiments, the expression vector is a mammalian expression vector.

Both prokaryotic and eukaryotic vectors can be used for expression of the chimeric protein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, lpp, phoA, recA, tac, T3, T7 and APL. Non-limiting examples of prokaryotic expression vectors may include the λgt vector series such as λgt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful. A variety of regulatory regions can be used for expression of the chimeric proteins in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the chimeric proteins in recombinant host cells.

In embodiments, expression vectors of the disclosure comprise a nucleic acid encoding the chimeric proteins (and/or additional agents), or a complement thereof, operably linked to an expression control region, or complement thereof, that is functional in a mammalian cell. The expression control region is capable of driving expression of the operably linked blocking and/or stimulating agent encoding nucleic acid such that the blocking and/or stimulating agent is produced in a human cell transformed with the expression vector.

Expression control regions are regulatory polynucleotides (sometimes referred to herein as elements), such as promoters and enhancers, that influence expression of an operably linked nucleic acid. An expression control region of an expression vector of the disclosure is capable of expressing operably linked encoding nucleic acid in a human cell. In embodiments, the cell is an epithelial cell. In embodiments, the cell is located in or near a lesion disorder caused by or associated with inflammation of the integumentary system. In embodiments, the cell is a non-tumor cell. In embodiments, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

Expression control regions and locus control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

As used herein, “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter. Examples of promoters that are expressed in the integumentary system include a keratin 5 (K5) promoter, a keratin 6 (K6) promoter, a keratin 14 (K14) promoter, a keratin 16 (K16) promoter, an alpha-1(I) collagen promoter, a filaggrin promoter, a loricrin promoter, an involucrin promoter, a tyrosinase promoter, and an αV integrin promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs.

There are a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a cell surface membrane protein from cells located in or near a lesion disorder caused by or associated with inflammation of the integumentary system. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), FIp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences encoding the chimeric proteins including CRISPR/CAS9, zinc finger, TALEN, and meganuclease gene-editing technologies.

In one aspect, the disclosure provides expression vectors for the expression of the chimeric proteins (and/or additional agents) that are viral vectors. Many viral vectors useful for gene therapy are known (see, e.g., Lundstrom, Trends Biotechnol., 21: 1 17, 122, 2003. Illustrative viral vectors include those selected from Antiviruses (LV), retroviruses (RV), adenoviruses (AV), adeno-associated viruses (AAV), and a viruses, though other viral vectors may also be used. For in vivo uses, viral vectors that do not integrate into the host genome are suitable for use, such as a viruses and adenoviruses. Illustrative types of a viruses include Sindbis virus, Venezuelan equine encephalitis (VEE) virus, and Semliki Forest virus (SFV). For in vitro uses, viral vectors that integrate into the host genome are suitable, such as retroviruses, AAV, and Antiviruses. In embodiments, the disclosure provides methods of transducing a human cell in vivo, comprising contacting a cell that is located in or near a lesion disorder caused by or associated with inflammation of the integumentary system in vivo with a viral vector of the disclosure.

In embodiments, the present disclosure provides a host cell, comprising the expression vector comprising the chimeric protein described herein. In embodiments, the present disclosure provides a host cell comprising an RNA (without limitations, e.g., mmRNA) encoring the chimeric protein of any of the embodiments disclosed herein.

Expression vectors can be introduced into host cells for producing the present chimeric proteins. Cells may be cultured in vitro or genetically engineered, for example. Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al., J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); DG44 CHO cells, CHO-K1 cells, mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the chimeric proteins described herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, and human small cell lung carcinoma cell lines, SCLC #2 and SCLC #7.

Host cells can be obtained from normal or affected subjects, including healthy humans, patients suffering from inflammation of the integumentary system, and patients with an infectious disease, private laboratory deposits, public culture collections such as the American Type Culture Collection, or from commercial suppliers.

Cells that can be used for production of the present chimeric proteins in vitro, ex vivo, and/or in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells (e.g., as obtained from bone marrow), umbilical cord blood, peripheral blood, fetal liver, etc. The choice of cell type depends on the type of the disease or disorder caused by or associated with inflammation of the integumentary system being treated or prevented, and can be determined by one of skill in the art.

Where necessary, the formulations comprising the isolated polynucleotide or mmRNA (and/or additional agents) can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The formulations comprising the isolated polynucleotide or mmRNA (and/or additional agents) of the present disclosure may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art)

In embodiments, any isolated polynucleotide or mmRNA (and/or additional agents) described herein is formulated in accordance with routine procedures as a composition adapted for a mode of administration described herein.

Any isolated polynucleotide or mmRNA (and/or additional agents) described herein can be administered orally. Such chimeric proteins (and/or additional agents) can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

The dosage of any isolated polynucleotide or mmRNA (and/or additional agents) described herein as well as the dosing schedule can depend on various parameters, including, but not limited to, the disease being treated, the subject's general health, and the administering physician's discretion. Any chimeric protein described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional agent, to a subject in need thereof. In embodiments any chimeric protein and additional agent described herein are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days part, 4 days apart, 5 days apart, 6 days apart, 1 week apart, 2 weeks apart, 3 weeks apart, or 4 weeks apart.

The dosage of any isolated polynucleotide or mmRNA (and/or additional agents) described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In embodiments, delivery can be in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989).

Any isolated polynucleotide or mmRNA (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In embodiments, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In embodiments, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of any isolated polynucleotide or mmRNA (and/or additional agents) described herein can, independently, be one to four times daily or one to four times per month or one to six times per year or once every two, three, four or five years. Administration can be for the duration of one day or one month, two months, three months, six months, one year, two years, three years, and may even be for the life of the subject.

The dosage regimen utilizing any isolated polynucleotide or mmRNA (and/or additional agents) described herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the disclosure employed. Any isolated polynucleotide or mmRNA (and/or additional agents) described herein can be administered in a single daily dose, or the total daily dosage can be administered in divided doses of two, three or four times daily. Furthermore, any isolated polynucleotide or mmRNA (and/or additional agents) described herein can be administered continuously rather than intermittently throughout the dosage regimen.

In one aspect, the present disclosure provides a host cell comprising the polynucleotide of any of the embodiments disclosed herein.

In one aspect, the present disclosure provides a host cell comprising the vector of the embodiments disclosed herein. In one aspect, the present disclosure provides a host cell comprising an RNA (without limitations, e.g., mmRNA) encoring the chimeric protein of any of the embodiments disclosed herein

Diseases/Disorders that May be Treated with the Nucleic Acids Encoding the Chimeric Proteins of the Present Disclosure

The methods disclosed herein are useful for delivery of chimeric proteins for treating diseases that include but are not limited to autoimmunity, fibrotic disease, and other inflammatory disorders.

The isolated polynucleotide or mmRNA encoding the chimeric proteins disclosed herein finds use in methods for treating both advanced solid tumors and advanced lymphomas. These tumor types include: melanoma, non-small cell lung cancer (squamous, adeno, adeno-squamous), urothelial cancer, renal cell cancer, squamous cell cervical cancer, gastric or gastro-esophageal junction adenocarcinoma, squamous cell carcinoma of the anus, squamous cell carcinoma of the head and neck, squamous cell carcinoma of the skin, and microsatellite instability high or mismatch repair deficient solid tumors excluding central nervous system (CNS) tumors. Other tumors of interest include Hodgkin's lymphoma (HL), diffuse large B cell lymphoma, acute myeloid leukemia (AML) and high-risk myelodysplastic syndromes (HR-MDS).

In embodiments, the cancer comprises an advanced lymphoma. In embodiments, the cancer comprises acute myeloid leukemia (AML). In embodiments, the cancer comprises p53 mutant AML. In embodiments, the cancer comprises a high-risk myelodysplastic syndrome (HR-MDS).

Aspects of the present disclosure provide methods of treating cancer. The methods comprise a step of administering to a subject in need thereof an effective amount of a chimeric protein, e.g., in a pharmaceutical composition, as disclosed herein.

It is often desirable to enhance immune stimulatory signal transmission to boost an immune response, for instance to enhance a patient's anti-tumor immune response.

In embodiments, the present isolated polynucleotide or mmRNA encoding the chimeric proteins are capable of, or can be used in methods comprising, modulating the amplitude of an immune response, e.g., modulating the level of effector output. In embodiments, e.g., when used for the treatment of cancer, the present isolated polynucleotide or mmRNA encoding the chimeric proteins alter the extent of immune stimulation as compared to immune inhibition to increase the amplitude of a T cell response, including, without limitation, stimulating increased levels of cytokine production, proliferation or target killing potential. In embodiments, the patient's T cells are activated and/or stimulated by the chimeric protein, with the activated T cells being capable of dividing and/or secreting cytokines.

Cancers or tumors refer to an uncontrolled growth of cells and/or abnormal increased cell survival and/or inhibition of apoptosis which interferes with the normal functioning of the bodily organs and systems. Included are benign and malignant cancers, polyps, hyperplasia, as well as dormant tumors or micrometastases. Also, included are cells having abnormal proliferation that is not impeded by the immune system (e.g., virus infected cells). The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in a local area, forming a new tumor, which may be a local metastasis. The cancer may also be caused by a cancer cell that acquires the ability to penetrate the walls of lymphatic and/or blood vessels, after which the cancer cell is able to circulate through the bloodstream (thereby being a circulating tumor cell) to other sites and tissues in the body. The cancer may be due to a process such as lymphatic or hematogenous spread. The cancer may also be caused by a tumor cell that comes to rest at another site, re-penetrates through the vessel or walls, continues to multiply, and eventually forms another clinically detectable tumor. The cancer may be this new tumor, which may be a metastatic (or secondary) tumor.

The cancer may be caused by tumor cells that have metastasized, which may be a secondary or metastatic tumor. The cells of the tumor may be like those in the original tumor. As an example, if a breast cancer or colon cancer metastasizes to the liver, the secondary tumor, while present in the liver, is made up of abnormal breast or colon cells, not of abnormal liver cells. The tumor in the liver may thus be a metastatic breast cancer or a metastatic colon cancer, not liver cancer.

The cancer may have an origin from any tissue. The cancer may originate from melanoma, colon, breast, or prostate, and thus may be made up of cells that were originally skin, colon, breast, or prostate, respectively. The cancer may also be a hematological malignancy, which may be leukemia or lymphoma. The cancer may invade a tissue such as liver, lung, bladder, or intestinal.

In embodiments, the chimeric protein is used to treat a subject that has a treatment-refractory cancer. In embodiments, the chimeric protein is used to treat a subject that is refractory to one or more immune-modulating agents. For example, in embodiments, the chimeric protein is used to treat a subject that presents no response to treatment, or whose disease progresses, after 12 weeks or so of treatment. For instance, in embodiments, the subject is refractory to one or more CD172a (SIRPα) and/or CD47 agents, including, for example, Magrolimab (5F9), Hu5F9-G4, CC-90002, Ti-061, SRF231, TTI-621, TTI-622, or ALX148 refractory patients. For instance, in embodiments, the subject is refractory to an anti-CTLA-4 agent, e.g., ipilimumab (YERVOY)-refractory patients (e.g., melanoma patients). Accordingly, in embodiments the present disclosure provides methods of cancer treatment that rescue patients that are non-responsive to various therapies, including monotherapy of one or more immune-modulating agents.

In embodiments, the present disclosure provides isolated polynucleotide or mmRNA encoding the chimeric proteins, which target a cell or tissue within the tumor microenvironment. In embodiments, the cell or tissue within the tumor microenvironment expresses one or more targets or binding partners of the chimeric protein. The tumor microenvironment refers to the cellular milieu, including cells, secreted proteins, physiological small molecules, and blood vessels in which the tumor exists. In embodiments, the cells or tissue within the tumor microenvironment are one or more of: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor. In embodiments, the present chimeric protein targets a cancer cell. In embodiments, the cancer cell expresses one or more of targets or binding partners of the chimeric protein.

The activation of regulatory T cells is critically influenced by costimulatory and co-inhibitory signals. Two major families of costimulatory molecules include the B7 and the tumor necrosis factor (TNF) families. These molecules bind to receptors on T cells belonging to the CD28 or TNF receptor families, respectively. Many well-defined co-inhibitors and their receptors belong to the B7 and CD28 families.

In embodiments, an immune stimulatory signal refers to a signal that enhances an immune response. For example, in the context of oncology, such signals may enhance antitumor immunity. For instance, without limitation, immune stimulatory signal may be identified by directly stimulating proliferation, cytokine production, killing activity, or phagocytic activity of leukocytes. For example, a chimeric protein may directly stimulate the proliferation and cytokine production of individual T cell subsets. Another example includes direct stimulation of an immune inhibitory cell with through a receptor that inhibits the activity of such an immune suppressor cell. This would include, for example, stimulation of CD4+FoxP3+ regulatory T cells, which would reduce the ability of those regulatory T cells to suppress the proliferation of conventional CD4+ or CD8+ T cells. In another example, this would include stimulation of CD40 on the surface of an antigen presenting cell, causing activation of antigen presenting cells including enhanced ability of those cells to present antigen in the context of appropriate native costimulatory molecules, including those in the B7 or TNF superfamily. In another example, the chimeric protein causes activation of the lymphoid cell and/or production of pro-inflammatory cytokines or chemokines to further stimulate an immune response, optionally within a tumor.

In embodiments, the present isolated polynucleotide or mmRNA encoding the chimeric proteins are capable of, or find use in methods involving, enhancing, restoring, promoting and/or stimulating immune modulation. In embodiments, the present isolated polynucleotide or mmRNA encoding the chimeric proteins described herein, restore, promote and/or stimulate the activity or activation of one or more immune cells against tumor cells including, but not limited to: T cells, cytotoxic T lymphocytes, T helper cells, natural killer (NK) cells, natural killer T (NKT) cells, anti-tumor macrophages (e.g., M1 macrophages), B cells, and dendritic cells. In embodiments, the present isolated polynucleotide or mmRNA encoding the chimeric proteins enhance, restore, promote and/or stimulate the activity and/or activation of T cells, including, by way of a non-limiting example, activating and/or stimulating one or more T-cell intrinsic signals, including a pro-survival signal; an autocrine or paracrine growth signal; a p38 MAPK-, ERK-, STAT-, JAK-, AKT- or PI3K-mediated signal; an anti-apoptotic signal; and/or a signal promoting and/or necessary for one or more of: pro-inflammatory cytokine production or T cell migration or T cell tumor infiltration.

In embodiments, the present isolated polynucleotide or mmRNA encoding the chimeric proteins are capable of, or find use in methods involving, causing an increase of one or more of T cells (including without limitation cytotoxic T lymphocytes, T helper cells, natural killer T (NKT) cells), B cells, natural killer (NK) cells, natural killer T (NKT) cells, dendritic cells, monocytes, and macrophages (e.g., one or more of M1 and M2) into a tumor or the tumor microenvironment. In embodiments, the chimeric protein enhances recognition of tumor antigens by CD8+ T cells, particularly those T cells that have infiltrated into the tumor microenvironment. In embodiments, the present chimeric protein induces CD19 expression and/or increases the number of CD19 positive cells (e.g., CD19 positive B cells). In embodiments, the present chimeric protein induces IL-15Rα expression and/or increases the number of IL-15Rα positive cells (e.g., IL-15Rα positive dendritic cells).

In embodiments, the present isolated polynucleotide or mmRNA encoding the chimeric proteins are capable of, or find use in methods involving, inhibiting and/or causing a decrease in immunosuppressive cells (e.g., myeloid-derived suppressor cells (MDSCs), regulatory T cells (Tregs), tumor associated neutrophils (TANs), M2 macrophages, and tumor associated macrophages (TAMs)), and particularly within the tumor and/or tumor microenvironment (TME). In embodiments, the present therapies may alter the ratio of M1 versus M2 macrophages in the tumor site and/or TME to favor M1 macrophages.

In embodiments, the present isolated polynucleotide or mmRNA encoding the chimeric proteins are able to increase the serum levels of various cytokines including, but not limited to, one or more of IFNγ, TNFα, IL-2, IL-4, IL-5, IL-9, IL-10, IL-13, IL-17A, IL-17F, and IL-22. In embodiments, the present isolated polynucleotide or mmRNA encoding the chimeric proteins are capable of enhancing IL-2, IL-4, IL-5, IL-10, IL-13, IL-17A, IL-22, or IFNγ in the serum of a treated subject. In embodiments, the present isolated polynucleotide or mmRNA encoding the chimeric proteins do not increase the serum levels of certain cytokines. In embodiments, the present isolated polynucleotide or mmRNA encoding the chimeric proteins do not increase the serum levels of IL-6 and/or TNFα. In embodiments, the present isolated polynucleotide or mmRNA encoding the chimeric proteins do not increase the serum levels of f IL-6 and/or TNFα in the serum of a treated subject. In embodiments, the present isolated polynucleotide or mmRNA encoding the chimeric proteins do not increase the serum levels of f IL-6 and/or TNFα in the serum of a treated subject, while increasing the levels of other cytokines, including but not limited to, CCL2, IL-8 and CXCL9 in serum of a treated subject. Detection of such a cytokine response may provide a method to determine the optimal dosing regimen for the indicated chimeric protein.

In a chimeric protein of the present disclosure, the chimeric protein is capable of increasing or preventing a decrease in a sub-population of CD4+ and/or CD8+ T cells.

In a chimeric protein of the present disclosure, the chimeric protein is capable of enhancing tumor killing activity by T cells.

In embodiments, the chimeric protein activates the human subject's T cells when bound by the CD40L domain of the chimeric protein and (a) one or more tumor cells are prevented from transmitting an immunosuppressive signal when bound by the first domain of the chimeric protein, (b) a quantifiable cytokine response in the peripheral blood of the subject is achieved, and/or (c) tumor growth is reduced in the subject in need thereof as compared to a subject treated with CD40 agonist antibodies and/or CD47 blocking antibodies.

In embodiments, the present isolated polynucleotide or mmRNA encoding the chimeric proteins inhibit, block and/or reduce cell death of an anti-tumor CD8+ and/or CD4+ T cell; or stimulate, induce, and/or increase cell death of a pro-tumor T cell. T cell exhaustion is a state of T cell dysfunction characterized by progressive loss of proliferative and effector functions, culminating in clonal deletion. Accordingly, a pro-tumor T cell refers to a state of T cell dysfunction that arises during many chronic infections, inflammatory diseases, and cancer. This dysfunction is defined by poor proliferative and/or effector functions, sustained expression of inhibitory receptors and a transcriptional state distinct from that of functional effector or memory T cells. Exhaustion prevents optimal control of infection and tumors. Illustrative pro-tumor T cells include, but are not limited to, Tregs, CD4+ and/or CD8+ T cells expressing one or more checkpoint inhibitory receptors, Th2 cells and Th17 cells. Checkpoint inhibitory receptors refer to receptors expressed on immune cells that prevent or inhibit uncontrolled immune responses. In contrast, an anti-tumor CD8+ and/or CD4+ T cell refers to T cells that can mount an immune response to a tumor.

In embodiments, the present isolated polynucleotide or mmRNA encoding the chimeric proteins are capable of, and can be used in methods comprising, increasing a ratio of effector T cells to regulatory T cells. Illustrative effector T cells include ICOS⁺ effector T cells; cytotoxic T cells (e.g., αβ TCR, CD3⁺, CD8⁺, CD45RO⁺); CD4⁺ effector T cells (e.g., αβ TCR, CD3⁺, CD4⁺, CCR7⁺, CD62Lhi, IL⁻7R/CD127⁺); CD8⁺ effector T cells (e.g., αβ TCR, CD3⁺, CD8⁺, CCR7⁺, CD62Lhi, IL-7R/CD127⁺); effector memory T cells (e.g., CD62Llow, CD44⁺, TCR, CD3⁺, IL⁻7R/CD127⁺, IL-15R⁺, CCR7low); central memory T cells (e.g., CCR7⁺, CD62L⁺, CD27⁺; or CCR7hi, CD44⁺, CD62Lhi, TCR, CD3⁺, IL-7R/CD127⁺, IL-15R⁺); CD62L⁺ effector T cells; CD8⁺ effector memory T cells (TEM) including early effector memory T cells (CD27⁺CD62L⁻) and late effector memory T cells (CD27⁻ CD62L⁻) (TemE and TemL, respectively); CD127(⁺)CD25(low/−) effector T cells; CD127(⁻)CD25(⁻) effector T cells; CD8⁺ stem cell memory effector cells (TSCM) (e.g., CD44(low)CD62L(high)CD122(high)sca(⁺)); TH1 effector T-cells (e.g., CXCR3⁺, CXCR6⁺ and CCR5⁺; or αβ TCR, CD3⁺, CD4⁺, IL-12R⁺, IFNγR⁺, CXCR3⁺), TH2 effector T cells (e.g., CCR3⁺, CCR4⁺ and CCR8⁺; or αβ TCR, CD3⁺, CD4⁺, IL-4R⁺, IL-33R⁺, CCR4⁺, IL-17RB⁺, CRTH2⁺); TH9 effector T cells (e.g., αβ TCR, CD3⁺, CD4⁺); TH17 effector T cells (e.g., αβ TCR, CD3⁺, CD4⁺, IL-23R⁺, CCR6⁺, IL-1R⁺); CD4⁺CD45RO⁺CCR7⁺ effector T cells, CD4⁺CD45RO⁺CCR7(⁻) effector T cells; and effector T cells secreting IL-2, IL-4 and/or IFN-γ. Illustrative regulatory T cells include ICOS⁺ regulatory T cells, CD4⁺CD25⁺FOXP3⁺ regulatory T cells, CD4⁺CD25⁺ regulatory T cells, CD4⁺CD25⁻ regulatory T cells, CD4⁺CD25high regulatory T cells, TIM-3⁺CD172a (SIRPα)⁺ regulatory T cells, lymphocyte activation gene-3 (LAG-3)⁺ regulatory T cells, CTLA-4/CD152⁺ regulatory T cells, neuropilin-1 (Nrp-1)⁺ regulatory T cells, CCR4⁺CCR8⁺ regulatory T cells, CD62L (L-selectin)⁺ regulatory T cells, CD45RBlow regulatory T cells, CD127low regulatory T cells, LRRC32/GARP⁺ regulatory T cells, CD39⁺ regulatory T cells, GITR⁺ regulatory T cells, LAP⁺ regulatory T cells, 1B11⁺ regulatory T cells, BTLA⁺ regulatory T cells, type 1 regulatory T cells (Tr1 cells), T helper type 3 (Th3) cells, regulatory cell of natural killer T cell phenotype (NKTregs), CD8⁺ regulatory T cells, CD8⁺CD28⁻ regulatory T cells and/or regulatory T-cells secreting IL-10, IL-35, TGF-3, TNF-α, Galectin-1, IFN-γ and/or MCP1.

In embodiments, the chimeric protein of the invention causes an increase in effector T cells (e.g., CD4+CD25− T cells).

In embodiments, the chimeric protein causes a decrease in regulatory T cells (e.g., CD4+CD25+ T cells).

In embodiments, the chimeric protein generates a memory response which may, e.g., be capable of preventing relapse or protecting the animal from a recurrence and/or preventing, or reducing the likelihood of, metastasis. Thus, an animal treated with the chimeric protein is later able to attack tumor cells and/or prevent development of tumors when rechallenged after an initial treatment with the chimeric protein. Accordingly, a chimeric protein of the present disclosure stimulates both active tumor destruction and also immune recognition of tumor antigens, which are essential in programming a memory response capable of preventing relapse.

In embodiments, the chimeric protein is capable of causing activation of antigen presenting cells. In embodiments, the chimeric protein is capable enhancing the ability of antigen presenting cells to present antigen.

In embodiments, the present isolated polynucleotide or mmRNA encoding the chimeric proteins are capable of, and can be used in methods comprising, transiently stimulating effector T cells for longer than about 12 hours, about 24 hours, about 48 hours, about 72 hours or about 96 hours or about 1 week or about 2 weeks. In embodiments, the transient stimulation of effector T cells occurs substantially in a patient's bloodstream or in a particular tissue/location including lymphoid tissues such as for example, the bone marrow, lymph-node, spleen, thymus, mucosa-associated lymphoid tissue (MALT), non-lymphoid tissues, or in the tumor microenvironment.

In a chimeric protein of the present disclosure, the present chimeric protein unexpectedly provides binding of the extracellular domain components to their respective binding partners with slow off rates (Kd or K_{off}). In embodiments, this provides an unexpectedly long interaction of the receptor to ligand and vice versa. Such an effect allows for a longer positive signal effect, e.g., increase in or activation of immune stimulatory signals. For example, the present chimeric protein, e.g., via the long off rate binding allows sufficient signal transmission to provide immune cell proliferation, allow for anti-tumor attack, allows sufficient signal transmission to provide release of stimulatory signals, e.g., cytokines.

In a chimeric protein of the present disclosure, the chimeric protein is capable of forming a stable synapse between cells. The stable synapse of cells promoted by the chimeric proteins (e.g., between cells bearing negative signals) provides spatial orientation to favor tumor reduction—such as positioning the T cells to attack tumor cells and/or sterically preventing the tumor cell from delivering negative signals, including negative signals beyond those masked by the chimeric protein of the invention. In embodiments, this provides longer on-target (e.g., intratumoral) half-life (t_{1/2}) as compared to serum t_{1/2} of the chimeric proteins. Such properties could have the combined advantage of reducing off-target toxicities associated with systemic distribution of the chimeric proteins.

In embodiments, the isolated polynucleotide or mmRNA encoding the chimeric protein is capable of providing a sustained immunomodulatory effect.

The present isolated polynucleotide or mmRNA encoding the chimeric proteins provide synergistic therapeutic effects (e.g., anti-tumor effects) as it allows for improved site-specific interplay of two immunotherapy agents. In embodiments, the present isolated polynucleotide or mmRNA encoding the chimeric proteins provide the potential for reducing off-site and/or systemic toxicity.

In embodiments, the present chimeric protein exhibit enhanced safety profiles. In embodiments, the present chimeric protein exhibit reduced toxicity profiles. For example, administration of the present isolated polynucleotide or mmRNA encoding the chimeric proteins may result in reduced side effects such as one or more of diarrhea, inflammation (e.g., of the gut), or weight loss, which occur following administration of antibodies directed to the ligand(s)/receptor(s) targeted by the extracellular domains of the present chimeric proteins. In embodiments, the present chimeric protein provides improved safety, as compared to antibodies directed to the ligand(s)/receptor(s) targeted by the extracellular domains of the present chimeric proteins, yet, without sacrificing efficacy.

In embodiments, the present isolated polynucleotide or mmRNA encoding the chimeric proteins provide reduced side-effects, e.g., GI complications, relative to current immunotherapies, e.g., antibodies directed to ligand(s)/receptor(s) targeted by the extracellular domains of the present chimeric proteins. Illustrative GI complications include abdominal pain, appetite loss, autoimmune effects, constipation, cramping, dehydration, diarrhea, eating problems, fatigue, flatulence, fluid in the abdomen or ascites, gastrointestinal (GI) dysbiosis, GI mucositis, inflammatory bowel disease, irritable bowel syndrome (IBS-D and IBS-C), nausea, pain, stool or urine changes, ulcerative colitis, vomiting, weight gain from retaining fluid, and/or weakness.

### EXAMPLES

The examples herein are provided to illustrate advantages and benefits of the present disclosure and to further assist a person of ordinary skill in the art with preparing or using the isolated polynucleotide or mmRNA encoding the chimeric proteins disclosed herein of the present disclosure. The examples herein are also presented in order to more fully illustrate the preferred aspects of the present disclosure. The examples should in no way be construed as limiting the scope of the present disclosure, as defined by the appended claims. The examples can include or incorporate any of the variations, aspects or embodiments of the present disclosure described above. The variations, aspects or embodiments described above may also further each include or incorporate the variations of any or all other variations, aspects or embodiments of the present disclosure.

### Example 1: In Vitro Expression of the TIGIT-Fc-GITRL Chimeric Protein Upon Transfection with a Modified mRNA

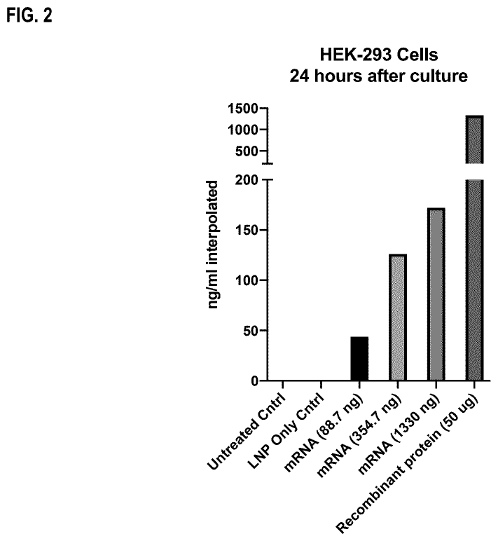

HEK293T cells were transfected with 88.7, 354.9, or 1330 μg a lipid nanoparticle (LNP) comprising a modified mRNA (mmRNA) encoding the murine TIGIT-Fc-GITRL chimeric protein. Untreated HEK293T cells and HEK293T cells transfected with LNP only were used as negative controls. Cell culture supernatants recovered after 24 hours for analysis. To analyze the expression of the TIGIT-Fc-GITRL chimeric protein, a dual antibody Meso Scale Discovery (MSD) ELISA assay was carried out. Briefly, the MSD GOLD 96-well Streptavidin SECTOR plates were coated with biotinylated anti-mouse GITRL goat polyclonal antibody (R&D Systems #BAF2177) and increasing amounts of the cell culture supernatants were added to the plate for capture by the plate-bound anti-mouse GITRL goat polyclonal antibody. A sample spiked with 50 μg of recombinant mouse TIGIT-Fc-GITRL protein was included as a positive control. Binding of the TIGIT-Fc-GITRL chimeric protein to the anti-mouse GITRL goat polyclonal antibody was detected using anti-mouse TIGIT rabbit IgG recombinant mAb (R&D Systems #MAB72671). A secondary SULFO TAG anti-Rabbit antibody was used to detect binding of anti-TIGIT to the mTIGIT-Fc-GITRL fusion protein. This assay detects the intact TIGIT-Fc-GITRL chimeric protein because detection requires simultaneous binding to anti-GITRL and anti-TIGIT antibodies. As shown in FIG. 2, a dose-dependent increase in the expression of the TIGIT-Fc-GITRL chimeric protein was observed.

These results demonstrate, inter alia, that the modified mRNA encoding the mouse TIGIT-Fc-GITRL chimeric protein could drive the expression of the TIGIT-Fc-GITRL chimeric protein in vitro.

### Example 2: In Vivo Nucleic Acid-Based Delivery of the SIRPα-Fc-CD40L Chimeric Protein

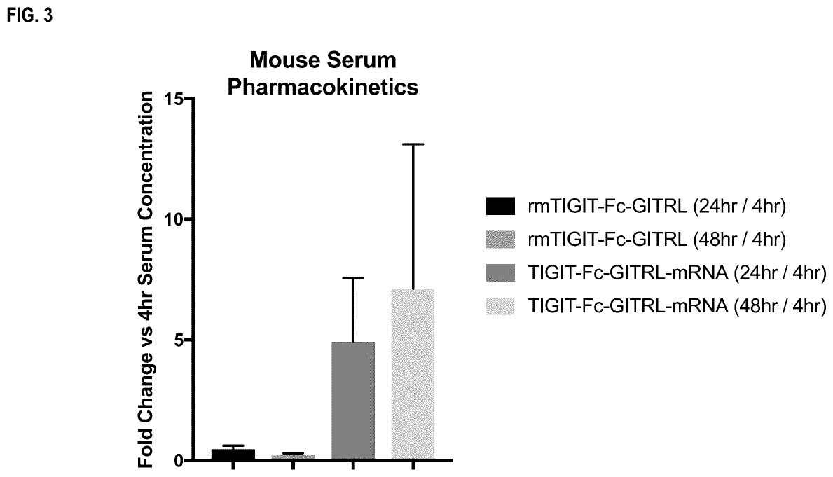

Mice were injected IV with 3.5 μg of mTIGIT-Fc-LIGHT mRNA formulated with in vivo-jetRNA lipid-based delivery reagent (Polyplus) or 50 μg of purified recombinant mTIGIT-Fc-GITRL chimeric protein. The expression of the TIGIT-Fc-GITRL chimeric protein was performed using a dual antibody Meso Scale Discovery (MSD) ELISA assay. Briefly, the MSD GOLD 96-well Streptavidin SECTOR plates were coated with biotinylated anti-mouse GITRL goat polyclonal antibody (R&D Systems #BAF2177) and increasing amounts of the cell culture supernatants were added to the plate for capture by the plate-bound anti-mouse GITRL goat polyclonal antibody. A sample spiked with 50 μg of recombinant mouse TIGIT-Fc-GITRL protein was included as a positive control. Binding of the TIGIT-Fc-GITRL chimeric protein to the anti-mouse GITRL goat polyclonal antibody was detected using anti-mouse TIGIT rabbit IgG recombinant mAb (R&D Systems #MAB72671). A secondary SULFO TAG anti-Rabbit antibody was used to detect binding of anti-TIGIT to the mTIGIT-Fc-GITRL chimeric protein. This assay detects the intact TIGIT-Fc-GITRL chimeric protein because detection requires simultaneous binding to anti-GITRL and anti-TIGIT antibodies. The amount of the mTIGIT-Fc-GITRL chimeric protein from the time of injection in mice that received LNP comprising the modified mRNA encoding the mTIGIT-Fc-GITRL chimeric protein increased from 4 hours to 24 hr and to 48 hours (data not shown). A fold-change in serum concentrations of the TIGIT-Fc-GITRL chimeric protein from each group at 24 or 48 hours compared to the 4-hour time point is plotted. As shown in FIG. 3, the serum concentration of the TIGIT-Fc-GITRL chimeric protein decreased by about 50% in 24 hours in mice receiving 50 μg of purified recombinant TIGIT-Fc-GITRL chimeric protein and further decreased from 24 to 48 hours. On the other hand, the mice that received mRNA encoding the TIGIT-Fc-GITRL chimeric protein, exhibited an about 5-fold increase in the serum concentration of the TIGIT-Fc-GITRL chimeric protein (FIG. 3). The serum concentration of the TIGIT-Fc-GITRL chimeric protein further increased from 24 to 48 hours (FIG. 3).

These results demonstrate, inter alia, that delivery of a modified mRNA encoding the TIGIT-Fc-GITRL chimeric protein leads to the expression of the TIGIT-Fc-GITRL chimeric protein in vivo. These results also indicated, inter alia, that whereas the serum concentrations of TIGIT-Fc-GITRL in mice that were injected with purified recombinant TIGIT-Fc-GITRL chimeric protein underwent a rapid decline, which, without being bound by theory, may be indicative of TMDD (target mediated drug disposition), the serum concentrations of the TIGIT-Fc-GITRL chimeric protein mice that received LNP comprising the modified mRNA encoding the TIGIT-Fc-GITRL chimeric protein appear to accumulate over time (e.g., from 4 hr to 24 hr to 48 hours).

### Example 3: Tissue Accumulation of the TIGIT-Fc-GITRL Chimeric Protein after Modified mRNA-Based Delivery

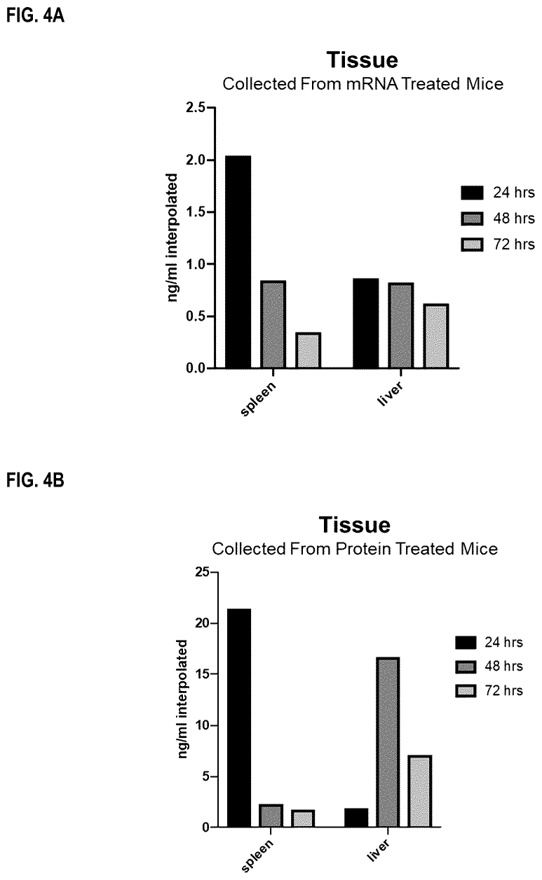

Mice were injected IV with 3.5 μg of TIGIT-Fc-LIGHT mRNA formulated with in vivo-jetRNA lipid-based delivery reagent (Polyplus) or 50 μg of the purified recombinant TIGIT-Fc-GITRL chimeric protein. After 24, 48, and 72 hours, spleens and livers were collected from some mice. The tissue samples were dissociated and the resulting slurry or the harvested serum were assessed by an MSD/ELISA assay that was designed specifically to detect both functional domains of mTIGIT-Fc-GITRL. Briefly, the MSD GOLD 96-well Streptavidin SECTOR plates were coated with biotinylated anti-mouse GITRL goat polyclonal antibody (R&D Systems #BAF2177) and increasing amounts of the cell culture supernatants were added to the plate for capture by the plate-bound anti-mouse GITRL goat polyclonal antibody. A sample spiked with 50 μg of recombinant mouse TIGIT-Fc-GITRL protein was included as a positive control. Binding of the TIGIT-Fc-GITRL chimeric protein to the anti-mouse GITRL goat polyclonal antibody was detected using anti-mouse TIGIT rabbit IgG recombinant mAb (R&D Systems #MAB72671). A secondary SULFO TAG anti-Rabbit antibody was used to detect binding of anti-TIGIT to the mTIGIT-Fc-GITRL chimeric protein. This assay detects the intact TIGIT-Fc-GITRL chimeric protein because detection requires simultaneous binding to anti-GITRL and anti-TIGIT antibodies. As shown in FIG. 4A, the TIGIT-Fc-GITRL chimeric protein could be detected both in spleen and in liver 24, 48, and 72 hours after the administration of the TIGIT-Fc-LIGHT mRNA formulated with in vivo-jetRNA lipid-based delivery reagent. The amount of the TIGIT-Fc-GITRL chimeric protein in liver was very stable and the amount of the TIGIT-Fc-GITRL chimeric protein in spleen decreased by about 60% from 24 to 48 hours and from 48 to 72 hours (FIG. 4A). on the other hand, the amount of the TIGIT-Fc-GITRL chimeric protein in spleen decreased to background levels in mice that received the purified recombinant TIGIT-Fc-GITRL chimeric protein (FIG. 4B). The amount of the purified recombinant TIGIT-Fc-GITRL chimeric protein in mice that received the purified recombinant TIGIT-Fc-GITRL chimeric protein decreased from 48 to 72 hr more than in the mice that received the TIGIT-Fc-LIGHT mRNA formulated with in vivo-jetRNA lipid-based delivery reagent (compare FIG. 4A with FIG. 4B).

These results demonstrate, inter alia, that a modified mRNA-based delivery of the TIGIT-Fc-GITRL chimeric protein causes accumulation of the TIGIT-Fc-GITRL chimeric protein in tissues. These results also indicated, inter alia, that the serum concentrations of the TIGIT-Fc-GITRL chimeric protein mice that received LNP comprising the modified mRNA encoding the TIGIT-Fc-GITRL chimeric protein appear to accumulate over time, whereas the serum concentrations of TIGIT-Fc-GITRL in mice that were injected with purified recombinant TIGIT-Fc-GITRL chimeric protein underwent a rapid decline, which, without being bound by theory, may be indicative of TMDD (target mediated drug disposition).

Collectively, these results demonstrate that mRNA/LNP delivery of bi-specific chimeric proteins results in their sustained expression in tissues.

### Example 4: In Vivo Expression of Serum Cytokines in Response to the Modified mRNA-Based Delivery of the TIGIT-Fc-GITRL Chimeric Protein

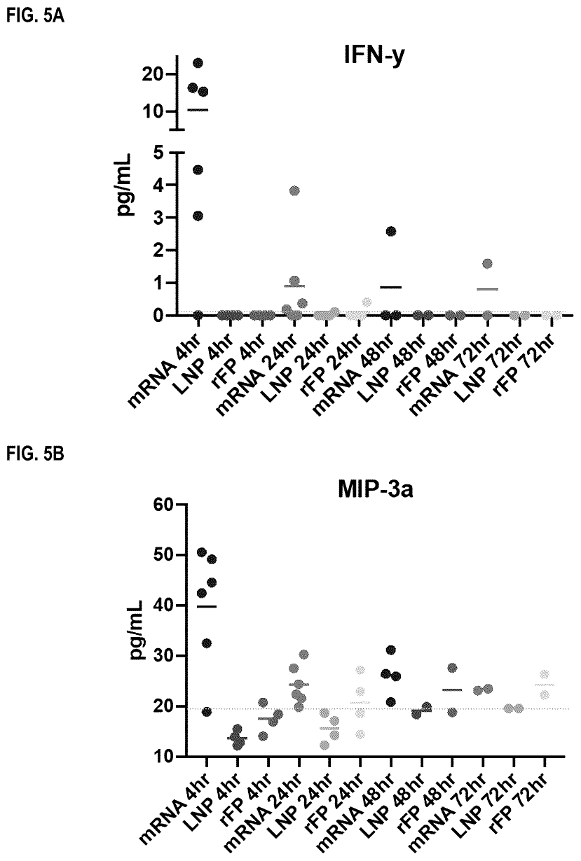

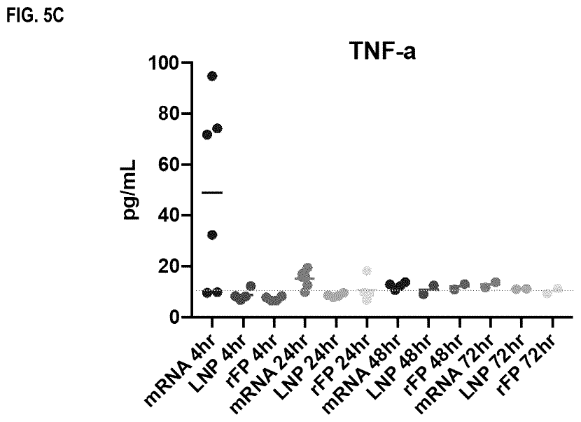

Mice were injected IV with 3.5 μg of TIGIT-Fc-LIGHT mRNA formulated with in vivo-jetRNA lipid-based delivery reagent (Polyplus), a control LNP that lacked mmRNA, or 50 μg of the purified recombinant TIGIT-Fc-GITRL chimeric protein. The empty LNP and the mouse TIGIT-Fc-GITRL chimeric protein served as negative control and positive control, respectively. After 4, 24, 48, and 72 hours, serum was collected from the mice, and IFNγ, MIP-3a (CCL20) and TNFα were assessed using the Meso Scale Discovery (MSD) ELISA assay format. The results are shown in FIG. 5A to FIG. 5C, where TIGIT-Fc-LIGHT mRNA is indicated as “mRNA,” empty LNP as “LNP,” and the TIGIT-Fc-GITRL chimeric protein as “fFP.” Dotted lines is drawn at the level of the highest signal achieved with the negative control (empty LNP). As shown in FIG. 5A, the mice treated with mRNA exhibited an induction of IFNγ compared to the empty LNP negative control at 4, 24, 48 and 72 hours, indicating a sustained biological activity. On the other hand, the TIGIT-Fc-GITRL chimeric protein exhibited an induction of IFNγ (FIG. 5A), which was consistent with the pharmacokinetics shown in FIG. 3. As shown in FIG. 5B, similarly, the mice treated with mRNA exhibited an induction of MIP-3a compared to the empty LNP negative control at 4, 24, 48 and 72 hours, indicating a sustained biological activity. The TIGIT-Fc-GITRL chimeric protein exhibited induction of MIP-3a, at 24 to 72 hours (FIG. 5B). Similarly, as shown in FIG. 5C, the mice treated with mRNA exhibited an induction of IFNγ compared to the empty LNP negative control at 4, 24, 48 and 72 hours, indicating a sustained biological activity. On the other hand, the TIGIT-Fc-GITRL chimeric protein exhibited an induction of IFNγ (FIG. 5C).

These results demonstrate, inter alia, that a modified mRNA-based delivery of the TIGIT-Fc-GITRL chimeric protein induces sustained production of adaptive and innate immune cytokines (e.g., IFNγ, MIP3a, and TNFα).

### Example 5: In Vivo Margination of Target Immune Cell Populations

Mice were randomly divided in the following seven groups and subjected to the following were administered intravenously: (1) vehicle only control (LNP that lacked mmRNA), (2) 200 μg of recombinant mouse (rm) SIRPα-Fc-CD40L chimeric protein (“rm SIRPα-Fc-CD40L”), (3) 12.5 μg of mRNA encoding the SIRPα-Fc-CD40L chimeric protein formulated with in vivo-jetRNA lipid-based delivery reagent (Polyplus; “12.5 μg SIRPα-Fc-CD40L mRNA”), (4) 25 μg of mRNA encoding the SIRPα-Fc-CD40L chimeric protein formulated with in vivo-jetRNA lipid-based delivery reagent (Polyplus; “25 μg SIRPα-Fc-CD40L mRNA”), (5) 200 μg of recombinant mouse (rm) TIGIT-Fc-LIGHT chimeric protein (“rm TIGIT-Fc-LIGHT”), (6) 12.5 μg of mRNA encoding the TIGIT-Fc-LIGHT chimeric protein formulated with in vivo-jetRNA lipid-based delivery reagent (Polyplus; “12.5 μg TIGIT-Fc-LIGHT mRNA”), and (7) 25 μg of mRNA encoding the TIGIT-Fc-LIGHT chimeric protein formulated with in vivo-jetRNA lipid-based delivery reagent (Polyplus; “25 μg TIGIT-Fc-LIGHT mRNA”). After 3 and 24 hours, blood samples were collected from the mice, and the abundance of total CD8+ cells compared to total CD45+CD3+ cells, CD8+CD69+ T cells compared to total CD45+CD3+ cells, total NKP46+ Natural Killer cells (NK cells) compared to total CD45+CD3− cells.

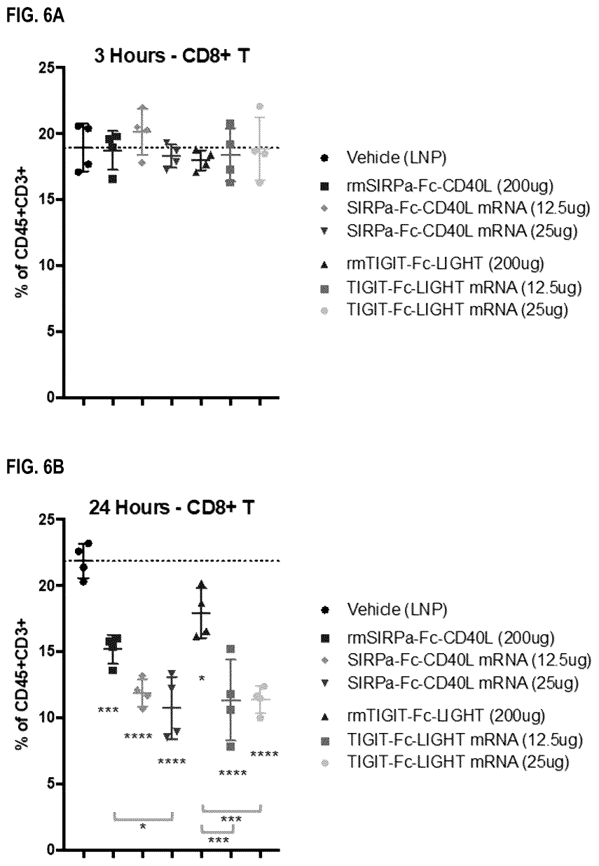

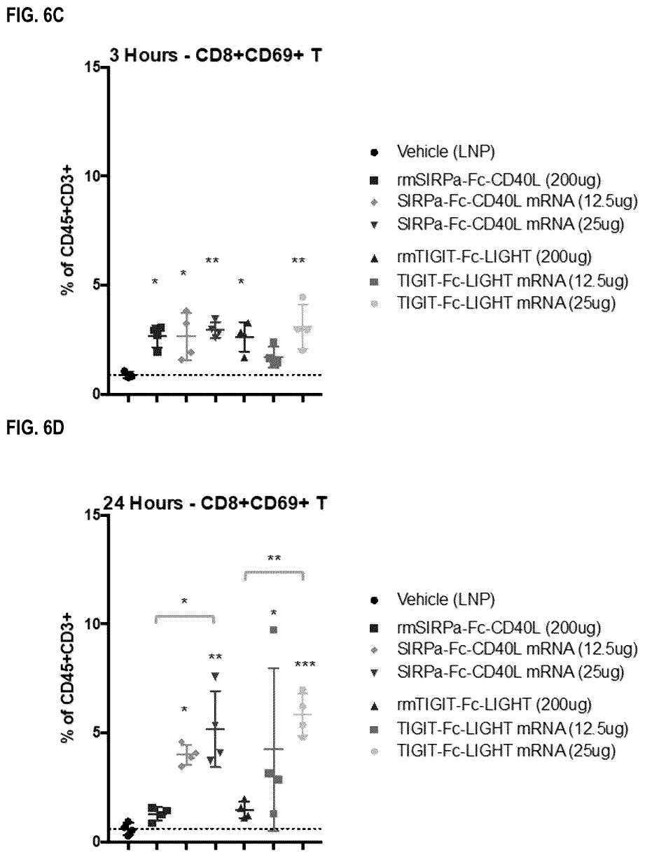

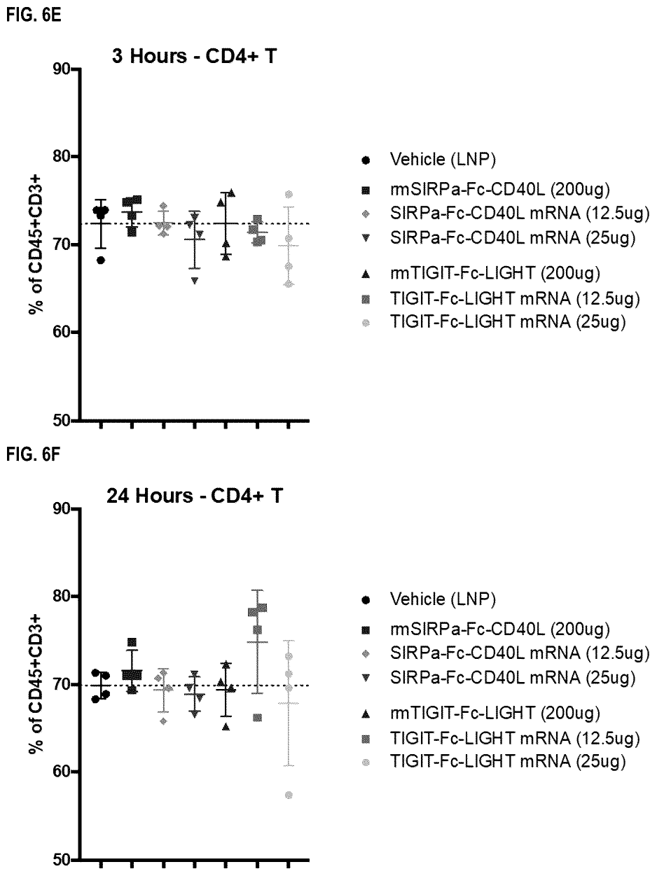

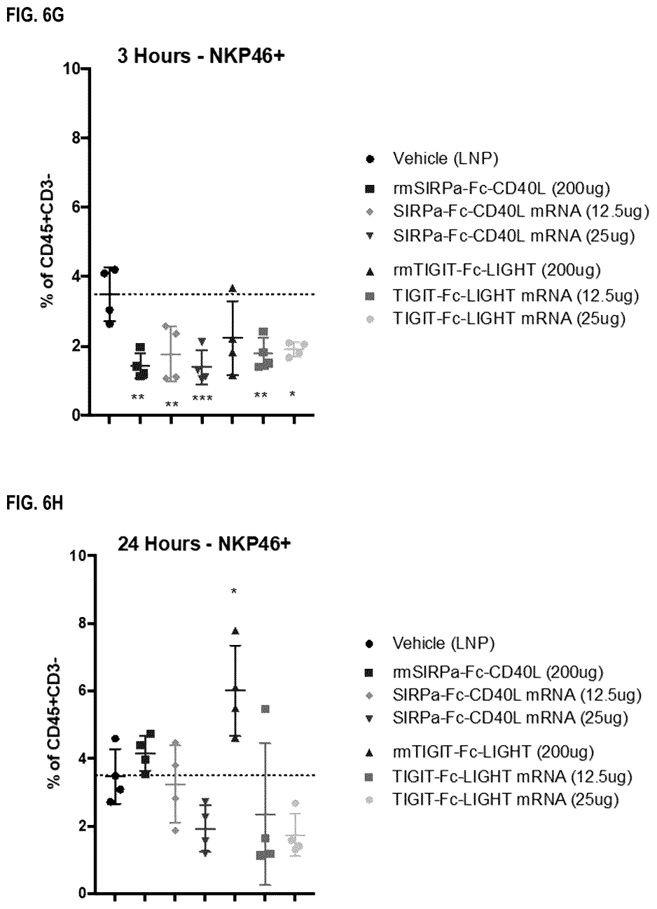

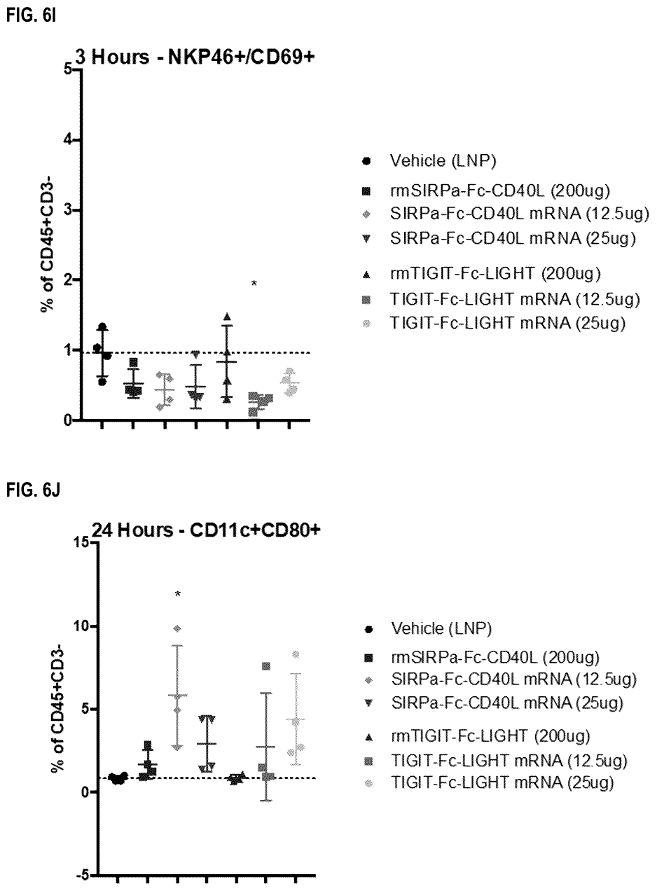

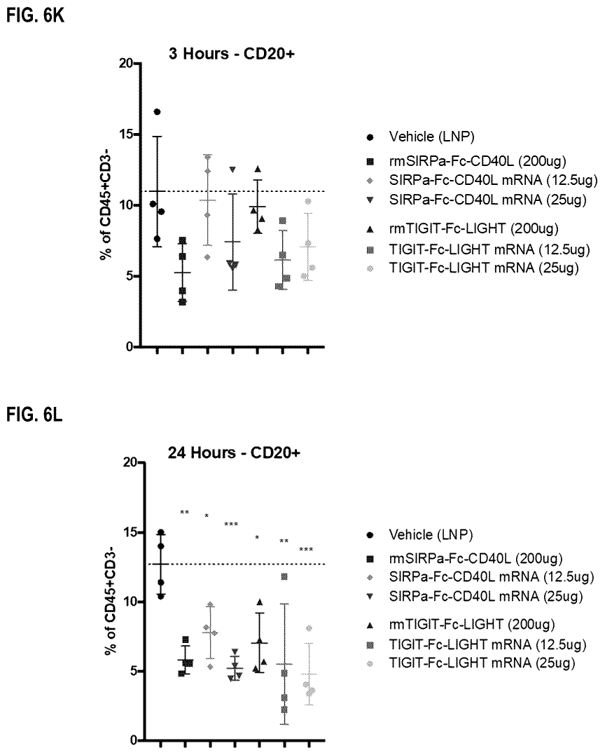

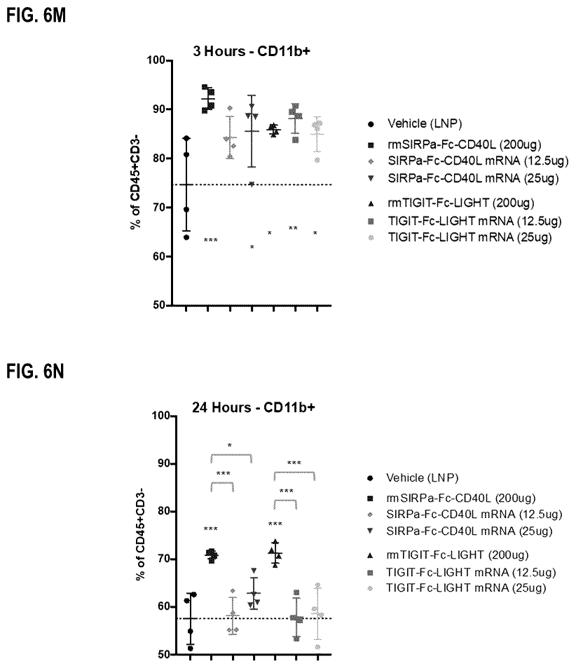

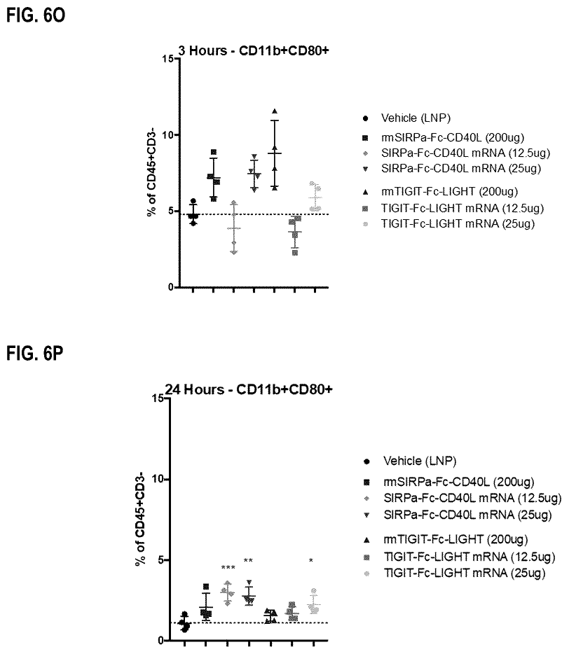

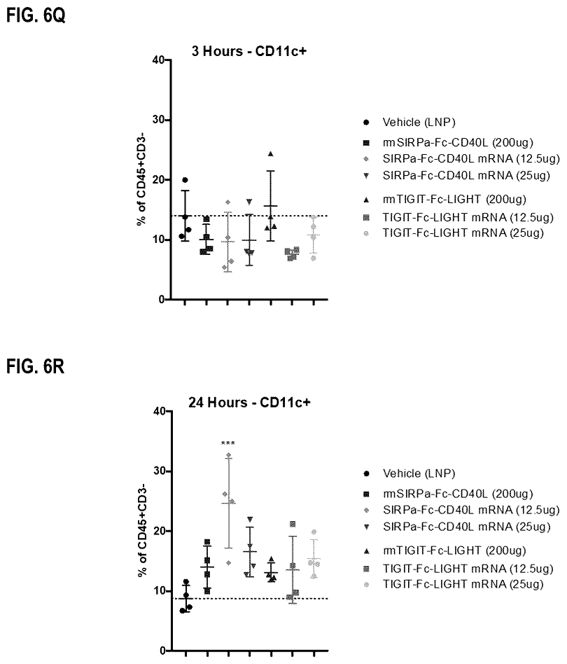

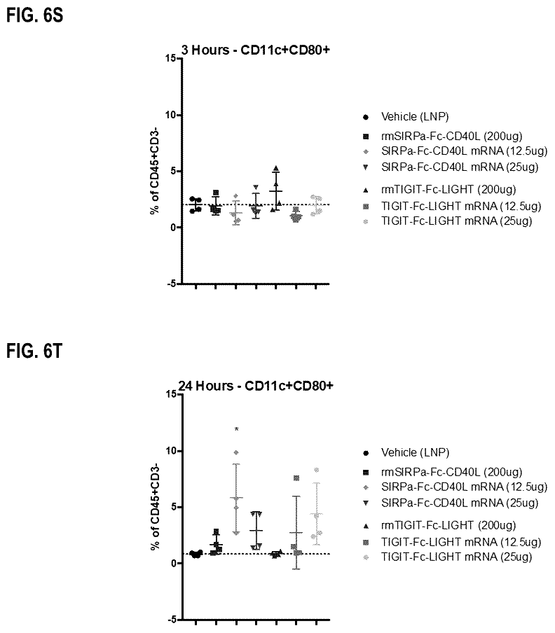

As shown in FIG. 6A, the abundance of total CD8+ cells compared to total CD45+CD3+ cells remained substantially unchanged at 3 hours post-dosing. Interestingly, the abundance of total CD8+ cells compared to total CD45+CD3+ cells significantly reduced at 24 hours post-dosing following the treatment with each of rm SIRPα-Fc-CD40L (p<0.001), 12.5 μg SIRPα-Fc-CD40L mRNA (p<0.0001), 25 μg SIRPα-Fc-CD40L mRNA (p<0.0001), rm TIGIT-Fc-LIGHT (p<0.05), 12.5 μg TIGIT-Fc-LIGHT mRNA (p<0.0001), and 25 μg TIGIT-Fc-LIGHT mRNA (p<0.0001) (FIG. 6B). This illustrates margination of CD8+ lymphocytes. Interestingly, 25 μg SIRPα-Fc-CD40L mRNA produced a significantly more CD8+ lymphocyte margination at 24 hours post-dosing compared to rm SIRPα-Fc-CD40L (p<0.05) (FIG. 6B). Similarly, 12.5 μg TIGIT-Fc-LIGHT mRNA (p<0.001) and 25 μg TIGIT-Fc-LIGHT mRNA (p<0.001) produced significantly more CD8+ lymphocyte margination at 24 hours post-dosing compared to rm TIGIT-Fc-LIGHT (p<0.001). This is consistent with the observed accumulation of the chimeric proteins following nucleic acid-based delivery (FIG. 3).

Next, the effect of these treatments on activated CD8+CD69+ T cells was measured. As shown in FIG. 6C, activated CD8+CD69+ T cells significantly increased compared to total CD45+CD3+ cells at 3 hours post-dosing following the treatment with each of rm SIRPα-Fc-CD40L (p<0.05), 12.5 μg SIRPα-Fc-CD40L mRNA (p<0.05), 25 μg SIRPα-Fc-CD40L mRNA (p<0.01), rm TIGIT-Fc-LIGHT (p<0.05), 12.5 μg TIGIT-Fc-LIGHT mRNA, and 25 μg TIGIT-Fc-LIGHT mRNA (p<0.01) (FIG. 6C). This illustrates activation of CD8+ lymphocytes, which is consistent with the observed cytokine production induced by the chimeric proteins (FIGS. 5A-5C). As shown in FIG. 6D, the abundance of activated peripheral CD8+CD69+ T cells remained significantly increased compared to total CD45+CD3+ cells at 24 hours post-dosing following the treatment with 12.5 μg SIRPα-Fc-CD40L mRNA (p<0.05), 25 μg SIRPα-Fc-CD40L mRNA (p<0.01), 12.5 μg TIGIT-Fc-LIGHT mRNA (p<0.05), and 25 μg TIGIT-Fc-LIGHT mRNA (p<0.001) (FIG. 6C). Interestingly, 25 μg SIRPα-Fc-CD40L mRNA produced a significantly the abundance of activated peripheral CD8+CD69+ T cells compared to rm SIRPα-Fc-CD40L (p<0.05) (FIG. 6D). Similarly, 25 μg TIGIT-Fc-LIGHT mRNA (p<0.001) produced significantly more abundance of activated peripheral CD8+CD69+ T cells compared to rm TIGIT-Fc-LIGHT (p<0.01).

As expected, minimal change in CD4+ T cells compared to total CD45+CD3+ cells at 3 hours (FIG. 6E) and 24 hours post-dosing (FIG. 6F) with each of the treatment.

Next, it was observed that these treatments on the margination of total NKP46+ natural killer cells at 3 hours post-dosing (FIG. 6G). As shown in FIG. 6H, the margination of total NKP46+ natural killer cells remained significantly increased compared to total CD45+CD3+ cells at 24 hours post-dosing following the treatment with rm SIRPα-Fc-CD40L compared to that in 12.5 μg SIRPα-Fc-CD40L mRNA, 25 μg SIRPα-Fc-CD40L mRNA. Similarly, the margination of total NKP46+ natural killer cells remained significantly increased compared to total CD45+CD3+ cells at 24 hours post-dosing following the treatment with rm TIGIT-Fc-LIGHT compared to 12.5 μg TIGIT-Fc-LIGHT mRNA, and 25 μg TIGIT-Fc-LIGHT mRNA (FIG. 6H). An enrichment of activated CD69+ NKP46+ cells was observed at 24 hours post-dosing (FIG. 6J) but not 3 hours post-dosing (FIG. 6I) and 24 hours).

Similarly, margination of CD20+ B Cells compared to total CD45+CD3− cells was observed at 24 hours post-dosing (FIG. 6L), and to a lesser degree at 3 hours (FIG. 6K).

A modest increase in total CD11b+ Antigen Presenting Cells (APCs) was observed at 3 hours post-dosing (FIG. 6M) and 24 hours post-dosing (FIG. 6N). Similarly, a modest increase in activated CD11b+CD80+ APCs compared to total CD45+CD3− cells was observed at 3 hours (FIG. 6O) and 24 hours post-dosing (FIG. 6P). A modest increase in total CD11c+ APCs compared to total CD45+CD3− cells was observed at 3 hours (FIG. 6Q) and 24 hours post-dosing (FIG. 6R). A modest increase in activated CD11c+CD80+ APCs was observed compared to total CD45+CD3− cells at 3 hours (FIG. 6S) and 24 hours post-dosing (FIG. 6T).

Collectively, these results demonstrate, inter alia, that mRNA encoding the present chimeric proteins resulted in equivalent or greater target immune cell margination than the recombinant proteins. Without wishing to be bound by theory, the enrichment of activated immune cells in the peripheral blood could be reflective of differing kinetics of margination, activation, proliferation, and return to the circulation.

### Example 6: Sustained Immune Activation Induced by mmRNA Encoding the SIRPα-Fc-CD40L or TIGIT-Fc-LIGHT Chimeric Proteins

The in vivo changes in activated lymphocytes induced by LNP comprising 12.5 μg or 25 μg of modified mRNA (mmRNA) encoding the SIRPα-Fc-CD40L or TIGIT-Fc-LIGHT chimeric proteins was tracked for an extended time. Briefly, 1×10⁶ tumor cells were injected subcutaneously into the lower flanks of mice. When tumors became were palpable, the mice were randomly distributed in seven treatment groups and treated as follows:
Group 1: empty LNP lacking mmRNA,
Group 2: 200 μg of the purified recombinant mouse SIRPα-Fc-CD40L chimeric protein,
Group 3: LNP comprising 12.5 μg mmRNA encoding the mouse SIRPα-Fc-CD40L chimeric protein,
Group 4: LNP comprising 25 μg mmRNA encoding the mouse SIRPα-Fc-CD40L chimeric protein,
Group 5: 200 μg of the purified recombinant mouse TIGIT-Fc-LIGHT chimeric protein,
Group 6: LNP comprising 12.5 μg mmRNA encoding the mouse TIGIT-Fc-LIGHT chimeric protein, and
Group 7: LNP comprising 25 μg mmRNA encoding the mouse TIGIT-Fc-LIGHT chimeric protein.

Tumor-draining lymph nodes (TDLN) and tumors were recovered from the mice at 24 hr, 48 hr, 72 hr and 168 hr post-treatment. The tissue samples were dissociated and CD3+ cells were enriched from the dissociated tissue samples. The CD3+-enriched cell population was stained with anti-CD8, anti-CD69, anti-CD45 and anti-CD3 antibodies and subjected to flow cytometry. The percentage of activated CD8+CD69+ T cells (out of CD45+CD3+) in CD3+-enriched dissociated TDLN and tumor samples were determined.

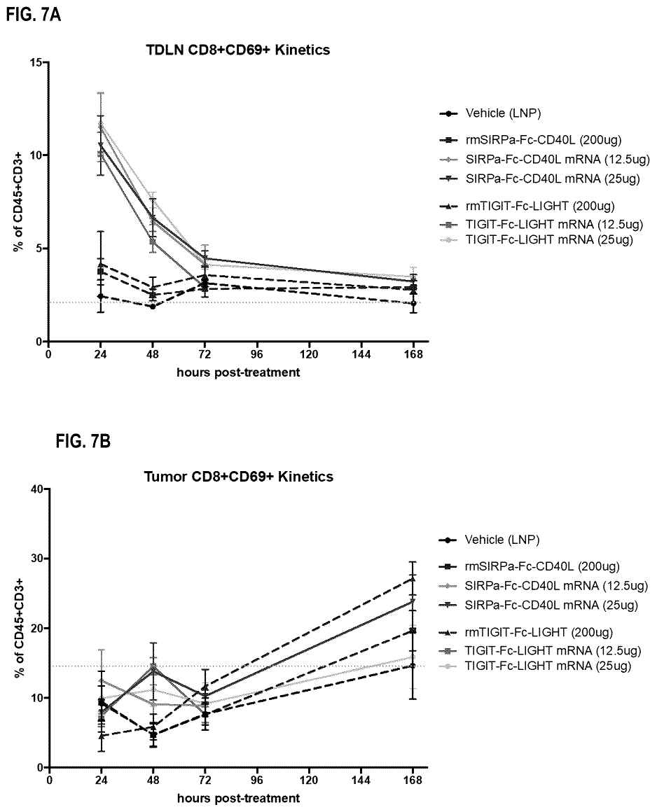

As shown in FIG. 7A, TDLN of the mice that were treated with the purified SIRPα-Fc-CD40L or TIGIT-Fc-LIGHT chimeric proteins showed an about 1.5-1.8 fold increased levels of activated CD8+CD69+ T cells (and NK cells) at 24 hr post-treatment compared to the mice that received vehicle only (empty LNP). On the other hand, TDLN of the mice treated with LNP comprising mmRNA encoding the SIRPα-Fc-CD40L or TIGIT-Fc-LIGHT chimeric proteins showed 4 to 4.5 fold increased levels of activated CD8+CD69+ T cells (and NK cells) at 24 hr post-treatment compared to the mice that received vehicle only (FIG. 7A). Interestingly, TDLN of the mice treated with LNP comprising mmRNA encoding the SIRPα-Fc-CD40L or TIGIT-Fc-LIGHT chimeric proteins showed 2.5 to 3 fold higher levels of activated CD8+CD69+ T cells (and NK cells) at 24 hr post-treatment compared to the mice that received purified SIRPα-Fc-CD40L or TIGIT-Fc-LIGHT chimeric proteins (FIG. 7A). At 48 hr post-treatment, the TDLN of the mice treated with LNP comprising mmRNA encoding the SIRPα-Fc-CD40L or TIGIT-Fc-LIGHT chimeric proteins continued to show higher levels of activated CD8+CD69+ T cells (and NK cells) compared to the mice that received either vehicle only or purified SIRPα-Fc-CD40L or TIGIT-Fc-LIGHT chimeric proteins (FIG. 7A). While the difference diminished at 72 and 168 hours, the curves for the mice that received LNP comprising 25 μg mmRNA encoding the SIRPα-Fc-CD40L or TIGIT-Fc-LIGHT chimeric proteins remained above the rest of the curves, indicating higher levels of activated CD8+CD69+ T cells (and NK cells) even at 72 or 168 hr post-treatment). As shown in FIG. 7B, all treatment groups showed higher levels of activated CD8+CD69+ T cells (and NK cells compared to the vehicle-alone control.

These results indicate, inter alia, rapid and sustained enrichment of activated immune cells (e.g., CD8+CD69+ T cells and NK cells) following the administration of mmRNA encoding the chimeric proteins of the present disclosure.

### Example 7: In Vivo Efficacy of mmRNA Encoding the SIRPα-Fc-CD40L or TIGIT-Fc-LIGHT Chimeric Proteins

The anti-tumor efficacy of modified mRNA (mmRNA) encoding the SIRPα-Fc-CD40L or TIGIT-Fc-LIGHT chimeric proteins was studied in comparison with untreated mice, vehicle-alone treated mice and mice treated with the purified SIRPα-Fc-CD40L or TIGIT-Fc-LIGHT chimeric proteins. Briefly, 1×10⁶ tumor cells were injected subcutaneously into the lower flanks of mice. When tumors became were palpable, the mice were randomly distributed in eight treatment groups and treated as follows:
Group 1: Untreated mice,
Group 2: empty LNP lacking mmRNA,
Group 3: 200 μg of the purified recombinant mouse SIRPα-Fc-CD40L chimeric protein,
Group 4: LNP comprising 12.5 μg mmRNA encoding the mouse SIRPα-Fc-CD40L chimeric protein,
Group 5: LNP comprising 25 μg mmRNA encoding the mouse SIRPα-Fc-CD40L chimeric protein,
Group 6: 200 μg of the purified recombinant mouse TIGIT-Fc-LIGHT chimeric protein,
Group 7: LNP comprising 12.5 μg mmRNA encoding the mouse TIGIT-Fc-LIGHT chimeric protein, and
Group 8: LNP comprising 25 μg mmRNA encoding the mouse TIGIT-Fc-LIGHT chimeric protein.

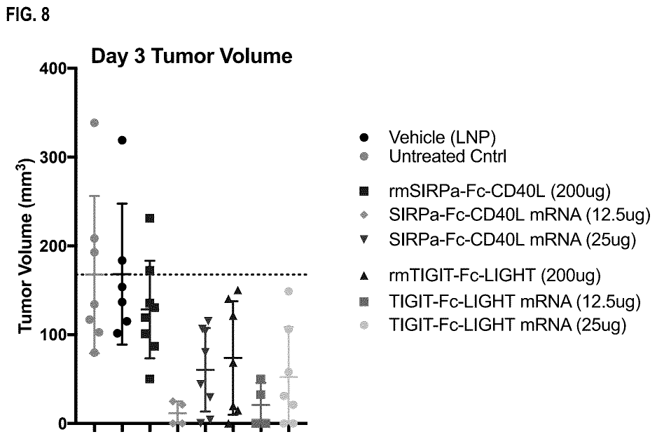

Tumors were measured with electronic calipers on day 3 and plotted using the GraphPad Prism software. Dotted lines were drawn at the mean of the vehicle alone-treated group (Group 2). As shown in FIG. 8, the treatments with the purified SIRPα-Fc-CD40L or TIGIT-Fc-LIGHT chimeric proteins showed a decrease in tumor volume, compared to both untreated mice, and vehicle-alone-treated mice. The mice treated with LNP comprising mmRNA encoding the SIRPα-Fc-CD40L or TIGIT-Fc-LIGHT chimeric proteins showed a decrease in tumor volume, compared to both untreated mice, and vehicle-alone-treated mice (FIG. 8). Interestingly, the mice treated with LNP comprising mmRNA encoding the SIRPα-Fc-CD40L or TIGIT-Fc-LIGHT chimeric proteins showed a decrease in tumor volume, compared to the purified SIRPα-Fc-CD40L or TIGIT-Fc-LIGHT chimeric proteins.

These results indicate, inter alia, that mmRNA encoding the chimeric proteins of the present disclosure may be beneficial in the treatment of cancer, and thus, may be useful in the methods disclosed herein. These results further indicate, inter alia, that rapid and sustained immune stimulation induced by the mmRNA encoding the chimeric proteins of the present disclosure results in immediate control of tumor growth.

### Example 8: In Vivo Persistence of Modified mRNA (mmRNA) Encoding the mmRNA Encoding the SIRPα-Fc-CD40L Chimeric Protein

The in vivo persistence of modified mRNA (mmRNA) encoding the SIRPα-Fc-CD40L chimeric protein was tracked for an extended time in tumor-bearing mice. Briefly, 1×10⁶ tumor cells were injected subcutaneously into the lower flanks of mice. When tumors became were palpable, the mice were randomly distributed in three treatment groups and treated as follows:
Group 1: empty LNP lacking mmRNA,
Group 2: 200 μg of the purified recombinant mouse SIRPα-Fc-CD40L chimeric protein, and
Group 3: LNP comprising 12.5 μg mmRNA encoding the mouse SIRPα-Fc-CD40L chimeric protein.

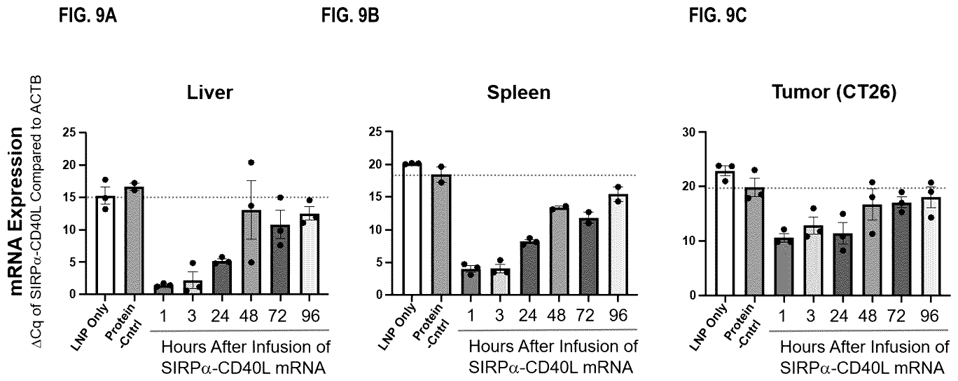

Mice that were administered empty LNP or the SIRPα-Fc-CD40L chimeric protein were sacrificed atone hour post administration and their livers, spleens and tumors were extracted. The mice that were administered LNP comprising mmRNA encoding the SIRPα-Fc-CD40L chimeric protein were sacrificed at 1, 3, 24, 48, 72, or 96 hours post administration, and their livers, spleens and tumors were extracted. RNA was isolated from liver, spleen and tumor tissues and subjected to reverse transcription-polymerase chain reaction (RT-PCR) using primers that span the SIRPα and Fc domain (therefore specific to the SIRPα-Fc-CD40L chimeric protein) and analyzed using the ΔCq method using beta actin (ACTB) primers, which were used as a house-keeping gene control. The difference between Cq values obtained with the ACTB primers and the SIRPα-Fc-CD40L primers were determined for each sample. Lower ΔCq values indicate greater mRNA levels in these assays. The results obtained with liver (FIG. 9A), spleen (FIG. 9B) and tumor (FIG. 9C) samples at various time point were plotted. The dotted line shows the ΔCq of SIRPα-Fc-CD40L mmRNA compared to ACTB mRNA in mice that received empty LNP.

As shown in FIG. 9A, livers of the mice that were administered LNP comprising the mmRNA exhibited high levels of the mmRNA at 1 hour-post dosing compared to the livers of mice that received empty LNP or the purified recombinant protein. The levels of mmRNA in liver remained high at 3 and 24 hours and remained detectable at 48, 72 and 96 hours compared to the livers of mice that received empty LNP or the purified recombinant protein (FIG. 9A).

As shown in FIG. 9B, spleens of the mice that were administered LNP comprising the mmRNA exhibited high levels of the mmRNA at 1 hour-post dosing compared to the spleens of mice that received empty LNP or the purified recombinant protein. The levels of mmRNA in spleen remained high at 3 and 24 hours and remained detectable at 48, 72 and 96 hours compared to the spleens of mice that received empty LNP or the purified recombinant protein (FIG. 9B).

As shown in FIG. 9C, tumors of the mice that were administered LNP comprising the mmRNA exhibited high levels of the mmRNA, albeit lower compared to liver or spleen, at 1 hour-post dosing compared to the tumors of mice that received empty LNP or the purified recombinant protein. The levels of mmRNA in tumor remained high at 3 and 24 hours and remained detectable at 48, 72 and 96 hours compared to the tumors of mice that received empty LNP or the purified recombinant protein (FIG. 9C).

These results demonstrate, inter alia, that the mmRNA persists for days after administration in tissues such as liver, spleen and tumor.

### Example 9: In Vivo Persistence of Modified mRNA (mmRNA) Encoding the mmRNA Encoding the TIGIT-Fc-LIGHT Chimeric Protein

The in vivo persistence of modified mRNA (mmRNA) encoding the TIGIT-Fc-LIGHT chimeric protein was tracked for an extended time in tumor-bearing mice. Briefly, 1×10⁶ tumor cells were injected subcutaneously into the lower flanks of mice. When tumors became were palpable, the mice were randomly distributed in three treatment groups and treated as follows:
Group 1: empty LNP lacking mmRNA,
Group 2: 200 μg of the purified recombinant mouse TIGIT-Fc-LIGHT chimeric protein, and
Group 3: LNP comprising 12.5 μg mmRNA encoding the mouse TIGIT-Fc-LIGHT chimeric protein.

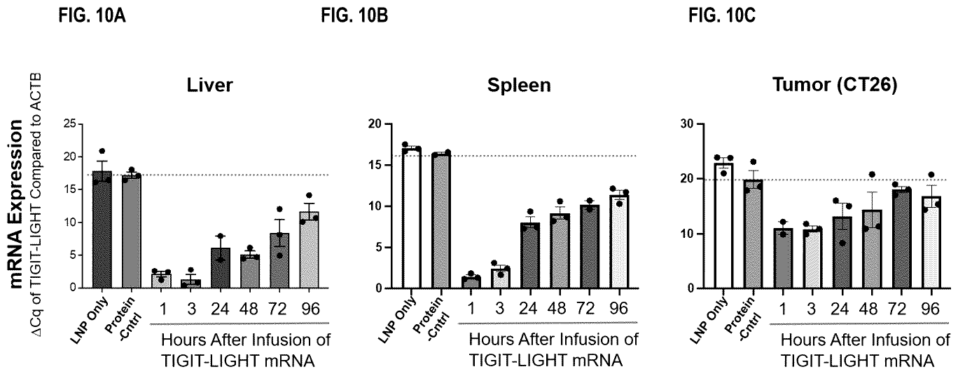

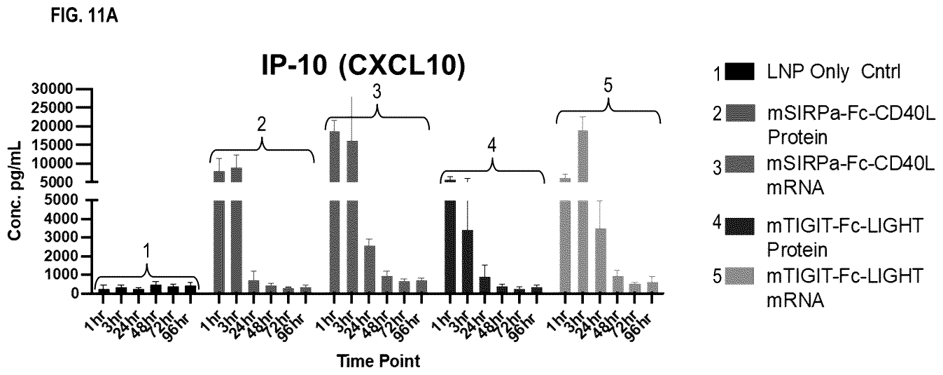

Mice that were administered empty LNP or the TIGIT-Fc-LIGHT chimeric protein were sacrificed at one hour post administration and their livers, spleens and tumors were extracted. The mice that were administered LNP comprising mmRNA encoding the TIGIT-Fc-LIGHT chimeric protein were sacrificed at 1, 3, 24, 48, 72, or 96 hours post administration, and their livers, spleens and tumors were extracted. RNA was isolated from liver, spleen and tumor tissues and subjected to RT-PCR using primers that span the TIGIT and Fc domain (therefore specific to the TIGIT-Fc-LIGHT chimeric protein) and analyzed using the ΔCq method using beta actin (ACTB) primers, which were used as a house-keeping gene control. The difference between Cq values obtained with the ACTB primers and the TIGIT-Fc-LIGHT primers were determined for each sample. The results obtained with liver (FIG. 10A), spleen (FIG. 10B) and tumor (FIG. 10C) samples at various time point were plotted. The dotted line shows the ΔCq of TIGIT-Fc-LIGHT mmRNA compared to ACTB mRNA in mice that received empty LNP.

As shown in FIG. 10A, livers of the mice that were administered LNP comprising the mmRNA exhibited high levels of the mmRNA at 1 hour-post dosing compared to the livers of mice that received empty LNP or the purified recombinant protein. The levels of mmRNA in liver remained high at 3 and 24 hours and remained detectable at 48, 72 and 96 hours compared to the livers of mice that received empty LNP or the purified recombinant protein (FIG. 10A). As shown in FIG. 10B, spleens of the mice that were administered LNP comprising the mmRNA exhibited high levels of the mmRNA at 1 hour-post dosing compared to the spleens of mice that received empty LNP or the purified recombinant protein. The levels of mmRNA in spleen remained high at 3 and 24 hours and remained detectable at 48, 72 and 96 hours compared to the spleens of mice that received empty LNP or the purified recombinant protein (FIG. 10B). As shown in FIG. 10C, tumors of the mice that were administered LNP comprising the mmRNA exhibited high levels of the mmRNA, albeit lower compared to liver or spleen, at 1 hour-post dosing compared to the tumors of mice that received empty LNP or the purified recombinant protein. The levels of mmRNA in tumor remained high at 3 and 24 hours and remained detectable at 48, 72 and 96 hours compared to the tumors of mice that received empty LNP or the purified recombinant protein (FIG. 10C).

These results demonstrate, inter alia, that the mmRNA persists for days after administration in tissues such as liver, spleen and tumor.

### Example 10: Sustained Cytokine Response Induced by mmRNA Encoding the SIRPα-Fc-CD40L or TIGIT-Fc-LIGHT Chimeric Proteins

Cytokine response induced by LNP comprising 12.5 μg or 25 μg of modified mRNA (mmRNA) encoding the SIRPα-Fc-CD40L or TIGIT-Fc-LIGHT chimeric proteins was studied. Briefly, 1×10⁶ tumor cells were injected subcutaneously into the lower flanks of mice. When tumors became were palpable, the mice were randomly distributed in five treatment groups and treated as follows:
Group 1: empty LNP lacking mmRNA,
Group 2: 200 μg of the purified recombinant mouse SIRPα-Fc-CD40L chimeric protein,
Group 3: LNP comprising 12.5 μg mmRNA encoding the mouse SIRPα-Fc-CD40L chimeric protein,
Group 4: 200 μg of the purified recombinant mouse TIGIT-Fc-LIGHT chimeric protein, and
Group 5: LNP comprising 12.5 μg mmRNA encoding the mouse TIGIT-Fc-LIGHT chimeric protein.

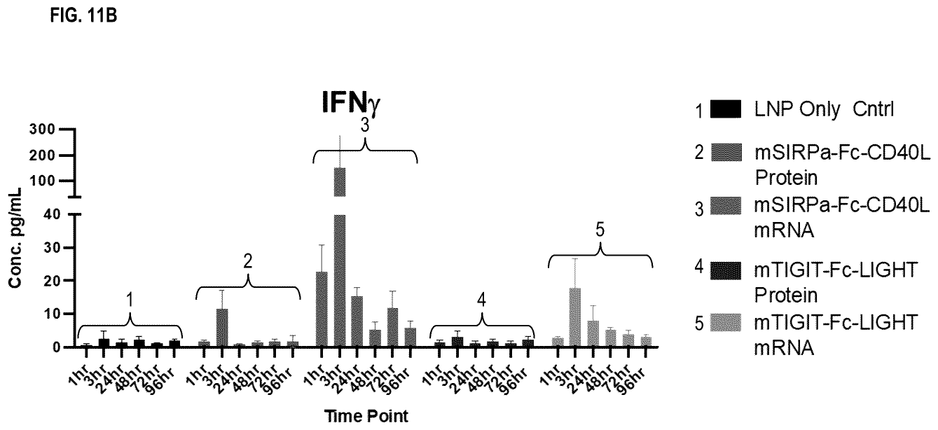

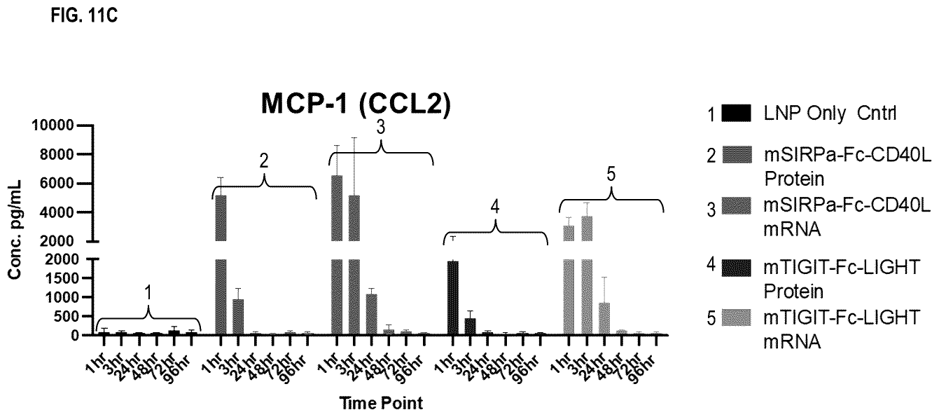

Blood was collected from the mice at 1 hr, 3 hr, 24 hr, 48 hr, and 72 hr-post dosing and the average levels of IP-10 (CXCL10), IFNγ, and MCP-1 (CCL2) were quantitated in these serum samples using ELISA-based assays. The results are plotted in FIG. 11A to FIG. 11C. As expected, IP-10 (CXCL10) (FIG. 11A), IFNγ (FIG. 11B), and MCP-1 (CCL2) (FIG. 11C) remained at background levels at all time points. As shown in FIG. 11A, the average levels of IP-10 (CXCL10) reached >5,000 pg/ml at 1 hour post-dosing in mice treated with the purified SIRPα-Fc-CD40L or TIGIT-Fc-LIGHT chimeric proteins. At 3 hours post-dosing, the average levels of IP-10 (CXCL10) remained high, >5,000 pg/ml and 3,000 pg/ml in mice treated with the purified SIRPα-Fc-CD40L or TIGIT-Fc-LIGHT chimeric proteins, respectively (FIG. 11A). However, as shown in FIG. 11A, the average levels of IP-10 (CXCL10) dropped to less than 1,000 pg/ml, and further decreased to background levels in mice treated with the purified SIRPα-Fc-CD40L or TIGIT-Fc-LIGHT chimeric proteins (FIG. 11A). As shown in FIG. 11A, the average levels of IP-10 (CXCL10) reached >5,000 pg/ml at 1 hour post-dosing in mice treated with the mmRNA encoding SIRPα-Fc-CD40L or TIGIT-Fc-LIGHT chimeric proteins. At 3 hours post-dosing, the average levels of IP-10 (CXCL10) reached >15,000 pg/ml in mice treated with the mmRNA encoding SIRPα-Fc-CD40L or TIGIT-Fc-LIGHT chimeric proteins (FIG. 11A). These levels were higher than the average levels of IP-10 (CXCL10) at 3 hours in mice treated with the purified SIRPα-Fc-CD40L or TIGIT-Fc-LIGHT chimeric proteins (FIG. 11A). Interestingly, as shown in FIG. 11A, at 24 hours, the average levels of IP-10 (CXCL10) remained >2,500 pg/ml in mice treated with the mmRNA encoding SIRPα-Fc-CD40L or TIGIT-Fc-LIGHT chimeric proteins, which were higher than the average levels of IP-10 (CXCL10) in mice treated with the purified SIRPα-Fc-CD40L or TIGIT-Fc-LIGHT chimeric proteins (FIG. 11A). Surprisingly, the average levels of IP-10 (CXCL10) at 48 hours remained about 1,000 pg/ml, and above background levels at 72 and 96 hours in mice treated with the mmRNA encoding SIRPα-Fc-CD40L or TIGIT-Fc-LIGHT chimeric proteins (FIG. 11A), in contrast to the average levels of IP-10 (CXCL10) in mice treated with the purified SIRPα-Fc-CD40L or TIGIT-Fc-LIGHT chimeric proteins.

The average levels of IFNγ were at background levels at 1 hour-post treatment in mice treated with the purified SIRPα-Fc-CD40L chimeric protein (FIG. 11B). As shown in FIG. 11B, the average levels of IFNγ reached >10 pg/ml at 3 hour post-dosing in mice treated with the purified SIRPα-Fc-CD40L chimeric protein. The average levels of IFNγ decreased to background levels at 24, 48, 72 and 96 hours-post treatment in mice treated with the purified SIRPα-Fc-CD40L chimeric protein (FIG. 11B). On the other hand, as shown in FIG. 11B, the average levels of IFNγ reached >20 pg/ml at 1 hour post-dosing in mice treated with the mmRNA encoding SIRPα-Fc-CD40L chimeric protein. At 3 hours post-dosing, the average levels of IFNγ reached about 150 pg/ml in mice treated with the mmRNA encoding SIRPα-Fc-CD40L chimeric protein (FIG. 11B). These levels were higher than the average levels of IFNγ at 3 hours in mice treated with the purified SIRPα-Fc-CD40L chimeric protein at 1 or 3 hours post-dosing (FIG. 11B). Interestingly, as shown in FIG. 11B, at 24 hours, the average levels of IFNγ remained >15 pg/ml in mice treated with the mmRNA encoding SIRPα-Fc-CD40L chimeric protein, which were higher than the average levels of IFNγ in mice treated with the purified SIRPα-Fc-CD40L chimeric protein (FIG. 11B). Surprisingly, the average levels of IFNγ at 48 hours remained above background levels at 48, 72 and 96 hours in mice treated with the mmRNA encoding SIRPα-Fc-CD40L chimeric proteins (FIG. 11B), in contrast to the average levels of IFNγ in mice treated with the purified SIRPα-Fc-CD40L chimeric protein.

Although there may be a peak at around 3 hours port-treatment, the average levels of IFNγ remained at background levels at 1 hour-post treatment in mice treated with the purified TIGIT-Fc-LIGHT chimeric protein at all time points tested (FIG. 11B). On the other hand, as shown in FIG. 11B, the average levels of IFNγ reached >15 pg/ml at 3 hour post-dosing in mice treated with the mmRNA encoding TIGIT-Fc-LIGHT chimeric protein. These levels were higher than the average levels of IFNγ at 3 hours in mice treated with the purified TIGIT-Fc-LIGHT chimeric protein at 1 or 3 hours post-dosing (FIG. 11B). Interestingly, as shown in FIG. 11B, at 24 hours, the average levels of IFNγ remained about 8 pg/ml in mice treated with the mmRNA encoding TIGIT-Fc-LIGHT chimeric protein, which were higher than the average levels of IFNγ in mice treated with the purified TIGIT-Fc-LIGHT chimeric protein (FIG. 11B). Surprisingly, the average levels of IFNγ at 48 hours remained above background levels at 48, 72 and 96 hours in mice treated with the mmRNA encoding TIGIT-Fc-LIGHT chimeric proteins (FIG. 11B), in contrast to the average levels of IFNγ in mice treated with the purified TIGIT-Fc-LIGHT chimeric protein.

As shown in FIG. 11C, the average levels of MCP-1 (CCL2) reached >5,000 pg/ml and about 1,000 pg/ml at 1 hour post-dosing in mice treated with the purified SIRPα-Fc-CD40L or TIGIT-Fc-LIGHT chimeric proteins, respectively. At 3 hours post-dosing, the average levels of MCP-1 (CCL2) decreased to <1,000 pg/ml and <500 pg/ml in mice treated with the purified SIRPα-Fc-CD40L or TIGIT-Fc-LIGHT chimeric proteins, respectively (FIG. 11C). At 24 hours post-dosing, the average levels of MCP-1 (CCL2) dropped to background levels in mice treated with the purified SIRPα-Fc-CD40L or TIGIT-Fc-LIGHT chimeric proteins (FIG. 11C). At 48, 72, and 96-hours post-dosing, the average levels of MCP-1 (CCL2) remained at background levels in mice treated with the purified SIRPα-Fc-CD40L or TIGIT-Fc-LIGHT chimeric proteins (FIG. 11C). In contrast, as shown in FIG. 11C, at 1 hour post-dosing, the average levels of MCP-1 (CCL2) reached >5,000 pg/ml and >3,000 pg/ml in mice treated with the mmRNA encoding SIRPα-Fc-CD40L or TIGIT-Fc-LIGHT chimeric proteins, respectively. At 3 hours post-dosing, the average levels of MCP-1 (CCL2) remained >5,000 pg/ml and >3,000 pg/ml in mice treated with the mmRNA encoding SIRPα-Fc-CD40L or TIGIT-Fc-LIGHT chimeric proteins, respectively (FIG. 11C). Interestingly, the observed sustained average levels of MCP-1 (CCL2) contrasts with the precipitous decrease in the average levels of MCP-1 (CCL2) at 3 hours post dosing in mice treated with the purified SIRPα-Fc-CD40L or TIGIT-Fc-LIGHT chimeric proteins (FIG. 11C). Surprisingly at 24 hours, in contrast to the mice treated with the purified SIRPα-Fc-CD40L or TIGIT-Fc-LIGHT chimeric proteins, the average levels of MCP-1 (CCL2) remained >1,000 pg/ml and >750 pg/ml in mice treated with the mmRNA encoding SIRPα-Fc-CD40L or TIGIT-Fc-LIGHT chimeric proteins, respectively (FIG. 11C).

Collectively, these results demonstrate, inter alia, that mmRNA-encoded SIRPα-Fc-CD40L or TIGIT-Fc-LIGHT chimeric proteins generated an on-target serum cytokine response that is comparable to the fusion protein constructs. These results further demonstrate, inter alia, that the cytokine production via the mmRNA is more sustained compared to fusion proteins.

### Example 11: Immune Activation Induced by mmRNA Encoding the SIRPα-Fc-CD40L or TIGIT-Fc-LIGHT Chimeric Proteins

The in vivo changes in activated lymphocytes induced by LNP comprising 12.5 μg of modified mRNA (mmRNA) encoding the SIRPα-Fc-CD40L or TIGIT-Fc-LIGHT chimeric proteins were analyzed. Briefly, 1×10⁶ tumor cells were injected subcutaneously into the lower flanks of mice. When tumors became were palpable, the mice were randomly distributed in three treatment groups and treated as follows:
Group 1: empty LNP lacking mmRNA,
Group 2: 200 μg of the purified recombinant mouse SIRPα-Fc-CD40L chimeric protein, and
Group 3: LNP comprising 12.5 μg mmRNA encoding the mouse SIRPα-Fc-CD40L chimeric protein,

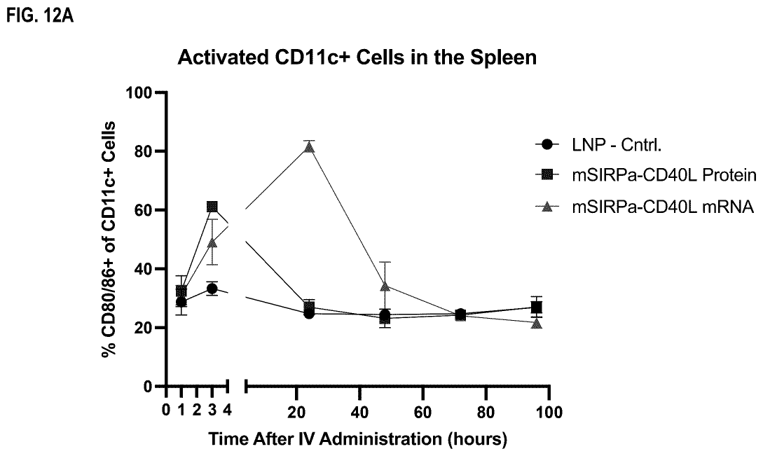

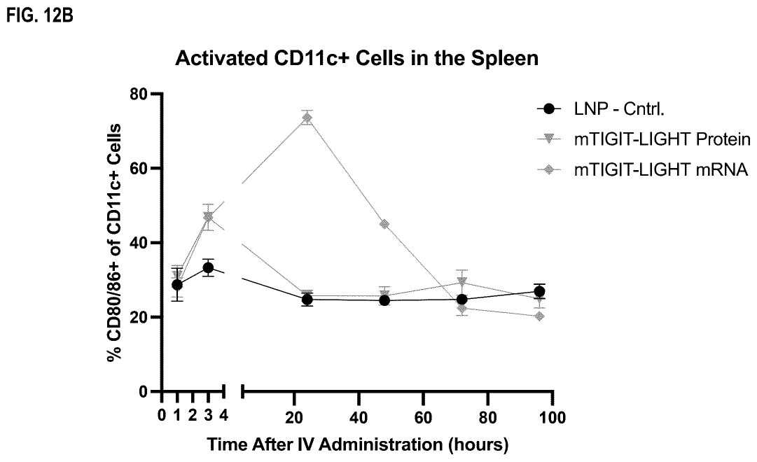

Mice were sacrificed at 1 hr, 3 hr, 24 hr, 48 hr, 72 hr and 96 hr post-treatment, and their spleens were recovered. The spleen samples were dissociated and CD11c+ cells were enriched from the dissociated tissue samples. The CD11c+-enriched cell population was stained with anti-CD11c and anti-CD80/CD86 antibodies and subjected to flow cytometry. The percentage of activated CD80/86+ cells (out of CD11c+) in CD11c+-enriched dissociated spleen tissue samples were determined. The mice treated with empty LNP showed a background level of activated (CD80/86+) cells (FIG. 12A). As shown in FIG. 12A, at 3 hours-post treatment, the mice treated with the purified SIRPα-Fc-CD40L chimeric protein showed about 2-fold increase in the levels of activated (CD80/86+) cells. The levels of activated (CD80/86+) cells induced by the purified SIRPα-Fc-CD40L chimeric protein decreased to background levels by 24 hours post-treatment and remained there at 48 hr, 72 hr and 96 hr post-treatment (FIG. 12A). As shown in FIG. 12A, the mice treated with the mmRNA encoding SIRPα-Fc-CD40L chimeric protein also showed an increase in the levels of activated (CD80/86+) cells at 3 hours-post treatment similar to mice treated with the purified SIRPα-Fc-CD40L chimeric protein. Surprisingly, the mice treated with the mmRNA encoding SIRPα-Fc-CD40L chimeric protein exhibited a further increase in the levels of activated (CD80/86+) cells at 24 hours-post treatment, in contrast to mice treated with the purified SIRPα-Fc-CD40L chimeric protein, which showed background levels of activated (CD80/86+) cells at 24 hours-post treatment (FIG. 12A). Interestingly, at 48 hours-post treatment, the mice treated with the mmRNA encoding SIRPα-Fc-CD40L chimeric protein exhibited the above-background levels of activated (CD80/86+) cells (FIG. 12A). The levels of activated (CD80/86+) cells in mice treated with mmRNA encoding the SIRPα-Fc-CD40L chimeric protein decreased to background levels by 72 hours post-treatment and remained there at 96 hr post-treatment (FIG. 12A).

The in vivo changes in activated lymphocytes induced by LNP comprising 12.5 μg of modified mRNA (mmRNA) encoding the TIGIT-Fc-LIGHT or TIGIT-Fc-LIGHT chimeric proteins were analyzed. Briefly, 1×10⁶ tumor cells were injected subcutaneously into the lower flanks of mice. When tumors became were palpable, the mice were randomly distributed in three treatment groups and treated as follows:
Group 1: empty LNP lacking mmRNA,
Group 2: 200 μg of the purified recombinant mouse TIGIT-Fc-LIGHT chimeric protein, and
Group 3: LNP comprising 12.5 μg mmRNA encoding the mouse TIGIT-Fc-LIGHT chimeric protein,

Mice were sacrificed at 1 hr, 3 hr, 24 hr, 48 hr, 72 hr and 96 hr post-treatment, and their spleens were recovered. The spleen samples were dissociated and CD11c+ cells were enriched from the dissociated tissue samples. The CD11c+-enriched cell population was stained with anti-CD11c and anti-CD80/CD86 antibodies and subjected to flow cytometry. The percentage of activated CD80/86+ cells (out of CD11c+) in CD11c+-enriched dissociated spleen tissue samples were determined. The mice treated with empty LNP showed a background level of activated (CD80/86+) cells (FIG. 12B). As shown in FIG. 12B, at 3 hours-post treatment, the mice treated with the purified TIGIT-Fc-LIGHT chimeric protein showed about 1.5-fold increase in the levels of activated (CD80/86+) cells. The levels of activated (CD80/86+) cells induced by the purified TIGIT-Fc-LIGHT chimeric protein decreased to background levels by 24 hours post-treatment and remained there at 48 hr, 72 hr and 96 hr post-treatment (FIG. 12B). As shown in FIG. 12B, the mice treated with the mmRNA encoding TIGIT-Fc-LIGHT chimeric protein also showed an increase in the levels of activated (CD80/86+) cells at 3 hours-post treatment similar to mice treated with the purified TIGIT-Fc-LIGHT chimeric protein. Surprisingly, the mice treated with the mmRNA encoding TIGIT-Fc-LIGHT chimeric protein exhibited a further increase in the levels of activated (CD80/86+) cells at 24 hours-post treatment, in contrast to mice treated with the purified TIGIT-Fc-LIGHT chimeric protein, which showed background levels of activated (CD80/86+) cells at 24 hours-post treatment (FIG. 12B). Interestingly, at 48 hours-post treatment, the mice treated with the mmRNA encoding TIGIT-Fc-LIGHT chimeric protein exhibited the above-background levels of activated (CD80/86+) cells (FIG. 12B). The levels of activated (CD80/86+) cells in mice treated with mmRNA encoding the TIGIT-Fc-LIGHT chimeric protein decreased to background levels by 72 hours post-treatment and remained there at 96 hr post-treatment (FIG. 12B).

These results indicate, inter alia, a sustained activation immune cells (e.g., CD80/CD86+ cells in the CD11c+ splenic dendritic cells) following the administration of mmRNA encoding the chimeric proteins of the present disclosure. Accordingly, mmRNA encoding the chimeric proteins of the present disclosure are likely to cause sustained immune activation, leading to superior anti-cancer efficacy.

### Example 12: In Vivo Efficacy of mmRNA Encoding the SIRPα-Fc-CD40L or TIGIT-Fc-LIGHT Chimeric Proteins

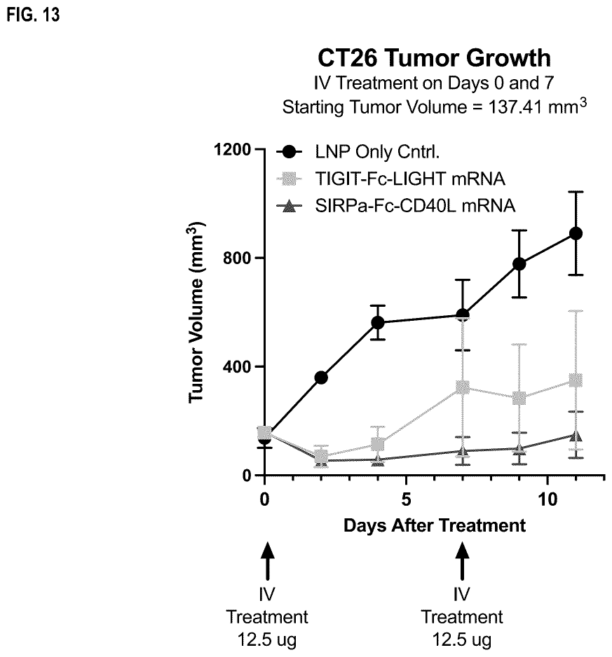

The anti-tumor efficacy of modified mRNA (mmRNA) encoding the SIRPα-Fc-CD40L or TIGIT-Fc-LIGHT chimeric proteins was studied in comparison with vehicle-alone treated mice. Briefly, 1×10⁶ tumor cells were injected subcutaneously into the lower flanks of mice. When tumors became were palpable (average starting tumor volume reached 137.41 mm³ on day 0), the mice were randomly distributed in three treatment groups and treated intravenously on days 0 and 4 (indicated by arrows in FIG. 13) as follows:
Group 1: empty LNP lacking mmRNA,
Group 2: LNP comprising 12.5 μg mmRNA encoding the mouse SIRPα-Fc-CD40L chimeric protein, and
Group 3: LNP comprising 12.5 μg mmRNA encoding the mouse TIGIT-Fc-LIGHT chimeric protein.

Tumors were measured with electronic calipers on days 2, 4, 7, 9 and 11 and plotted using the GraphPad Prism software. As shown in FIG. 13, the mice treated with empty LNP (circles in FIG. 13) showed rapid growth of tumors. In contrast, the mice treated with mmRNA encoding the mouse SIRPα-Fc-CD40L chimeric protein (triangles in FIG. 13) or mmRNA encoding the mouse TIGIT-Fc-LIGHT chimeric protein (squares in FIG. 13) showed control of tumor growth.

These results indicate, inter alia, that mmRNA encoding the chimeric proteins of the present disclosure may be beneficial in the treatment of cancer, and thus, may be useful in the methods disclosed herein.

### Example 13: In Vitro Expression Delivery of DNA Minicircle Encoding the SIRPα-Fc-CD40L Chimeric Protein

To assess feasibility of nucleic acid-based delivery of the SIRPα-Fc-CD40L chimeric protein, minicircles were used. The minicircle comprises a small circular DNA molecule that utilizes a transduced cell's transcription and translation machinery to produce an encoded gene product. The minicircle was devoid of all prokaryotic components, leaving only mammalian elements. They are produced as small circular DNA fragments that are generated by excising any bacterial components of from a parental plasmid. They are non-viral and remain episomal. Their small size facilitates more efficient transfection than what's possible with standard-sized plasmids. Minicircles do not replicate with the host cell, expression lasts for 14 days or longer in dividing cells, and can continue for months in non-dividing cells.

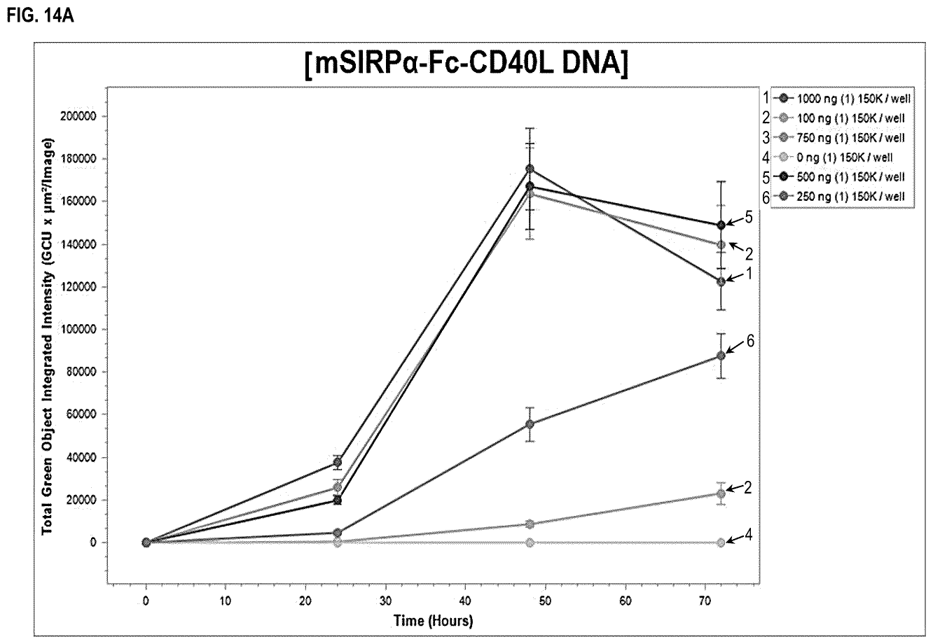

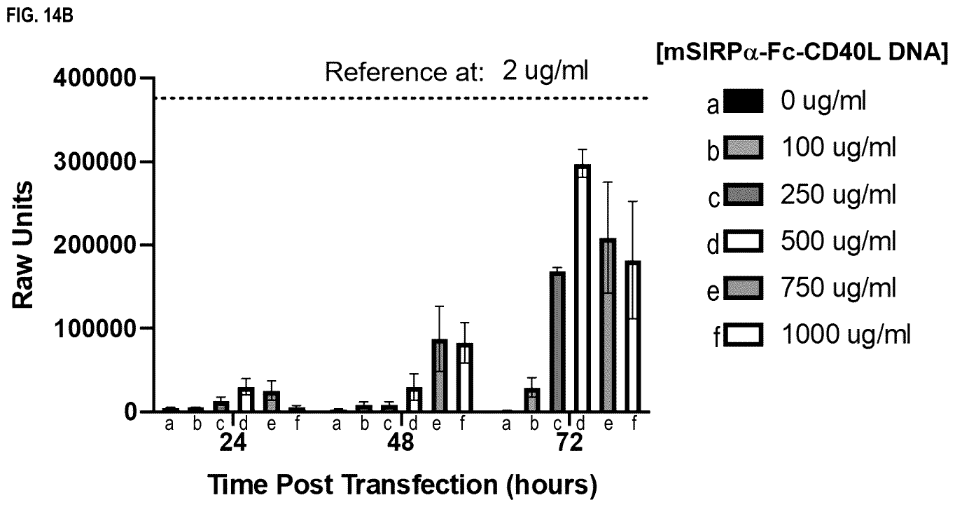

HEK293T cells were transfected with 100, 250, 500 750 or 1000 ng a DNA minicircle co-expressing GFP and mouse SIRPα-Fc-CD40L chimeric protein. Vehicle-only transfection was used as a control. GFP expression was assessed by measuring fluorescence using the INCUCYTE system, which allows live-cell imaging of a fluorescence signal. GFP expression was measured by time-lapse microscopy for 72 hours. As shown in FIG. 14A, the cells showed a dose-dependent increase in GFP expression that peaked between 48 and 72 hours.

To analyze the expression of the SIRPα-Fc-CD40L chimeric protein, a dual antibody Meso Scale Discovery (MSD) ELISA assay was carried out. Briefly, an anti-SIRPα antibody was coated on plates and increasing amounts of the cell culture supernatants recovered after 24, 48 or 72 hours were added to the plate for capture by the plate-bound anti-SIRPα antibody. Binding of the SIRPα-Fc-CD40L chimeric protein to the anti-SIRPα antibody was detected using an electrochemiluminescence (ECL) readout using a SULFO-tagged anti-CD40L antibody. This assay detects the intact SIRPα-Fc-CD40L chimeric protein because detection requires simultaneous binding to anti-SIRPα and anti-CD40L antibodies. As shown in FIG. 14B, a dose-dependent increase in the expression of the SIRPα-Fc-CD40L chimeric protein was observed at 24, 48 or 72 hours. Further, the expression of the SIRPα-Fc-CD40L chimeric protein increased from 24 to 48 and to 72 hours.

These results demonstrate, inter alia, that the DNA minicircle co-expressing GFP and mouse SIRPα-Fc-CD40L chimeric protein could drive the expression of the SIRPα-Fc-CD40L chimeric protein in vitro.

### Example 14: In Vivo Nucleic Acid-Based Delivery of the SIRPα-Fc-CD40L Chimeric Protein

Mice were administered vehicle only control, the purified SIRPα-Fc-CD40L chimeric protein (intraperitoneal (IP)) or 15 μg DNA minicircle expressing the SIRPα-Fc-CD40L chimeric protein (intravenous (IV)). Four days after treatment blood serum, bone marrow, and livers were isolated from treated animals. Tissue was dissociated and levels of SIRPα-Fc-CD40L were detected using a fusion protein-specific ELISA.

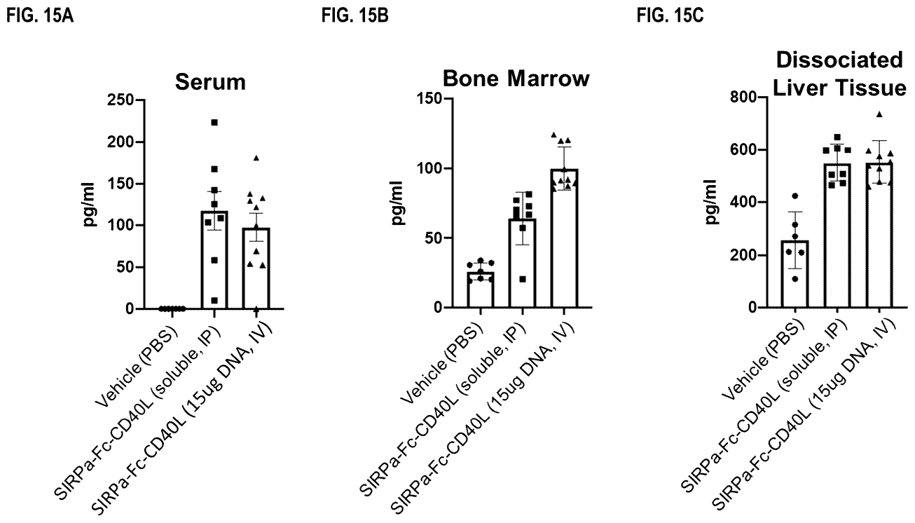

Briefly, an anti-SIRPα antibody was coated on plates and increasing amounts of the serum, dissociated samples of bone marrow or liver were added to the plate for capture by the plate-bound anti-SIRPα antibody. Binding of the SIRPα-Fc-CD40L chimeric protein to the anti-SIRPα antibody was detected using an electrochemiluminescence (ECL) readout using a SULFO-tagged anti-CD40L antibody. This assay detects the intact SIRPα-Fc-CD40L chimeric protein because detection requires simultaneous binding to anti-SIRPα and anti-CD40L antibodies. As shown in FIG. 15A, the SIRPα-Fc-CD40L chimeric protein could be detected in serum of the mice that were administered the purified SIRPα-Fc-CD40L chimeric protein or the DNA minicircle expressing the SIRPα-Fc-CD40L chimeric protein. As expected, the serum of mice injected with vehicle only showed only a background signal (FIG. 15A). As shown in FIG. 15B, the SIRPα-Fc-CD40L chimeric protein could be detected in the dissociated bone marrow samples of the mice that were administered the purified SIRPα-Fc-CD40L chimeric protein or the DNA minicircle expressing the SIRPα-Fc-CD40L chimeric protein. As expected, the dissociated bone marrow samples of mice injected with vehicle only showed only a background signal (FIG. 15B). As shown in FIG. 15C, the SIRPα-Fc-CD40L chimeric protein could be detected in the dissociated liver samples of the mice that were administered the purified SIRPα-Fc-CD40L chimeric protein or the DNA minicircle expressing the SIRPα-Fc-CD40L chimeric protein. As expected, the dissociated liver samples of mice injected with vehicle only showed only a background signal (FIG. 15B).

These results demonstrate, inter alia, that delivery of a nucleic acid encoding the SIRPα-Fc-CD40L chimeric protein leads to the expression of the SIRPα-Fc-CD40L chimeric protein in vivo.

### Example 15: In Vivo Activity of the Nucleic Acid-Based Delivered SIRPα-Fc-CD40L Chimeric Protein

We have previously shown in mouse, cynomolgus macaque, and humans that IP (mouse) and IV (NHP and human) delivery of the SIRPα-Fc-CD40L chimeric protein leads to a margination of CD40+ B cells out of the peripheral blood. See Lakhani et al., Phase 1 dose escalation study of the agonist redirected checkpoint, SL-172154 (SIRPα-Fc-CD40L) in subjects with platinum-resistant ovarian cancer (NCT04406623), SITC meeting, 2021.

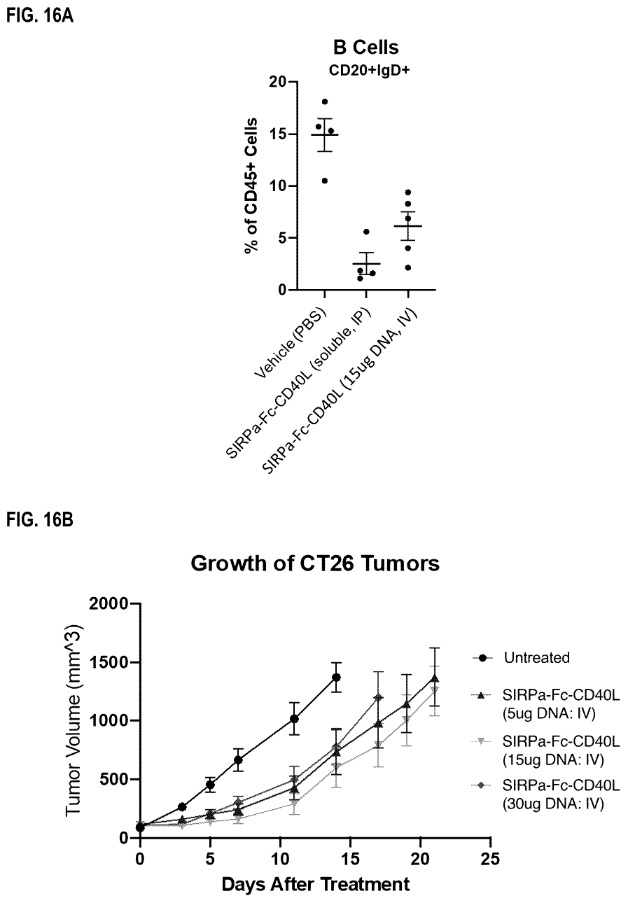

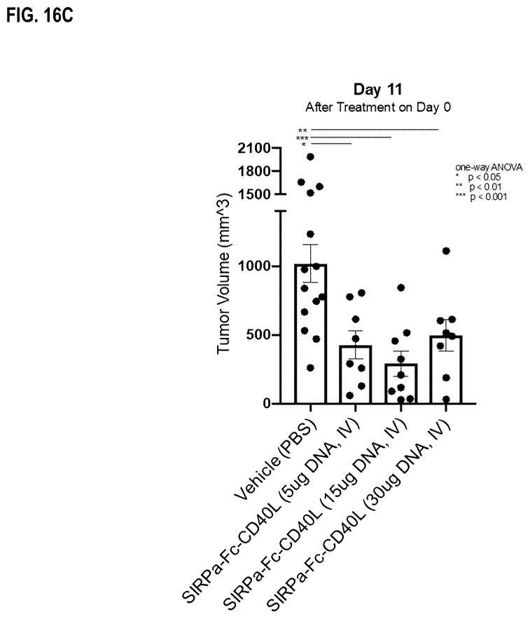

To understand whether delivery of a nucleic acid encoding the SIRPα-Fc-CD40L chimeric protein leads to margination, CD20+IgD+ B cells were measured out of the total CD45+ cells by flow cytometry. The marginated B cells are most likely CD40+. As shown in FIG. 16A, compared to vehicle-treated mice, administration of either the purified SIRPα-Fc-CD40L chimeric protein or the DNA minicircle expressing the SIRPα-Fc-CD40L chimeric protein led to a margination of B cells. This is consistent with previous observations. See, e.g., Lakhani et al., Phase 1 dose escalation study of the agonist redirected checkpoint, SL-172154 (SIRPα-Fc-CD40L) in subjects with platinum-resistant ovarian cancer (NCT04406623), SITC meeting, 2021.

These results demonstrate, inter alia, that in vivo expression and biological activity of the SIRPα-Fc-CD40L chimeric protein may be achieved by delivery of a nucleic acid encoding the SIRPα-Fc-CD40L chimeric protein.

Mice were inoculated with murine CT26 colorectal carcinoma cancer cells on the hind flank, and when tumors became palpable tumors, and when tumors were palpable, the mice were given a single intravenous (IV) injection of vehicle only, or 5 μg, 15 μg, or 30 μg of the DNA minicircle encoding the SIRPα-Fc-CD40L chimeric protein. Tumor volumes were measured over a 21-day time course. SIRPα show the tumor growth curves. As shown in FIG. 16B, the tumors grew aggressively in vehicle only-treated mice. In contrast, the mice treated with the DNA minicircle encoding the SIRPα-Fc-CD40L chimeric protein showed a reduction in tumor growth (FIG. 16B).

Tumor sizes on day 11 were plotted. As shown FIG. 16C, compared to the average tumor volumes in vehicle only-treated mice, there was a significant reduction in the average tumor volumes in mice treated with 5 μg (p<0.05), 15 μg (p<0.001), or 30 μg (p<0.001) of the DNA minicircle encoding the SIRPα-Fc-CD40L chimeric protein.

These results demonstrate, inter alia, that a nucleic acid-based delivery of the SIRPα-Fc-CD40L chimeric protein causes tumor shrinkage.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A method for treating a cancer, an autoimmune condition, or an inflammatory disorder subject comprising a step of administering to the subject a pharmaceutical composition comprising an isolated polynucleotide encoding a chimeric protein having a general structure of: 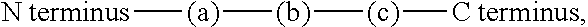
wherein:
(a) is a first domain comprising an extracellular domain of T cell immunoreceptor with Ig and ITIM domains (TIGIT), or a variant or a fragment thereof that is capable of binding a TIGIT ligand,
(c) is a second domain comprising an extracellular domain of glucocorticoid-induced TNFR-related protein ligand (GITRL), or a variant or a fragment thereof that is capable of binding a GITRL receptor, or LIGHT (homologous to lymphotoxin, exhibits inducible expression and competes with HSV glycoprotein D for binding to herpesvirus entry mediator, a receptor expressed on T lymphocytes), or a variant or a fragment thereof that is capable of binding a LIGHT receptor,
(b) is a linker adjoining the first and second domains, wherein the linker comprises at least one cysteine residue capable of forming a disulfide bond and/or comprises a hinge-CH2-CH3 Fc domain.

2. The method of claim 1, wherein the TIGIT ligand is selected from CD155/PVR, Nectin-2, Nectin-3 and Nectin-4.

3. The method of claim 1 or claim 2, wherein the GITRL receptor is glucocorticoid-induced TNFR-related protein (GITR).

4. The method of claim 1 or claim 2, wherein the LIGHT receptor is TNFRSF3/LTBR.

5. A method for treating a cancer, an autoimmune condition, or an inflammatory disorder subject comprising a step of administering to the subject a pharmaceutical composition comprising an isolated polynucleotide encoding a chimeric protein having a general structure of: 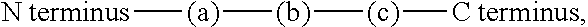
wherein:
(a) is a first domain comprising an extracellular domain of programmed death-1 (PD-1), or a variant or a fragment thereof that capable of binding a PD-1 ligand,
(c) is a second domain comprising an extracellular domain of OX40 ligand (OX40L), or a variant or a fragment thereof that capable of binding an OX40L receptor, or 4-1BB Ligand (4-1BBL), or a variant or a fragment thereof that capable of binding a 4-1BBL receptor,
(b) is a linker adjoining the first and second domains, wherein the linker comprises at least one cysteine residue capable of forming a disulfide bond and/or comprises a hinge-CH2-CH3 Fc domain.

6. The method of claim 5, wherein the PD-1 ligand is PD-L1 or PD-L2.

7. The method of claim 5 or claim 6, wherein the OX40L receptor is OX40.

8. The method of claim 5 or claim 6, wherein the 4-1BBL receptor is 4-1BB.

9. A method for treating a cancer, an autoimmune condition, or an inflammatory disorder subject comprising a step of administering to the subject a pharmaceutical composition comprising an isolated polynucleotide encoding a chimeric protein having a general structure of: 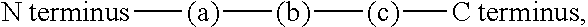
wherein:
(a) is a first domain comprising an extracellular domain of T-cell immunoglobulin mucin receptor 3 (TIM3), or a variant or a fragment thereof that capable of binding a TIM3 ligand,
(c) is a second domain comprising an extracellular domain of CD40 ligand (CD40L), or a variant or a fragment thereof that capable of binding a CD40L receptor,
(b) is a linker adjoining the first and second domains, wherein the linker comprises at least one cysteine residue capable of forming a disulfide bond and/or comprises a hinge-CH2-CH3 Fc domain.

10. The method of claim 9, wherein the TIM3 ligand is selected from galectin 9, phosphatidylserine (PtdSer), CEACAM1 and high mobility group protein B1 (HMGB1).

11. The method of claim 9 or claim 10, wherein the CD40L receptor is CD40.

12. A method for treating a cancer, an autoimmune condition, or an inflammatory disorder subject comprising a step of administering to the subject a pharmaceutical composition comprising an isolated polynucleotide encoding a chimeric protein having a general structure of: 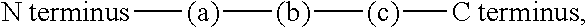
wherein:
(a) is a first domain comprising an extracellular domain of V-set and immunoglobulin domain-containing protein 8 (VSIG8), or a variant or a fragment thereof that capable of binding a VSIG8 ligand,
(c) is a second domain comprising an extracellular domain of 4-1BB Ligand (4-1BBL), or a variant or a fragment thereof that capable of binding a 4-1BBL receptor,
(b) is a linker adjoining the first and second domains, wherein the linker comprises at least one cysteine residue capable of forming a disulfide bond and/or comprises a hinge-CH2-CH3 Fc domain.

13. The method of claim 12, wherein the 4-1BBL receptor is 4-1BB.

14. A method for treating a cancer, an autoimmune condition, or an inflammatory disorder subject comprising a step of administering to the subject a pharmaceutical composition comprising an isolated polynucleotide encoding a chimeric protein having a general structure of: 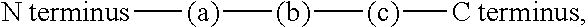
wherein:
(a) is a first domain comprising an extracellular domain of signal regulatory protein a (SIRPα), or a variant or a fragment thereof that is capable of binding a SIRPα ligand,
(c) is a second domain comprising an extracellular domain of CD40 ligand (CD40L), or a variant or a fragment thereof that is capable of binding a CD40L receptor,
(b) is a linker adjoining the first and second domains, wherein the linker comprises at least one cysteine residue capable of forming a disulfide bond and/or comprises a hinge-CH2-CH3 Fc domain.

15. The method of claim 14, wherein the SIRPα ligand is CD47.

16. The method of claim 14, wherein the CD40L receptor is CD40.

17. The method of any one of claims 1 to 16, wherein the hinge-CH2-CH3 Fc domain is derived from IgG1, optionally human IgG1.

18. The method of any one of claims 1 to 17, wherein the linker comprises the hinge-CH2-CH3 Fc domain is derived from IgG4, optionally human IgG4.

19. The method of claim 17 or claim 18, wherein the hinge-CH2-CH3 Fc domain comprises an amino acid sequence that is at least about 95% identical to the amino acid sequence of SEQ ID NO: 1, SEQ ID NO: 2, SEQ ID NO: 3, SEQ ID NO: 4, or SEQ ID NO: 5.

20. The method of any one of claims 1 to 19, wherein the linker further comprises wherein the linker further comprises the linker comprises one or more joining linkers, such joining linkers independently selected from SEQ ID NOs: 6 to 52, optionally wherein the linker comprises two or more joining linkers each joining linker independently selected from SEQ ID NOs: 6 to 52; wherein one joining linker is N terminal to the hinge-CH2-CH3-Fc domain and another joining linker is C terminal to the hinge-CH2-CH3-Fc domain.

21. The method of any one of claims 1 to 20, wherein the polynucleotide is or comprises an mRNA or a modified mRNA (mmRNA).

22. The method of claim 21, wherein the polynucleotide is or comprises an mmRNA.

23. The method of claim 22, wherein the mmRNA further comprises a 3′ untranslated region (UTR).

24. The method of claim 23, wherein the 3′ UTR comprises at least one microRNA-122 (miR-122) binding site.

25. The method of claim 24, wherein the miR-122 binding site is a miR-122-3p binding site or a miR-122-5-binding site.

26. The method of claim 24 or claim 25, wherein the mmRNA further comprises a spacer sequence between the open reading frame and the miRNA binding site.

27. The method of claim 26, wherein the spacer sequence comprises at least about 10 nucleotides, at least about 20 nucleotides, at least about 30 nucleotides, at least about 40 nucleotides, at least about 50 nucleotides, at least about 60 nucleotides, at least about 70 nucleotides, at least about 80 nucleotides, at least about 90 nucleotides, or at least about 100 nucleotides.

28. The method of any one of claims 21-27, wherein the mmRNA further comprises a 5′ UTR.

29. The method of claim 28, wherein the 5′ UTR harbors a Kozak sequence and/or forms a secondary structure that stimulate elongation factor binding.

30. The method of any one of claims 21-29, wherein the mmRNA further comprises a 5′ terminal cap.

31. The method of claim 30, wherein the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

32. The method of any one of claims 21-31, wherein the mmRNA comprises one or more modifications.

33. The method of claim 32, wherein the modifications are selected from:
pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and a combination of any two or more thereof; and/or
wherein the modifications are selected from pseudouridine (Ψ), N1-methylpseudouridine (m1Ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1L), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methylinosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and a combination of any two or more thereof.

34. The method of any one of claim 31 or 32, wherein modification is selected from pseudouridine, N1-methylpseudouridine, 5-methylcytosine, 5-methoxyuridine, and a combination thereof.

35. The method of any one of claims 31 to 34, wherein the mmRNA comprises at least one N1-methylpseudouridine.

36. The method of any one of claims 31 to 35, wherein the mmRNA is fully modified with chemically-modified uridines.

37. The method of claim 36, wherein the mmRNA is a fully modified with N1-methylpseudouridine.

38. The method of any one of claims 31 to 37, wherein the modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine or a combination of any two or more thereof.

39. The method of any one of claims 31 to 38, wherein the modifications are selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

40. The method of any one of claims 31 to 39, wherein the modifications are selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

41. The method of any one of claims 31 to 40, wherein the modifications are selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

42. The method of any one of claims 31 to 41, wherein the modifications are present on the major groove face.

43. The method of claim 42, wherein a hydrogen on C-5 of uracil is replaced with a methyl group or a halo group.

44. The method of any one of claims 31 to 43, wherein the mmRNA further comprises one or more modifications selected from 5′-O-(1-Thiophosphate)-Adenosine, 5′-O-(1-Thiophosphate)-Cytidine, 5′-O-(1-Thiophosphate)-Guanosine, 5′-O-(1-Thiophosphate)-Uridine and 5′-O-(1-Thiophosphate)-Pseudouridine.

45. The method of any one of claims 1 to 44, wherein the pharmaceutical composition further comprises a lipidoid, a liposome, a lipoplex, a lipid nanoparticle, a polymeric nanoparticle, a peptide, a protein, a cell, a nanoparticle mimic, a nanotube, or a conjugate.

46. The method of claim 45, wherein the pharmaceutical composition is formulated as a lipid nanoparticle (LNP), a lipoplex, or a liposome.

47. The method of claim 46, wherein the pharmaceutical composition is formulated as a lipid nanoparticle (LNP).

48. The method of claim 47, wherein the LNP comprises a molar ratio of about 20-60% ionizable amino lipid, about 5-25% phospholipid, about 25-55% structural lipid, and about 0.5-1.5% PEG lipid.

49. The method of claim 47 or claim 48, wherein the LNP comprises a molar ratio of about 50% ionizable amino lipid, about 8-12% phospholipid, about 37-40% structural lipid, and about 1-2% PEG lipid.

50. The method of any one of claims 47 to 49, wherein the lipid nanoparticles comprise lipids selected from an ionizable lipid (e.g., an ionizable cationic lipid selected from DLin-DMA, DLin-K-DMA, DLin-KC2-DMA, DLin-MC3-DMA, 98N12-5, and C12-200); a structural lipid (e.g., distearoylphosphatidylcholine (DSPC)); cholesterol, and a polyethyleneglycol (PEG)-lipid (e.g., a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); dioleoylphosphatidylethanolamine (DOPE).

51. The method of any one of claims 47 to 50, wherein the lipid nanoparticles comprise (a) a cationic lipid comprising from 50 mol % to 85 mol % of the total lipid present in the particle; (b) a non-cationic lipid comprising from 13 mol % to 49.5 mol % of the total lipid present in the particle; and (c) a conjugated lipid that inhibits aggregation of particles comprising from 0.5 mol % to 2 mol % of the total lipid present in the particle.

52. The method of any one of claims 47 to 51, wherein the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

53. The method of any one of claims 1 to 52, wherein the pharmaceutical composition is formulated for parenteral administration.

54. A pharmaceutical composition comprising an isolated modified mRNA (mmRNA) encoding a heterologous chimeric protein having an amino acid sequence that has at least about 95% sequence identity with an amino acid sequence selected from SEQ ID NOs: 80, 81, and 90-93.

55. The pharmaceutical composition of claim 54, wherein the mmRNA comprises a 3′ untranslated region (UTR).

56. The pharmaceutical composition of claim 55, wherein the 3′ UTR comprises at least one microRNA-122 (miR-122) binding site, optionally wherein the miR-122 binding site is a miR-122-3p binding site or a miR-122-5-binding site.

57. The pharmaceutical composition of any one of claims 54 to 56, wherein the mmRNA further comprises a spacer sequence between the open reading frame and the miRNA binding site, optionally wherein the spacer sequence comprises at least about 10 nucleotides, at least about 20 nucleotides, at least about 30 nucleotides, at least about 40 nucleotides, at least about 50 nucleotides, at least about 60 nucleotides, at least about 70 nucleotides, at least about 80 nucleotides, at least about 90 nucleotides, or at least about 100 nucleotides.

58. The pharmaceutical composition of any one of claims 54-57, wherein the mmRNA further comprises a 5′ UTR, optionally wherein the 5′ UTR harbors a Kozak sequence and/or forms a secondary structure that stimulate elongation factor binding.

59. The pharmaceutical composition of any one of claims 54-58, wherein the mmRNA further comprises a 5′ terminal cap.

60. The pharmaceutical composition of claim 59, wherein the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

61. The pharmaceutical composition of any one of claims 54-60, wherein the mmRNA comprises one or more modifications.

62. The pharmaceutical composition of claim 61, wherein the modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and a combination of any two or more thereof.

63. The pharmaceutical composition of claim 61 or claim 62, wherein the modifications are selected from pseudouridine (Ψ), N1-methylpseudouridine (m1Ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1Ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methylinosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and a combination of any two or more thereof.

64. The pharmaceutical composition of any one of claims 61 to 63, wherein modification is selected from pseudouridine, N1-methylpseudouridine, 5-methylcytosine, 5-methoxyuridine, and a combination thereof.

65. The pharmaceutical composition of any one of claims 61 to 64, wherein the mmRNA comprises at least one N1-methylpseudouridine.

66. The pharmaceutical composition of any one of claims 61 to 65, wherein the mmRNA is fully modified with chemically-modified uridines.

67. The pharmaceutical composition of claim 66, wherein the mmRNA is a fully modified with N1-methylpseudouridine.

68. The pharmaceutical composition of any one of claims 61 to 67, wherein the modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine or a combination of any two or more thereof.

69. The pharmaceutical composition of any one of claims 61 to 68, wherein the modifications are selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

70. The pharmaceutical composition of any one of claims 61 to 69, wherein the modifications are selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

71. The pharmaceutical composition of any one of claims 61 to 70, wherein the modifications are selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

72. The pharmaceutical composition of any one of claims 61 to 71, wherein the modifications are present on the major groove face.

73. The pharmaceutical composition of claim 72, wherein a hydrogen on C-5 of uracil is replaced with a methyl group or a halo group.

74. The pharmaceutical composition of any one of claims 71 to 73, wherein the mmRNA further comprises one or more modifications selected from 5′-O-(1-Thiophosphate)-Adenosine, 5′-O-(1-Thiophosphate)-Cytidine, 5′-O-(1-Thiophosphate)-Guanosine, 5′-O-(1-Thiophosphate)-Uridine and 5′-O-(1-Thiophosphate)-Pseudouridine.

75. The pharmaceutical composition of any one of claims 54 to 74, wherein the pharmaceutical composition further comprises a lipidoid, a liposome, a lipoplex, a lipid nanoparticle, a polymeric nanoparticle, a peptide, a protein, a cell, a nanoparticle mimic, a nanotube, or a conjugate.

76. The pharmaceutical composition of claim 75, wherein the pharmaceutical composition is formulated as a lipid nanoparticle (LNP), a lipoplex, or a liposome.

77. The pharmaceutical composition of claim 76, wherein the pharmaceutical composition is formulated as a lipid nanoparticle (LNP).

78. The pharmaceutical composition of claim 77, wherein the LNP comprises a molar ratio of about 20-60% ionizable amino lipid, about 5-25% phospholipid, about 25-55% structural lipid, and about 0.5-1.5% PEG lipid.

79. The pharmaceutical composition of claim 77 or claim 78, wherein the LNP comprises a molar ratio of about 50% ionizable amino lipid, about 8-12% phospholipid, about 37-40% structural lipid, and about 1-2% PEG lipid.

80. The pharmaceutical composition of any one of claims 77 to 79, wherein the lipid nanoparticles comprise lipids selected from an ionizable lipid (e.g., an ionizable cationic lipid selected from DLin-DMA, DLin-K-DMA, DLin-KC2-DMA, DLin-MC3-DMA, 98N12-5, and C12-200); a structural lipid (e.g., distearoylphosphatidylcholine (DSPC)); cholesterol, and a polyethyleneglycol (PEG)-lipid (e.g., a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); dioleoylphosphatidylethanolamine (DOPE).

81. The pharmaceutical composition of any one of claims 77 to 80, wherein the lipid nanoparticles comprise (a) a cationic lipid comprising from 50 mol % to 85 mol % of the total lipid present in the particle; (b) a non-cationic lipid comprising from 13 mol % to 49.5 mol % of the total lipid present in the particle; and (c) a conjugated lipid that inhibits aggregation of particles comprising from 0.5 mol % to 2 mol % of the total lipid present in the particle.

82. The pharmaceutical composition of any one of claims 77 to 81, wherein the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

83. The pharmaceutical composition of any one of claims 77 to 82, wherein the heterologous chimeric protein has an amino acid sequence that has at least about 95%, or at least about 96%, or at least about 97%, or at least about 98%, or at least about 99% sequence identity with an amino acid sequence selected from SEQ ID NOs: 80, 81, and 90-93.

84. A pharmaceutical composition comprising an isolated modified mRNA (mmRNA) encoding a heterologous chimeric protein having an amino acid sequence that has at least about 95%, or at least about 96%, or at least about 97%, or at least about 98%, or at least about 99% sequence identity with an amino acid sequence selected from SEQ ID NOs: 80, 81, and 90-93.

85. The pharmaceutical composition of any one of claims 54 to 81, wherein the pharmaceutical composition is formulated for parenteral administration.

86. A method for treating a cancer, an autoimmune condition, or an inflammatory disorder subject comprising a step of administering to the subject the pharmaceutical composition of any one of claims 54 to 82.
